(19) United States
(12) Patent Application Publication
(10) Pub. No.: US 2005/0255538 A1
(43) Pub. Date: Nov. 17, 2005

(54) Methods for screening for AcrAB efflux pump inhibitors

(76) Inventors: Stephen Earl Buxser, Kalamazoo, MI (US); Keith Poole, Kingston (CA); Douglas E. Decker, Kalamazoo, MI (US); Xianzhi Li, Ottawa (CA)

Correspondence Address: COZEN O'CONNOR, P.C., 1900 MARKET STREET, PHILADELPHIA, PA, 19103-3508, US

(21) Appl. No.: 10/392,234

(22) Filed: Mar. 17, 2003

Related U.S. Application Data
(60) Provisional application No. 60/364,935, filed on Mar. 15, 2002.

(51) Int. Cl. G01N 33/53; C12Q 1/02

(52) U.S. Cl. 435/32

(57) ABSTRACT

The invention relates to the field of antimicrobial agents, and to methods for the identification and characterization of potential antimicrobial agents and compounds which enhance the effect of antimicrobial agents. More specifically this invention relates to methods for screening agents for which the mode of action involves the acrAB family of efflux pumps.

### CROSS REFERENCE TO RELATED APPLICATIONS

This application claims priority to U.S. Provisional Ser. No. 60/364,935 filed Mar. 15, 2002, which is incorporated herein by reference.

### FIELD OF THE INVENTION

The invention relates to the field of antimicrobial agents, to methods for the identification and characterization of potential antimicrobial agents and compounds which enhance the effect of antimicrobial agents. More specifically, this invention relates to methods for screening agents for which the mode of action involves the acrAB family of efflux pumps.

### BACKGROUND OF THE INVENTION

Drug resistant strains of bacteria are an increasing threat to human health (Chopra et al., (1997) Antimicrob Agents Cemother, 41(3), 497-503; Travis (1994) Science 264(5157), 360-2). Resistance mechanisms include enzymatic modification of drugs, mutation at the drug target site, and the active efflux of drugs out of the cell by active transport (Levy (1992), Antimicrob Agents Chemother 36(4), 695-703; Williams (1996) Br J Biomed Sci 53(4), 290-3).

In gram-negative bacteria, the majority of multidrug transporters share a common three-component organization: a transporter located in the inner membrane (IM) functions with an outer membrane (OM) channel and a periplasmic accessory protein. In this arrangement, efflux complexes traverse both the inner and the outer membranes and thus facilitate direct passage of the substrate from the cytoplasm or the cytoplasmic membrane into the external medium. Direct efflux of drugs into the medium is advantageous for gram-negative bacteria because, in order to reenter the cell, the expelled drug molecules must cross the low permeability OM. Hence, drug efflux works synergistically with the low permeability of the OM. The synergy explains the observation that Gram-negative bacteria become hypersusceptible to various drugs either by the inactivation of efflux pumps or by the permeabilization of the OM.

In a recent review, Saier (Saier et al., (1998) Faseb J 12(3), 265-74) identified the known and putative transport proteins in Escherichia coli, Haemophilis influenza, Mycoplasma genitalium, Bacillus subtilis, Methanococcus jannechii, and Synechocystis PCC8603 for which the complete genome sequence is known. Drug efflux pumps were found in four families including the ATP binding cassette (ABC) superfamily, the major facilitator superfamily (MSF), the small multidrug resistance (SMR) family, and the resistance-nodulation-cell division (RND) family. In E. coli alone, 29 proven transport proteins were identified, 9 of which have been shown to pump drugs out of the cell. These results illustrate the importance of drug efflux as a major mechanism for drug resistance.

During the last few years it has become increasingly clear that multidrug efflux pumps, especially those containing the resistance nodulation division (RND) family transporters (Saier et al., (1994) Mol Microbiol 11(5), 8417) play a major role in producing both the intrinsic and the elevated levels of resistance to a very wide range of noxious compounds in gram-negative bacteria (Ma et al., (1994) J Bacteriol 175(19), 489-493, Nikaido (1994) Science 264(5157), 382-8, Nikaido, H. (1996) J Bacteriol 178(20), 5853-9).

Within the RND family of transporters, the acrAB system of Escherichia coli (Ma et al., 1993; Ma et al., (1995) Mol Microbiol 16(1) 45-55; Nakamura (1966) J Bacteriol 92(5), 1447-52.) and the Pseudomonas aeruginosa acrAB homologue (mexAB-oprM) (Li et al., (1995) Antimicrob Agents Chemother 39(9), 1948-53, Poole et al., (1993) J Bacteriol 175(22), 7363-72) are particularly well studied. The acrAB operon of E. coli transcribes a polycistronic message which encodes AcrA and AcrB. AcrB, the integral, inner-membrane spanning protein, is a 1048-residue protein with 12 putative transmembrane α-helices. The protein belongs to the RND (resistance-nodulation-cell division) transporter family (Saier 1994), which appears to catalyze efflux at the expense of proton motive force. The AcrAB system transports drug molecules directly into the medium, bypassing the periplasm. A periplasmic protein, AcrA, is required for the function of AcrB in intact cells (Ma 1995). AcrA has an unusual, elongated form, with a predicted length of 17 nm, in agreement with the idea that AcrA links or fuses inner and outer membranes. The third component of the system, an outer membrane channel, is most likely an outer membrane protein, TolC (Fralick et al., (1996) J Bacteriol, 178(19), 5803-5). Providing additional insight into the workings of the AcrAB system was the solving of the crystal structures of AcrB and TolC (see, Murakami et al., Nature, 419: 587-593, 2002 and Koronakis et al., Nature 415: 914-919, 2000).

Mutations that increase AcrAB expression increase the MICs (minimum inhibitory concentrations) of agents such as fluoroquinolones, tetracycline, chloramphenicol, novobiocin, and fusidic acid and as such the acrAB gene products and their homologues are implicated in increased drug resistance among gram negative bacterial species.

A high throughput screen used to test compounds for inhibition of efflux pump activity using alomar blue as a viability marker at subinhibitory concentrations of novobiocin in E. coli has been described. (Thorarensen et al., (1997) Bioorg Med Chem Lett 11(14), 1903-6). Compounds that inhibited efflux pump activity, caused novobiocin to accumulate and prevent growth. Although this assay provides valuable information on the identity of specific compounds as potential efflux pump inhibitors, the assay does not provide specific and detailed information about the function of the efflux pump. A compound identified as an inhibitor using the alomar blue assay may inhibit cell growth in a manner that is not directly dependent on efflux pump activity.

Ocaktan (Ocaktan et al, (1997) J Biol Chem 272(35), 21964-9) reported the use of a fluorescent assay to provide kinetic information about the MexA-MexB-OprM efflux pump in Pseudomonas aeruginosa. The marker substrate used was 1-(4-trimethylammoniumphenyl)-6-phenyl-1,3,5-hexatriene (TMA-DPH), an amphiphilic probe that is weakly fluorescent in solution but strongly fluorescent in cell membranes. Sedgwick (Sedgwick et al., (1996) Biochim Biophys Acta 1278(2), 205-212) reported the use of DMP⁺ (2-4-diemthylaminostyryl)-1-ethypyridinium cation as a marker substrate for the AcrAB antiport system. Both Ocaktan and Sedgewick, however, reported kinetic time course studies not amenable to high throughput screening. Trias et al. reported the testing of antibacterial compounds in the presence or absence of putative permeability enhancers or efflux inhibitors. (U.S. Pat. No. 5,989,832). This method was time consuming because its endpoint was growth of the bacteria in question and therefore not well suited to high throughput screening.

In light of the foregoing, a method amenable to high throughput screening for compounds capable of inhibiting the acrAB efflux pump is desired. Such a system is described herein.

### SUMMARY OF THE INVENTION

In a first aspect, this invention provides a method for screening for efflux pump inhibitors of the AcrAB system. This method comprises: contacting an outer membrane permeabilized, gram negative bacterium which expresses an AcrAB efflux pump with an AcrAB marker substrate compound and a test agent; determining the intracellular concentration or rate of accumulation of the marker substrate compound at one or more times; and comparing the intracellular concentration or rate of intracellular accumulation of the marker substrate compound at corresponding time, in the presence of the test agent with the intracellular concentration or rate of intracellular accumulation in the presence of a positive or negative control, wherein an increase compared to the negative control or a similar concentration or accumulation compared to the positive control indicates that the test agent is an inhibitor of the AcrAB efflux pump.

The invention also provides additional methodology to confirm that a given test agent is a specific inhibitor of the AcrAB efflux pump.

In another embodiment of the invention, the use of bacteria that over-express the acrAB gene products either naturally or through means that are well known in the art are provided. Such methods include, but are not limited to, the cloning of nucleic acids expressing such proteins and operative insertion into a multi-copy plasmid expression vector, or treating bacterial cells with compounds known to induce expression of the acrAB operon, or the isolation of mutant strains which overexpress the acrAB gene product(s).

### BRIEF DESCRIPTION OF THE FIGURES

FIG. 1 shows an outline of important kinetic processes and compartments relevant to the underlying mathematical analyses of data from efflux transport.

### DETAILED DESCRIPTION OF THE INVENTION

This invention provides bacterial strains and methods for screening compounds to identify acrAB efflux pump inhibitors, which are compounds that inhibit the acrAB efflux pump of Escherichia coli and other gram negative organisms. Exposing a bacterium expressing the acrAB efflux pump polypeptides to an acrAB efflux pump inhibitor can significantly slow the export of an antibacterial agent from the interior of the cell. Therefore, if another antibacterial agent is administered in conjunction with an efflux pump inhibitor, the antibacterial agent which would otherwise be maintained at a very low intracellular concentration by the export process, can accumulate to a concentration that will inhibit the growth of the bacterial cells. The growth inhibition can be due to either bacteriostatic or bactericidal effects depending on the antibacterial agent used. Therefore compounds identified by the process would have one of the following biological effects:
1. Gram negative bacteria with a functional acrAB gene product will become susceptible to antibiotics that could not be used for treatment of gram negative infections or would become more susceptible to antibiotics which do inhibit gram negative growth.
2. Gram negative bacteria with a functional acrAB gene product will become more susceptible to antibiotics currently in use for treating gram negative infections.

Obtaining even one of these effects provides a therapeutic treatment for infections with Escherichia coli or other bacteria. Also, as previously mentioned, similar pumps are found in other gram negative bacteria which have acrAB genes with similarity to the E. coli genes. Some or all of the effects outlined can be obtained with these bacteria. Further, these bacteria are also appropriate targets for detecting or using efflux pump inhibitors.

As used herein the term “AcrAB efflux pump” and “AcrAB efflux pump polypeptides” includes efflux pumps that have homology with the AcrAB efflux pump of E. coli and are expressed via transcription from a polycistronic message as exemplified by the E. coli acrAB operon and that function in concert as a transmembrane pump. “AcrAB efflux pump” and “AcrAB efflux pump polypeptides” encompass polypeptides that share structural homology and that export similar compounds. Identifying polypeptides that share structural homology with the polypeptides of the AcrAB efflux pump is readily within the skill of one of ordinary skill in the art. The molecular properties and conserved structural motifs of the AcrAB family of bacterial multidrug transporters as well as the other transporters described herein has been reviewed by Putman et al. Microbiology and Molecular Biology Reviews 64: pg 672-693 (2000)

In some embodiments, the phrase “AcrAB efflux pump polypeptides” and “AcrAB efflux pump” mean gene products having SEQ ID NOS: 2 and/or 12 or gene products having sequence similarity at least 25%, at least 30%, at least 40%, at least 50%, at least 60%, at least 70%, at least 80%, at least 90%, at least 95%, or at least 99% to SEQ ID NOS: 2 and/or 12, and are expressed via transcription from a polycistronic message as exemplified by the E. coli acrAB operon and which function in concert as a transmembrane pump. The term “similarity” is defined below. SEQ ID NOS: 2 and 12 are derived from GenBank nucleic acid accession numbers U00734 and M94248 represented by SEQ ID NOS: 1 and 11 and are represented in GenBank by protein accession numbers AAA67134 and AAA67135, respectively, and are also disclosed by Ma et al. J. Bacteriol. 175 (19), 6299-6313 (1993).

“AcrAB efflux pump” and “AcrAB efflux pump polypeptides” also encompasses substitution variants of SEQ ID NO: 2 and 12. Substitution variants are those polypeptides wherein one or more amino acid residues of an AcrAB polypeptide are removed and replaced with alternative residues. In one aspect, the substitutions are conservative in nature, however, the invention embraces substitutions that are non-conservative. Conservative substitutions are defined below. This definition of AcrA and AcrB efflux pump polypeptides therefore encompasses strain variants of Escherichia coli acrAB gene products or sequences found in Escherichia coli strains other than K12 strains. For example, Escherichia coli O157:H7 EDL933 has sequences reported for its acrAB gene products. (GenBank protein accession number AAG54812 (AcrA) and AAG54811 (AcrB) derived from nucleic acid accession numbers AE005225 and AE005174). The 0157 acrA gene product is reported to be identical to SEQ ID NO:2. The 0157 acrB gene product exhibits about 98 percent similarity to SEQ ID NO:10.

The definition of AcrAB efflux pump polypeptides, therefore encompasses variants of AcrA and AcrB disclosed in SEQ ID NOS: 4, 6, 8, 10, 14, 16, 18, 20, 65, and 67 which represent AcrAB variants from Klebsiella pneumoniae, Pseudomonas aeruginosa, Salmonella typhimurium, Enterobacter aerogenes, and Haemophilus influenza. The acrAB variant gene products of Klebsiella pneumoniae are presented in SEQ ED NO:4 and 14 and are encoded by SEQ ID NOS: 3 and 13. SEQ ID NOS: 4 and 14 are also found in GenBank with accession numbers CAC41008 (AcrA) and CAC41009 (AcrB). The AcrA homologue of Klebsiella pneumoniae exhibits about 88% similarity with SEQ ID NO:2 and the AcrB homologue of Klebsiella pneumoniae exhibits about 95% similarity with SEQ ID NO: 12. The mexAB gene products of Pseudomonas aeruginosa are presented in SEQ ID NOS:6 and 16 and are encoded by SEQ ID NOS: 5 and 15. SEQ ID NOS: 6 and 16 are also found in GenBank with accession numbers P52477 (MexA) and S39630 (MexB). The mexA gene product exhibits about 62% similarity to SEQ ID NO:2 and the mexB gene product exhibits about 79% similarity to SEQ ID NO:12. The acrAB variant gene products of Salmonella enterica are presented in SEQ ID NO:8 and 18 and are encoded by SEQ ID NOS:7 and 17. The acrA variant of Salmonella enterica exhibits about 94% similarity with SEQ ID NO:2 and the acrB variant of Salmonella enterica exhibits about 97% similarity with SEQ ID NO:12. This definition also therefore encompasses the acrAB variant gene products of Enterobacter aerogenes presented in SEQ ID NO:10 and 20 and encoded by SEQ ID NOS:9 and 19. SEQ ID NOS:10 and 20 are also found in GenBank with accession numbers CAC35724 (AcrA) and CAC35725 (AcrB). The AcrA homologue of Enterobacter aerogenes exhibits about 88% similarity with SEQ ID NO:2 and the AcrB homologue of Enterobacter aerogenes exhibits about 95% similarity with SEQ ID NO: 12. The acrAB gene products of Haemophilus influenza are presented in SEQ ID NOS: 65 and 67 and are encoded by SEQ ID NOS: 64 and 66, respectively. SEQ ID NOS: 64, 65, 66, and 67 are also found in Genbank with accession numbers U32771, AAC22554, U32771, and AAC22555, respectively. The AcrA homologue of Haemophilus influenza exhibits about 33% similarity with SEQ ID NO:2 and the AcrB homologue of Haemophilus influenza exhibits about 48% similarity with SEQ ID NO:12. The protein described in SEQ ID NOS: 65 and 67 are described as homologues of the AcrAB proteins in Sanchez et al. (J. Bact. 179, 6855-6857 (1997)).

“AcrAB efflux pump” and “AcrAB efflux pump polypeptides” also encompasses the acrEF gene products of gram negative species, including but not limited to, Escherichia coli, Pseudomonas aeruginosa and Salmonella enterica gene products presented in SEQ ID NOS: 22, 26, 28, 30, 32, 34 and 36 and which are encoded by SEQ ID NOS: 21, 25, 27, 29, 31, 33 and 35 respectively. The Escherichia coli acrA gene product shares 74% and 59% similarity with the Escherichia coli and Pseudomonas aeruginosa acrE gene product. The Escherichia coli acrB gene product shares 84%, 66% and 78% similarity with the Escherichia coli, Pseudomonas aeruginosa, Salmonella enterica acrF gene products respectively. One of ordinary skill in the art recognizes that of all of the above sequences from other enteric and gram negative species are easily obtained or identified by database mining and subsequent hybridization experiments or polymerase chain reactions. Once one of ordinary skill is in possession of the relevant acrAB sequences it becomes a matter of routine to delete or mutate or clone portions of the coding sequences to inactivate or elevate the production of the acrAB gene products.

As used herein, a “disrupted locus” means a gene which is incapable of expressing a functional gene product. For example, the coding sequence may have undergone a mutation, insertion, a deletion, or a substitution, or its regulatory sequences may have undergone a mutation, insertion, deletion, or substitution.

The phrase “an elevated concentration” of a compound refers to an intracellular concentration that indicates that the compound, generally an antibacterial agent, is at a higher concentration inside the cell in the presence of another compound, generally a test compound or an efflux pump inhibitor, than in the absence of the compound, generally a test compound or an efflux pump inhibitor. Thus, in the description of the screening methods, an elevated intracellular concentration of an antibacterial agent is at a concentration higher than that existing in the absence of the test compound or of a known efflux pump inhibitor. This elevated concentration may be lower, the same as, or higher than the concentration existing in the medium.

As used herein the phrase “emrD locus” means DNA sequences which encode polypeptides with similarity to SEQ ID NO: 46 and which are bacterial proteins that comprise 14 transmembrane domains and that are members of subfamily I of the major facilitator superfamily of drug resistance proteins and which export similar compounds as the EmrD polypeptide that is disclosed in SEQ ID NO:46. This definition encompasses DNA sequence(s) that comprise sequence(s) having at least 40%, at least 50%, at least 60%, at least 70%, at least 80%, at least 90%, at least 95%, at least 97%, at least 99%, or at least 100% similarity to SEQ ID NO:45, and which encode EmrD polypeptides having at least 40%, at least 50%, at least 60%, at least 70%, at least 80%, at least 90%, at least 95%, at least 97%, at least 99%, or at least 100% similarity to SEQ ID NO:46. The emrD coding sequence of Escherichia coli is disclosed in SEQ ID NO:45 and the encoded amino acid sequence is disclosed in SEQ ID NO:46. This definition encompasses loci which encode substitution variants of SEQ ID NO:46. Substitution variants are those polypeptides wherein one or more amino acid residues of EmrD polypeptide are removed and replaced with alternative residues. In one aspect, the substitutions are conservative in nature, however, the invention embraces substitutions that are non-conservative. Conservative substitutions are defined below.

The definition of emrD loci encompasses those loci that encode polypeptides which are variants of EmrD disclosed in SEQ ID NOS: 46, 50 and 52, reflecting EmrD variants from Escherichia coli, Pseudomonas aeruginosa and Salmonella enterica, respectively, as well as strain variants of any of the sequences disclosed. The emrD coding sequence of Pseudomonas aeruginosa is disclosed in SEQ ID NO:49 and the encoded amino acid sequence is disclosed in SEQ ID NO:50. The emrD coding sequence of Salmonella enterica is disclosed in SEQ ID NO:51 and the encoded amino acid sequence is disclosed in SEQ ID NO:52. The Escherichia coli and Pseudomonas aeruginosa emrD loci exhibit about 50 percent similarity at the DNA level and exhibit about 46 percent similarity at the protein level. The Escherichia coli and Salmonella enterica emrD loci exhibit about 83 percent similarity at the DNA level and exhibit about 96 percent similarity at the protein level. One of ordinary skill in the art recognizes that the sequence of variants from other enteric and gram negative species are easily obtained or identified by database mining and subsequent hybridization experiments or polymerase chain reactions. Once one of ordinary skill is in possession of the relevant emrD sequence it becomes a routine matter to delete or mutate portions of the coding sequence to inactivate the production of the emrD gene product. As used herein a “disrupted emrD locus” means an emrD locus which is incapable of expressing a functional gene product. For example, the coding sequence may have undergone a mutation, insertion, deletion or substitution, or its regulatory sequences may have undergone a mutation, insertion, deletion, or substitution.

As used herein, the phrase “emrE locus” means DNA sequence(s) that encode polypeptides with similarity to SEQ ID NO:37 and which are bacterial proteins which comprise transmembrane domains and are members of the small multidrug resistance family of resistance proteins. This definition encompasses DNA sequence(s) which comprise sequence(s) having at least 50%, at least 60%, at least 70%, at least 80%, at least 90%, at least 95%, at least 97%, at least 99%, or at least 100% similarity to SEQ ID NO: 37 and that encode EmrE polypeptides having at least 50%, at least 60%, at least 70%, at least 80%, at least 90%, at least 95%, at least 97%, at least 99%, or at least 100% similarity to SEQ ID NO:38 The emrE coding-sequence of Escherichia coli is disclosed in SEQ ID NO:37 and the encoded amino acid sequence is disclosed in SEQ ID NO:38.

The “emrE locus” also encompass loci that encode substitution variants of SEQ ID NO: 38. Substitution variants are those polypeptides wherein one or more amino acid residues of emrE polypeptide are removed and replaced with alternative residues. In one aspect, the substitutions are conservative in nature, however, the invention embraces substitutions that are non-conservative. Conservative substitutions are defined below.

emrE loci encompass those loci that encode polypeptides which are variants of EmrE disclosed in SEQ ID NOS: 38, 42 and 44, reflecting EmrE variants from Escherichia coli, Pseudomonas aeruginosa and Salmonella enterica, respectively, as well as strain variants of any of the sequences disclosed. The emrE coding sequence of Pseudomonas aeruginosa is disclosed in SEQ ID NO:41 and the encoded amino acid sequence is disclosed in SEQ ID NO:42. The emrE coding sequence of Salmonella typhimurium is disclosed in SEQ ID NO:43 and the encoded amino acid sequence is disclosed in SEQ ID NO:44. The Escherichia coli and Pseudomonas aeruginosa emrE loci exhibit about 53 percent similarity at the DNA level and exhibit about 68 percent similarity at the protein level. The Escherichia coli and Salmonella enterica emrE loci exhibit about 56 percent similarity at the DNA level and exhibit about 69 percent similarity at the protein level. One of ordinary skill in the art recognizes that the sequence of variants from other enteric and gram negative species are easily obtained or identified by database mining and subsequent hybridization experiments or polymerase chain reactions. Once one of ordinary skill is in possession of the relevant emrE sequence, it becomes a routine matter to delete or mutate portions of the coding sequence to inactivate the production of the emrE gene product. As used herein a “disrupted emrE locus” means an emrE locus which is incapable of expressing a functional gene product. For example, its coding sequence may have undergone a mutation, insertion, deletion, or substitution, or its regulatory sequences may have undergone a mutation, insertion, deletion, or substitution.

As used herein, the phrase “enteric bacteria” refers to any genus, species, subspecies, serotype, strain, or any other taxonomic designation attributed to any member of the Enterobacteriaceac. It is not intended that the term be limited to any particular genus or species.

The term “expression” or “expressing” of a gene refers to the cellular processes of transcription and translation to produce a polypeptide product. In this context, the term further implies that the expression product is functional, in the sense that it is readily detectable by the means appropriate for that specific reporter. Thus, for a transport protein the product exhibits the normal transport activity.

The phrase “expressing at a reduced levels” means expressing a gene product at a level reduced from the levels of expression in a comparator strain. This definition would include no expression. Therefore a strain with a disrupted acrAB locus and no corresponding compensating expression from an acrAB expressing plasmid would be said to be a strain expressing AcrAB at a reduced level.

The phrase “gram negative bacteria” is art recognized and is intended to include those bacteria which, when treated with either gentian violet, or its analog crystal violet, followed by iodine and an organic wash, do not stain. Typically, a counterstain of some contrasting color is applied to demonstrate gram negative bacteria. The term “gram negative bacteria” includes, but is not limited to, the following list of species of particular relevance to human and animal health: Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii and Bacteroides splanchnicus.

Similarity and sequences identified by percent similarity with other sequences are referred to in this document. In general, these terms have meanings generally accepted by one skilled in the art. To calculate a percent sequence similarity, one must first align the relevant sequences. Alignments of sequences and calculation of identity/similarity scores were done using a Clustal W (J. D. Thompson et al (1994) NAR 22 (22) p4673-4680) alignment, useful for both protein and DNA alignments. The default scoring matrices Blosum62mt2 and swgapdnamt are used for protein and DNA alignments respectively. The gap opening penalty is 10 and the gap extension penalty: 0.1 for proteins. The gap opening penalty is 15 and the gap extension penalty is 6.66 for DNA. The alignments can be done using the computer program alignX which is a component of the Vector NTI Suite 7.0 package from Informax, Inc (www.informax.com) using the default settings. Described herewith are two definitions of similarity, one in reference to DNA or nucleic acid sequences and one in reference to peptide or protein sequences.

Nucleic acid similarity: With respect to nucleic acids, the terms “identity” and “similarity” are synonymous. A first nucleic acid sequence exhibits X % identity/similarity to that of a second nucleic acid sequence when the first nucleic acid sequence contains after alignment, at any point within the sequence, X nucleotide bases out of 100 which are identical to that of the second nucleic acid sequence. As noted above using the computer program alignX from Informax, Inc is a representative way to easily and quickly calculate percent sequence identity of nucleic acids.

Percent amino acid sequence “identity” with respect to polypeptides is defined herein as the percentage of amino acid residues in the candidate sequence that are identical with the residues in the target sequences after aligning both sequences and introducing gaps, if necessary, to achieve the maximum percent sequence identity, and not considering any conservative substitutions as part of the sequence identity. Percent sequence identity is determined by conventional methods (See, for example, Henikoff and Henikoff, Proc. Nat. Acad. Sci. USA 8:10915-10919, 1992). Briefly, as noted above two amino acid sequences are aligned to optimize the alignment scores using a gap opening penalty of 10, a gap extension penalty of 0.1, and modification (blosum62mt2) of the “blosum62” scoring matrix of Henikoff and Henikoff (ibid.) as shown in Table 1 (amino acids are indicated by the standard one-letter codes).

TABLE 1

| | A | C | D | E | F | G | H | I | K | L | M | N | P | Q | R | S | T | V | W | Y |
|---|---|---|---|---|---|---|---|---|---|---|---|---|---|---|---|---|---|---|---|---|
| A | 8 | | | | | | | | | | | | | | | | | | | |
| C | 0 | 18 | | | | | | | | | | | | | | | | | | |
| D | −4 | −6 | 12 | | | | | | | | | | | | | | | | | |
| E | −2 | −8 | 4 | 10 | | | | | | | | | | | | | | | | |
| F | −4 | −4 | −6 | −6 | 12 | | | | | | | | | | | | | | | |
| G | 0 | −6 | −2 | −4 | −6 | 12 | | | | | | | | | | | | | | |
| H | −4 | −6 | −2 | 0 | −2 | −4 | 16 | | | | | | | | | | | | | |
| I | −2 | −2 | −6 | −6 | 0 | −8 | −6 | 8 | | | | | | | | | | | | |
| K | −2 | −6 | −2 | 2 | −6 | −4 | −2 | −6 | 10 | | | | | | | | | | | |
| L | −2 | −2 | −8 | −6 | 0 | −8 | −6 | 4 | −4 | 8 | | | | | | | | | | |
| M | −2 | −2 | −6 | −4 | 0 | −6 | −4 | 2 | −2 | 4 | 10 | | | | | | | | | |
| N | −4 | −6 | 2 | 0 | −6 | 0 | 2 | −6 | 0 | −6 | −4 | 12 | | | | | | | | |
| P | −2 | −6 | −2 | −2 | −8 | −4 | −4 | −6 | −2 | −6 | −4 | −4 | 14 | | | | | | | |
| Q | −2 | −6 | 0 | 4 | −6 | −4 | 0 | −6 | 2 | −4 | 0 | 0 | −2 | 10 | | | | | | |
| R | −2 | −6 | −4 | 0 | −6 | −4 | 0 | −6 | 4 | −4 | −2 | 0 | −4 | 2 | 10 | | | | | |
| S | 2 | −2 | 0 | 0 | −4 | 0 | −2 | −4 | 0 | −4 | −2 | 2 | −2 | 0 | −2 | 8 | | | | |
| T | 0 | −2 | −2 | −2 | −4 | −4 | −4 | −2 | −2 | −2 | −2 | 0 | −2 | −2 | −2 | 2 | 10 | | | |
| V | 0 | −2 | −6 | −4 | −2 | −6 | −6 | 6 | −4 | 2 | 2 | −6 | −4 | −4 | −6 | −4 | 0 | 8 | | |
| W | −6 | −4 | −8 | −6 | 2 | −4 | −4 | −6 | −6 | −4 | −2 | −8 | −8 | −4 | −6 | −6 | −4 | −6 | 22 | |
| Y | −4 | −4 | −6 | −4 | 6 | −6 | 4 | −2 | −4 | −2 | −2 | −4 | −6 | −2 | −4 | −4 | −4 | −2 | −2 | 14 |

The percent identity is then calculated as:

$$\frac{Total number of identical matches}{\begin{matrix}[ length of the longer sequence + number of gaps \\ introduced into the longer sequence to align \\ the two sequences ]\end{matrix}} \times 100$$

Percent sequence “similarity” (often referred to as “homology”) with respect to the preferred polypeptide of the invention is defined herein as the percentage of amino acid residues in the candidate sequence that are identical with the residues in the target sequences after aligning the sequences and introducing gaps, if necessary, to achieve the maximum percent sequence identity (as described above) and also considering any conservative substitutions as part of the sequence identity.

$$\frac{Total number of identical matches and conservative substitutions}{\begin{matrix}[ length of the longer sequence + number of gaps \\ introduced into the longer sequence to align \\ the two sequences ]\end{matrix}} \times 100$$

Amino acids can be classified according to physical properties and contribution to secondary and tertiary protein structure. A conservative substitution is recognized in the art as a substitution of one amino acid for another amino acid that has similar properties.

Exemplary conservative substitutions used as default values in the Vector NTI Suite, AlignX program are set out in Table 2.

TABLE 2

| Amino Acid | Conservative Changes |
|---|---|
| Alanine (A) | Glycine (G), Serine (S) |
| Aspartic Acid (D) | Glutamic Acid (E) |
| Glutamic Acid (E) | Aspartic Acid (D) |
| Phenylalanine (F) | Tryptophan (W), Tyrosine (Y) |
| Glycine (G) | Alanine (A) |
| Histidine (H) | Tyrosine (Y) |
| Isoleucine (I) | Leucine (L), Methionine (M), Valine (V) |
| Lysine (K) | Arginine (R) |
| Leucine (L) | Isoleucine (l), Methionine (M) Valine (V) |
| Methionine (M) | Isoleucine (l), Leucine (L), Valine (V) |
| Asparagine (N) | Glutamine (Q) |
| Glutamine (Q) | Asparagine (N) |
| Arginine (R) | Lysine (K) |
| Serine (S) | Alanine (A), Threonine (T) |
| Threonine (T) | Serine (S) |
| Valine (V) | Isoleucine (I), Methionine (M) Valine (V) |
| Tryptophan (W) | Phenylalanine (F), Tyrosine (Y) |
| Tyrosine (Y) | Phenylalanine (F) Histidine (H) Tryptophan (W) |

The term “isogenic” refers to one or more bacterial strains which have the same genotypes. A strain can be said to be “isogenic but for” the expression of a particular gene product. For example, a strain that has the genotype ΔacrEF, ΔemrE, ΔemrD, ΔwaaP is said to be isogenic with a strain having the genotype ΔacrAB, ΔacrEF, ΔemrE, ΔemrD, ΔwaaP but for the expression of acrAB. The strain having the genotype ΔacrAB, ΔacrEF, ΔemrE, ΔemrD would also be said to be “expressing acrAB at reduced levels” relative to the strain having the genotype ΔacrEF, ΔemrE, ΔemrD, ΔwaaP as described in the definition of “expressed at reduced levels” above. A strain whose genotype is described as ΔacrAB, ΔacrEF, ΔemrE, ΔemrD, ΔwaaP [acrAB] (with acrAB expressed on a plasmid) is said to be isogenic with a strain having the genotype ΔacrAB, ΔacrEF, ΔemrE, ΔemrD, ΔwaaP “but for” the expression of acrAB or might be said to be isogenic but for the expression of acrAB on a plasmid.

As used herein, the phrase “marker substrate compound” means a compound capable of being transported by the AcrAB efflux pump, preferably one whose intracellular concentration can be easily determined.

As used herein, the phrase “outer membrane permeabilized gram negative bacteria” means a gram negative bacteria having an outer membrane that has been disrupted or structurally compromised either via mutation of the genetic material encoding proteins responsible for contributing to outer membrane function or via exogenous treatment to directly alter the structural integrity of the outer membrane.

As used herein, the phrase “single copy” indicates that the nucleotide sequence to which the phrase refers is present in only a single copy in each chromosome set. This is specifically distinguished from the presence of the nucleotide sequence in multi-copy plasmids, where the sequence would be present in a single cell in varying numbers greater than one. Also in reference to the nucleotide sequence inserted in a cell, the phrase “chromosomally residing” indicates that the sequence is covalently linked in the bacterial chromosome. This implies that the sequence will be replicated along with the rest of the chromosome in the normal cellular replication process. Again, this is specifically distinguished from having a nucleotide sequence present in a plasmid within the cell which is independent of the bacterial chromosome.

As used herein, the phrase “test agent” means a compound that is assessed for its ability to inhibit the AcrAB efflux pump.

The phrase “uncoupling compound” means substances that promote the passage of hydrogen (H⁺) ions across the membrane, causing dissipation of the proton-motive force. One uncoupling compound is carbonyl cyanide m-chlorophenylhydrazone. Other uncoupling compounds, include but are not limited to, proton carriers such as nitro-, halo- and oxygenated phenols and carbonylcyanide phenylhydrazones. Preferred of such proton carriers are carbonylcyanide, p-trifluoromethoxyphenylhydrazone (FCCP), carbonylcyanide M-chlorophenylhydrazone (CCCP), carbonylcyanide phenylhydrazine (CCP), tetrachloro-2-trifluoromethyl benzimidazole (TTFB), 5,6-dichloro-2-trifluoromethyl benzimidazole (DTFB), and Uncoupler 1799.

As used herein the phrase “waaP locus” means DNA sequence(s) that encode WaaP polypeptides that are expressed via transcription from a polycistronic waa gene cluster containing genes involved in the synthesis and assembly of core lipopolysacharide. Such clusters are exemplified in the disclosure of WO98/00395.

The “waaP locus” definition therefore encompasses DNA sequence(s) that comprise SEQ ID NO:53 and/or sequences having at least 50%, at least 60%, at least 70%, at least 80%, at least 90%, at least 95%, at least 97%, or at least 99% similarity to SEQ ID NO: 53 and encode waaP polypeptides having SEQ ID NO:54 or having at least 50%, at least 60%, at least 70%, at least 80%, at least 90%, at least 95%, at least 97%, at least 99% similarity to SEQ ID NO:54 and are expressed via transcription from a polycistronic waa gene cluster containing genes involved in the synthesis and assembly of core lipopolysacharide. Such clusters are exemplified in the disclosure of WO98/00395.

The gene product of the waaP locus specifically phosphorylates an inner-core heptose residue of lipolysaccharide. The waaP coding sequence of Escherichia coli is disclosed in SEQ ID NO:53 and the encoded amino acid sequence is disclosed in SEQ ID NO:54. This definition also encompasses loci that encode substitution variants of SEQ ID NO: 54. Substitution variants are those polypeptides wherein one or more amino acid residues of waaP polypeptide are removed and replaced with alternative residue. In one aspect, the substitutions are conservative in nature, however, the invention embraces substitutions that are non-conservative. Conservative substitutions are defined above. The definition of WaaP polypeptides therefore encompasses variants of WaaP disclosed in SEQ ID NOS: 56 and 58 reflecting WaaP variants from Pseudomonas aeruginosa and Salmonella typhimurium respectively as well as strain variants of any of the sequences disclosed. The waaP coding sequence of Pseudomonas aeruginosa is disclosed in SEQ ID NO:55 and the encoded amino acid sequence is disclosed in SEQ ID NO:56. The Escherichia coli and Pseudomonas aeruginosa WaaP proteins exhibit about 51 similarity at the DNA level and exhibit about 65 percent similarity at the protein level. The waaP coding sequence of Salmonella typhimurium is disclosed in SEQ ID NO:57 and the encoded amino acid sequence is disclosed in SEQ ED NO:58. One of ordinary skill in the art recognizes that variants from other enteric and gram negative species are easily obtained or identified by database mining and subsequent hybridization experiments or polymerase chain reactions. Once one of ordinary skill is in possession of the relevant waaP sequence it becomes a matter of routine to delete or mutate portions of the coding sequence to inactivate the production of the waaP gene product. As used herein, a “disrupted waaP locus” means a waaP locus which is incapable of expressing a functional gene product. For example the coding sequence may have undergone a mutation, insertion, deletion, or substitution, or its regulatory sequences may have undergone a mutation, insertion, deletion, or substitution.

The term “wildtype” means gram negative bacteria expressing non disrupted functional acrAB, acrEF, emrD and emrE gene products. In addition, a bacterial strain can be said to be “wildtype” for any locus which is not disrupted.

As used herein, the phrase “positive control” refers to a compound or compounds that are known to be AcrAB efflux pump inhibitors.

As used herein, the phrase “negative control” may refer to a compound that is not an AcrAB efflux pump inhibitor. In some embodiments, “negative control” refers to a solution that contains no compound at all. In some embodiments, “negative control” refers to a compound or compounds that are known to not be efflux pump inhibitors. For example, in an experiment where a negative control is used it may be just that the test agent is not added.

The invention comprises a number of bacterial strains, including those that are discussed below. The invention further comprises screening assays for use in selection of putative acrAB inhibitors, including those discussed below. The invention also comprises methods having optional additional steps to confirm that a test compound is likely to be a specific AcrAB inhibitor.

The Bacterial Strains of the Invention

A. Permeabilized Strains of Bacteria

The outer membrane of gram-negative bacteria such as E. coli limits the uptake of molecules above a certain size threshold. It was recognized that this might impact on uptake of putative efflux inhibitors that might be too large to enter the cell across the outer membrane, resulting in a failure to identify molecules that might, in fact, be good efflux inhibitors. We have discovered that outer membrane permeabilization is desirable when attempting to assess the effectiveness of compounds as inhibitors of the AcrAB system. If candidate test compounds have desirable characteristics as true AcrAB efflux pump inhibitors it may then be possible to make modifications to the compound to arrive at a structure which is able to pass the outer membrane.

Outer membrane permeabilization can be accomplished by treating the cells with chelating agents like EDTA or EGTA or the use of mutants in loci known to affect outer membrane integrity. Various other mutations of E. coli are known to affect the permeability of the outer membrane. These include mutant alleles of rfa (Ames et al., (1973) PNAS, 70(8), 2281-5), envA (Young et al. (1991) J Bacteriol 173(12), 3609-14), imp (Sampson et al., (1989) Genetics 122(3), 491-501), 1pp (Gi am et al., (1984) J Biol Chem, 259(9), 5601-5) or surA (Tormo et al., (1990) J Bacteriol 172(8), 4339-47).

One outer membrane permeability enhancing target is the waaP locus in E. coli and homologs in enteric bacteria and other gram negative bacteria. The term “waaP locus” is defined above. Also included are deletion or insertion mutants because such mutations limit the occurrence of reversions and therefore make the mutant strain more “stable.” The construction of a deletion mutants of the waaP locus is detailed in Example 1.

B. Multiple Pump Deleted Strains of Gram Negative Bacteria

We have found that ethidium bromide is a good substrate for the AcrAB transporter in E. coli. There is very little accumulation of ethidium bromide in wild type cells. However, following treatment of the wild type cells with uncoupling agents which inhibit efflux by disrupting the proton motive force required for activity—a significant increase in accumulation of ethidium bromide is detected. This indicates a loss of efflux pump activity in the presence of the uncoupling compounds. We also evaluated the accumulation of ethidium bromide in acrAB⁻ E. coli. The acrAB⁻ strain accumulated significantly more ethidium bromide than the wild type strain, as expected. However, it is interesting to note that the increase in accumulation of ethidium bromide in the acrAB ⁻ strain is not as high as in the uncoupler treated wild type strain. This suggests that the AcrAB pump is not the only pump acting on ethidium bromide. Moreover, uncoupler treatment significantly increased ethidium bromide accumulation in the acrAB⁻ cells, strongly supporting the idea that ethidium bromide is pumped, not only by the AcrAB pump, but also by another energy-dependent efflux pump system type cells. Because of the multifactorial nature of transport of ethidium bromide, we set out to construct a strain deleted for its other known or suspected transporters. The strategy employed is detailed below. We postulated that a strain with even acrAB deleted would be a valuable tool in ascertaining whether test compounds were having an effect mediated through the acrAB gene product or through some non-specific mechanism. Such a deletion strain can have the expression of any deletion loci reconstituted by simple reintroduction of the gene into the chromosome or via plasmid based expression. We chose to construct a strain with all four deletions and to reconstitute the expression of the AcrAB pump. Such an expression scheme has advantages because the acrAB gene products can be over-expressed via introduction on a multicopy plasmid. The details of the construction of the quadruple deletion is outlined in Example 2.

C. DNA Methodology

Basic DNA procedures, including restriction endonuclease digestions, ligations, transformations and agarose gel electrophoresis were performed as described in Sambrook et al. (1989). The alkaline lysis method (Sambrook et al., 1989) or a plasmid midi kit (Qiagen Inc.) was used to isolate plasmids from E. coli DH5α. The genomic DNA of E. coli was extracted by the method of Barcack et al. (1991). DNA fragments used in cloning were extracted from agarose gels using Prep-A-Gene (Bio-Rad Labs, Richmond, Calif.) as per the manufacturer's instructions. E. coli cells were made competent using the CaCl₂ method (Sambrook et al., 1989) or, when super-competent cells of E. coli were required, the method of Inoue et al. (1991). Electro-competent cells of E. coli were prepared from harvesting mid-log phase cells grown in LB broth, washing two times with chilled H₂O and once with chilled 10% (vol/vol) glycerol, and resuspending in 10% (vol/vol) glycerol. Nucleotide sequencing of plasmid-bome DNA was carried out by Cortec DNA Services Inc. (Queen's University, Kingston, Ontario, Canada) using universal or custom primers. Compilation of DNA sequence data was performed using DNAMAN (Version 4.11, Lynnon Biosoftware, Vaudreuil, Quebec, Canada).

Strains, Expressing acrAB on a Multicopy Plasmid

One aspect of the invention encompasses bacterial strains (and assays that utilize these strains) in which acrAB gene products are overexpressed. As one means of accomplishing this we set out to express these products in plasmid expression systems in the strain which had the chromosomal acrAB expression disrupted.

Assays to Identify Putative Inhibitors of AcrAB

The invention also comprises assays to identify putative inhibitors of AcrAB. In one embodiment, the invention contemplates exposing permeabilized bacterial cells expressing the acrAB gene products to a marker substrate compound transported by the acrAB gene product and allowing the system to equilibrate such that inward and outward fluxes of substrate into the bacterial cell reach a steady state (i.e., the interior concentration within the cell reaches a constant value) The concentration within the cell is then measured. Such an equilibration experiment is performed either sequentially or contemporaneously in the presence of a test compound. A test compound which is able to inhibit the acrAB efflux pump polypeptide mediated transport results in a higher steady state concentration within the cells incubated in the presence of the test compound than in its absence.

The invention also contemplates exposing permeabilized bacterial cells expressing the acrAB gene products to a marker substrate compound transported by the acrAB gene product. The concentration within the cell is measured one time or multiple times such that the rate of internal accumulation can be determined. Such an experiment wherein the rate is calculated is performed either sequentially or contemporaneously in the presence of a test compound. A test compound which is able to inhibit the AcrAB efflux pump polypeptide mediated transport results in a higher rate of accumulation with the cells incubated in the presence of the test compound than in its absence. It is appreciated that in such an analysis that the measurement of initial rates are preferred.

The marker substrate can be, for example, a fluorescent compound that changes it spectroscopic properties upon entering the cell. Typically these compounds fluoresce weakly in aqueous environments, but become strongly fluorescent in non-polar or hydrophobic environments. The invention of course is not limited to compounds exhibiting these properties. While it is well known that fluorescent substrates provide a convenient method of estimating intracellular concentrations of a substrate, other compounds are suitable as well. These compounds include, but are not limited to, radioactively labeled compounds. Fluorescent compounds that can be used in the invention include intercalating compounds including, but not limited to, ethidium bromide, acridine orange, or proflavin. Also useful are lipophilic membrane bound dyes which change their spectral characteristics in response to the polarity of their environment. Lipophilic membrane bound dyes that can be used include, but are not limited to, amphiphilic probes that are weakly fluorescent in solution but strongly fluorescent in cell membranes such as 1-(4-trimethylammoniumphenyl)-6-phenyl-1,3,5-hexatrine (TMA-DPH), 1,6-Diphenyl-1,3,5-hexatriene (DPH), or TMAP-DPH, which incorporates an additional three-carbon spacer between the fluorophore and the trimethylammonium substituent. The term membrane bound dyes also includes dapoxyl derivatives, anilinonaphthalenesulfonate (ANS) and derivatives and polar and non-polar BODIPY derivatives.

Ethidium bromide has an excitation maximum for fluorescence of 530 mm and peak emission occurs at 600 nm, making its spectral properties less prone to interference by compounds in the usual organic compound collection than some lipophilic membrane bound dyes. Because of the desirability of ethidium bromide as a substrate for AcrAB and because we discovered that its transport is significantly multifactorial, we constructed an E. coli strain which expresses only acrAB and not any other known or suspected ethidium exporters. As noted above, it is possible to engineer a concomitant mutation in the waaP locus to obtain a strain with increased outer membrane permeability in such a genetic background. The acrAB, acrEF, emrE and emrD loci were targeted for elimination with the subsequent reconstitution of acrAB expression. The terms acrAB, acrEF, emrE and emrD loci and polypeptides and encoding nucleic acids are defined above. One of ordinary skill, however, will recognize that such elimination of every possible ethidium bromide transporter is not entirely necessary to practice the methods of the invention, but only provides additional sensitivity for assays which utilize ethidium bromide as a marker substrate compound.

The methods of this invention are suitable and useful for screening a variety of sources for possible activity as AcrAB efflux pump inhibitors. Initial screens have been performed using a diverse library of compounds, but the methods are also suitable for other compound libraries. Such libraries can be natural product libraries, combinatorial libraries, or other small molecule libraries. In addition, compounds from commercial sources can be tested; this testing is particularly appropriate for commercially available analogs of identified efflux pump inhibitors. Compounds with identified structures can be efficiently screened for efflux pump activity by first restricting the compounds to be screened to those with preferred structural characteristics.

One of skill in the art recognizes that the bacterial strains constructed have great utility in assessing the compounds capable of inhibiting the acrAB efflux pump. We set out to devise a method for assessing test compounds for their ability to inhibit the acrAB pump in E. coli.

Briefly, the method comprises, contacting an acrAB marker substrate compound with an outer membrane permeabilized, gram negative bacterium which expresses an acrAB efflux pump and incubating said marker substrate compound and said gram negative bacterium in the presence of and/or absence of a test agent and determining the intracellular concentration of said marker substrate compound. In some embodiments, the gram negative bacterium that are in the presence of a test agent are different gram negative bacterium of the same species that are not in the presence of the test agent.

D. Determination of the Specificity of a Test Compound

The invention also comprises determining the specificity of a test compound. It will be recognized that the initial screen described above will eliminate a huge number of potential test compounds and is very valuable for that purpose. It is often desirable, however, to perform additional testing steps to determine whether a compound is a specific acrAB inhibitor compound.

To determine whether a compound is a specific inhibitor of the acrAB efflux pump a number of procedures may be followed.

One method of ascertaining specificity (the “plus/minus approach”) is to incubate a test compound with multiple strains either expressing AcrAB (the comparator strain) or expressing AcrAB at a reduced level. A specific test compound would be expected to increase the intracellular concentration of the marker substrate compound more in the comparator strain than in the strain expressing the AcrAB pump at reduced levels. It is often preferred, to simplify analysis, to use a strain which has a disrupted acrAB locus. By judicious selection of strains one can also surmise whether the primary mode of action is membrane permeabilization. The data from such experiments can be optionally analyzed kinetically to add further refinement to the method.

One can envision that one class of compounds which would result in higher steady state intracellular levels of the marker substrate compound would be compounds which permeabilize animal cells as well. Determining whether a test agent is acting to effect membrane permeability can be carried out by methods well in known in the art which determine if cells become “leaky” after treatment. These are referred to as “direct assays of membrane integrity”.

Other methods of assessing whether a compound is acting specifically at the acrAB pump are “active efflux methods” which essentially involve preloading cells with a marker substrate compound and measuring efflux from the cell directly.

Below, we describe further and provide specific non-limiting examples of how one might determine whether a test compound is a specific acrAB efflux pump inhibitor.

Directly Measuring Membrane Integrity

Membrane permeabilizers are likely not compounds which would be efficacious for therapeutic use because compounds which permeabilize membranes non-specifically are potentially likely to be toxic.

One method of determining whether a compound is increasing accumulation of marker substrate compound primarily via membrane effects comprises performing assays well known in the art to directly determine whether a compound affects the intrinsic membrane integrity of the bacterial cell (“Direct Assays of Membrane Integrity”). Such assays typically involve determining the extent of “leakage” of normal cellular constituents from the periplasm or cytoplasm of the bacterial cells in response to treatment with a compound. Typically, the constituent is a protein (but need not be) and the presence or absence of the constituent is easily assayed. By way of illustration some examples are detailed below. Other examples are well known in the art.

### EXAMPLES

### Example 1

Construction of waaP Mutants.

To overcome the outer membrane barrier and facilitate the entry of putative efflux pump inhibitors into the intended screening strain(s) of E. coli, mutants carrying in-frame deletion of the waaP gene were constructed from E. coli AG100 and derivatives (i.e. AG100 ΔacrAB or AG100 ΔacrAB ΔacrEF ΔemrE ΔemrD).

The method of Link et al (Link et al., (1997) J. Bacteriol. 179(20), 6228-37) was used for construction of waaP mutants of E. coli, again using PCR to amplify sequences 5′ and 3′ to the deletion endpoints in waaP followed by their cloning into the gene replacement vector pK03. Sequence 5′ to waaP were amplified with primers (designed using the E. coli genome sequence database—GenBank accession number AE000400) waap1xz SEQ ID NO: 59 (5′-ATTCGGATCCTAAGATGCCTGGCCTGGATG-3′; BamHI site bold; anneals 854-835 bp upstream of the waaP start codon) and waap2xz SEQ ID NO:60 (5′-TCACGAATTCACGACGAGTCTCCAGTTCAC-3′; EcoRI site bold; anneals 108-89 bp downstream of the waaP start codon), while sequence 3′ to waaP were amplified with—primers waap3xz SEQ ID NO: 61 (5′-ATCCGAATTCAACATGGCAAGCGTTAAGG-3′; EcoRI bold; anneals 60-42 bp upstream of the waaP stop codon) and waap4xz SEQ ID NO: 62 (5′-ATCCGTCGACCGAAGAGTCCAGCCAGATTG-3′; SalI site bold; anneals 743-724 bp downstream of the waaP stop codon). The PCR reactions were formulated and processed as above using the waaP primers in place of the efflux gene primers. The PCR product corresponding to sequence 5′ to the waaP deletion was digested with BamHI and EcoRI and cloned into BamHI-EcoRI-restricted pBluescript II SK(+) to produce pXZLI012. The PCR product corresponding to sequence 3′ to the waaP deletion was digested with EcoRI-SalI and cloned into EcoRI-SalI-restricted pXZL1012 to produce pXZL1020. A DNA fragment carrying the waaP deletion (630 bp in-frame deletion) was subsequently released from pXZLI020 following digestion with BamHI and SalI and cloned into BamHI-SalI-restricted pK03, to produce pXZL1024. This vector was then electroporated into various E. coli strains and waaP deletions selected exactly as above for pump deletions. Screening of ΔwaaP mutants was initially carried out on LB agar plates supplemented with novobiocin (20, 5, or 2 μg/ml). Those mutants showing an increasing susceptibility to novobiocin (expected phenotype for ΔwaaP mutants) were further examined by PCR amplification of the waaP gene with primers waap1xz (see above) and waap7xz SEQ ID NO: 63 (5′-AATACGCTCGGCCTTAAC-3′; anneals 48-31 bp upstream of the waaP stop codon) to confirm the waaP deletion.

In strains which still express acrAB or have expression reconstituted but are deleted for waaP acrAB-dependent antimicrobial resistance and ethidium bromide exclusion are still observed.

### Example 2

Construction of ΔacrAB, ΔacrEF, ΔemrE, ΔemrD E. coli Strain (Δquad)

General Strategy

The acrAB, acrEF, emrE, and emrD loci were targeted for disruption with the subsequent reconstitution of acrAB expression (although the quadruple deleted strain which does not express acrAB is a useful control). A strain lacking acrAB acrEF, emrE, and emrD (dubbed XZL992) was ultimately constructed and shown to be both ethidium bromide susceptible and to lack ethidium bromide efflux activity. The acrAB genes were then engineered into this strain on plasmids permitting either constitutive (pXZL) or inducible (pXZL962) high-level expression of the genes. Expression of acrAB was subsequently confirmed and shown to promote enhanced resistance to and exclusion of ethidium bromide. These strains, thus, promoted acrAB-specific ethidium bromide efflux.

E. coli possess multiple efflux systems able to accomplish active efflux of ethidium bromide, including AcrEF, AcrAB, EmrE, EmrD. The method of Link et al (J. Bacteriol. 1997, 179: 6228-6237) was used for construction of markerless deletion mutants of the various efflux pumps, alone and in combination. Initially, PCR was used to amplify 5′ end (as N-terminal portion) and 3′ end (as C-terminal portion) of emrE, and emrD, of E. coli. Four primers (two primers for 5′ end with BamHI site and EcoRI site included in either end, respectively; two primers for 3′ end with EcoRI and SalI site included in either end, respectively) for each gene were designed based on the E. coli genome sequence data. Chromosomal DNA of K1537 (E. coli AG100 ΔacrAB ΔacrEF) was used as template.

Generally, 5′end and 3′end of the PCR products were purified and digested with BamHI and EcoRI or EcoRI and SalI. These fragments were together or individually cloned into pBluescript II SK(+) digested with BamHI and SalI (three fragment ligation) or BamHI and EcoRI or EcoRI and SalI. The cloning was then confirmed by restriction digestion and DNA sequencing. The desired BamHI-SalI fragments with appropriate deletions were then cloned into BamHI-SalI restricted pK03, a gene replacement vector that contains a temperature-sensitive origin of replication and markers for positive and negative selection for chromosomal integration and excision. Subsequent transformants were obtained at 30° C. on 20 μg/ml chloramphenicol LB agar. pK03-derivated plasmids were prepared by Qiagem™ DNA midi-kit and electroporated into electro-competent E. coli host cells (KI537 or its derivatives such as XZL986 CK1537 ΔemrE). Following a 1.5 hour incubation in SOC medium at 30° C., the cells were plated on 201 g/ml chloramphenicol LB plates (pre-dried and pre-warmed to 42° C.) and incubated at 42° C. for 24 to 48 h. The single colonies raised on the chloramphenicol plates were streaked on 5% (wt/vol) sucrose-containing LB agar and chloramphenicol-containing LB agar respectively, and the plates were incubated overnight at 37° C. Those colonies on sucrose plates and derived from sucrose-sensitive, chloramphenicol-resistant colonies were screened for chromosomal deletion of the appropriate efflux pump genes by PCR amplification of the efflux genes with primers originally used in construction of plasmids carrying deleted efflux pump genes. Using the approach as described above, ΔemrE mutants of K1537 (i.e., XZL986 #12 and #18) were obtained and confirmed. An ΔemrD mutant of XZL986 (i.e. XZL992) was also obtained.

The bacterial strains and plasmids used in this study are listed in Table 4.

TABLE 4

| Strain | Genotype |
|---|---|
| AG100MA | Wildtype |
| AG112MA | marR |
| XZLI035 | ΔwaaP |
| AG1OOA | ΔacrAB |
| XZL1033 | ΔacrAB ΔwaaP |
| AG10OAX | ΔacrAB ΔacrEF |
| XZL986 | ΔacrAB ΔacrEF ΔemrE64 |
| XZL992 | ΔacrAB ΔacrEF |
| | ΔemrE ΔemrD |
| XZL1O34 | ΔacrAB ΔacrEF ΔemrE |
| | ΔemrD ΔwaaP |

The wild-type strain AG100 and marR mutant AG112MA of E. coli were kindly provided by S. B. Levy of Tufts University, Boston, Mass. The acrAB mutant, AG100A, and the acrAB, acrEF mutant, AG100AX, derived from strain AG100 were kindly provided by H. Nikaido of University of California at Berkeley, Calif. The gene replacement vector, pK03, was a kind gift from G. M. Church of Harvard University, Boston, Mass. Luria-Bertani (LB) broth (1% [wt/vol] Difco™ tryptone, 0.5% [wt/vol] Difco™ yeast extract, and 0.5% [wt/vol] NaCl) and agar (LB broth containing 1.5% [wt/vol] agar) were used as the growth media throughout and bacterial cells were cultivated at 37° C. or 30° C. as specified below. In some instances, SOC broth (2% [wt/vol] Difco™ tryptone, 0.5% [wt/vol] Difco™ yeast extract, 10 mM NaCl, 2.5 mM KCl, 10 mM MgSO₄, 10 mM MgCl₂, and 20 mM glucose) was used. Plasmids were maintained in E. coli with appropriate antibiotic selection (pBluescript II S K[+], pBAD18 [Guzman et al., (1995), J Bacteriol, 177(4), 4121-4130] and pUC151A [Ma et al., (1993) J Bacteriol 175(19), 6299-6313], 100 μg of ampicillin per ml; pK03 [Link et al., 1997], 30 μg of chloramphenicol per ml; pRK415 and pRSP41, 10 μg of tetracycline per ml).

### Example 3

Construction of ΔemrE. ΔemrD Mutants in an ΔacrAB ΔacrEF Background

The method of Link et al (J. Bacteriol 0.1997) was used for construction of markerless emrE, emrD, deletion mutants of E. coli. Briefly, PCR was used to amplify sequences 5′ and 3′ to the region to be deleted in each gene, using chromosomal DNA of E. coli AG100AX as template. Primer pairs (2) for each gene deletion were designed using the E. coli genome sequence database (emrE from GenBank accession number AE000160; emrD from GenBank accession number AE000445).

Sequence 5′ to emrE was amplified using primers emre1xz (SEQ ID NO:48) (5′-AATTIGGATCCGCCAACACTGCGACAGCGT-3′; BamHI site bold; erme1xz: anneals to 1044-1023 bp upstream of the emrE start codon) and emre2xz (SEQ ID NO:47) (5′-ACTTGAATTCATTAAGGTTGTACCAATGAC-3′; EcoRI site bold; anneals 65-43 bp downstream of the emrE start codon) while sequence 3′ to this gene were amplified with primers enire3xz (SEQ ID NO:40) (5′-ACTGGAATTCACGAAGCACACCACATTAA-3′; EcoRI bold, anneals 21-1 bp upstream of the emrE stop codon) and emre4xz (SEQ ID NO:39) (5′-CTTGGTCGACAAGACGCCGTCATAATC-3, SalI site bold; anneals 777-758 bp downstream of the emrE stop codon). Similarly, sequence 5′ to emrD was amplified using primers emrd1xz (SEQ ID NO: 32) (5′-ATTAGGATCCACACGCACGACGACCACTG-3′; BamHI site bold; anneals 999-979 bp upstream of the emrD start codon) and emrd2xz (SEQ ID NO: 31) (5′-ACTTGAATTCTGACCGACGGCCACGAGT-3′; EcoRI site bold; anneals 80-61 bp downstream of the emrD start codon), while sequence 3′ to this gene was amplified with primers emrd3xz (SEQ ID NO:24) (5′CATGGAATTCGCATCAGGGGCAGCCCGTT-3′; EcoRI bold; anneals 24-5 bp upstream of the emrD stop codon) and emrd4xz (SEQ ID NO:23) (5′-GAAAGTCGACCTGCCCTACACCAACAGC-3′; SalI site bold; anneals 953-926 bp downstream of the emrD stop codon).

The PCR mixtures contained 50 ng of E. coli chromosomal DNA, 40 pmol of each primer, 0.2 mM (each) deoxynucleoside triphosphate, 2 mM MgSO₄ and 10% (vol/vol) dimethylsufoxide in 1× thermo reaction buffer (New England Biolabs, Mississauga, Ontario, Canada) and were heated for 5 min at 94° C. before the addition of 2 U of Vent DNA polymerase (New England Biolabs) per reaction. The reaction was then processed for 30 cycles of 40 sec: at 94° C., 40 sec at 56° C. and 40 sec at 72° C. before finishing with 10 min at 72° C. The PCR products to be used in each deletion were purified using a Qiaquick™ PCR purification kit (Qiagen Inc.), digested with BamHI and EcoRI or EcoRI and Sail as appropriate, and cloned into EcoRI-SalI-restricted pBluescript II SK(+) via a three-piece ligation. The resultant vectors, pXZL967 and pXZL983, carried deletions in emrE and emrD respectively, as confirmed by restriction analysis and nucleotide sequencing. Digestion of plasmids pXZL967 and pXZL983, with BamHI and SalI released fragments carrying the PCR-generated deletions of emrE (246 bp in-frame deletion), and emrD (1101 bp in-frame deletion), respectively, which were then cloned into BamHI-SalI-restricted pK03, yielding pXZL975, pXZL987, and pXZL1024 (selected on chloramphenicol (30 μg/ml)-containing LB agar at 30° C.). These pK03 derivatives were subsequently prepared using a Qiagen DNA midi-kit and electroporated into freshly-made electro-competent E. coli host cells (AG100 and its derivatives) at 2.5 kV, 25 μF and 200 ohms. Following a 1.5 h-incubation in SOC broth at 30° C., the cells were plated on chloramphenicol (20 or 30 μg/ml)-containing LB plates (prewarmed to 42° C.) and incubated at 42° C. for 24 to 48h. The resultant single colonies obtained were streaked onto LB agar containing 5% (wt/vol) sucrose, and sucrose-resistant colonies screened for chromosomal deletion of the appropriate efflux pump genes using PCR with primer pairs emre1xz/emre4xz (for emrE) and emrd1xz/emrd4xz (for emrD). Chromosomal deletions in emrE and emrD, were both obtained. For the construction of the construction of the ΔacrAB ΔacrEF ΔemrE ΔemrD quadruple pump mutant, the emrE and emrD deletions were introduced sequentially into E. coli strain AG100AX using the procedures outlined above.

Antimicrobial susceptibility assays indicated that ΔemrE mutants are more susceptible to ethidium bromide (32 fold increase in MIC values), but not to other antimicrobial agents, while other deletion mutants have lesser effects on ethidium sensitivity.

### Example 4

Reconstitution of acrAB Expression

Cloning of acrAB

An acrAB fragment (ca. 4.5 kb) was released from pRSP41 by digestion with SstI and SalI and blunt-ended with T4 DNA polymerase treatment. The blunt-ended acrAB fragment was then cloned into SmaI-digested, alkaline phosphatase-treated pBAD18. Transformants were selected on LB agar plates containing 0.2% (wt/vol) glucose and 100 μg/ml ampicillin. Restriction digestion and DNA sequencing confirmed the cloning of acrAB into pBAD 18, in the same (plasmid pXZL962) and opposite (Plasmid pXZL961) orientation with respect to the resident ara promoter of pBAD18. Complementation of the antibiotic susceptibility of an acrAB-deficient E. coli (strain AG100A) by plasmids pXZL961 and pXZL962 was determined with or without induction of the cloned genes with 0.1% (wt/vol) arabinose. Expression of acrAB was also analyzed by SDS-PAGE and Western immunoblotting with an anti-AcrA polyclonal antibody (kindly provided by H. Nikaido).

Functional Analysis of Cloned acrAB in Wild-Type and acrAB-Deficient E. coli Strains.

To determine effect of the cloned acrAB genes on antibiotic susceptibility, the plasmids pUC151A and pRSP41 containing cloned acrAB on pUC19 (i.e., pUC151A) and pRK415 (i.e., pRSP41), respectively, were used to transform E. coli DH5α (wild type) and K1537 (E. coli AG100 ΔacrAB ΔacrEj). Transformants were selected on LB agar plates containing 0.2% (wt/vol) glucose and 100 μg/ml ampicillin (for pUC19 based plasmids) or 10 μg/ml tetracycline (for pRK41 5-derived plasmids). Antibiotic susceptibility of E. coli strains harboring appropriate plasmids was determined. The results indicated that the cloned acrAB genes on PRSP41 did not significantly confer additional or elevated resistance upon wild-type cells but did restore antibiotic resistance to acrAB-deficient strains.

Cloning of acrAB into pBAD18.

To tightly control expression of acrAB genes, the acrAB genes were cloned into a well-regulated vector, pBAD18 (Guzman et al. 1995. J. Bacteriol 177:4121-4130). The acrAB fragment (ca. 4.5 kb) was released from pRSP41 by digestion with SstI and SalI and blunt-ended with T4 DNA polymerase treatment. The acrAB genes were then subcloned into SmaI-digested, alkaline phosphatase-treated pBluescript II SK(+) and pBAD 18, respectively. Transformants were selected on LB agar plates containing 0.2% (wt/vol) glucose and 1001 g/ml ampicillin. Restriction digestion and DNA sequencing confirmed the cloning of acrAB into pBAD 18 in the same (pXZL962) and opposite (pXZL961) orientation with respect to the ara promoter on pBAD18. Complementation of antibiotic susceptibility of the acrAB-deficient E. coli (K1537) by pXZL961 and pXZL962 was determined in the presence or absence of 0.1% (wt/vol) arabinose (induces expression of genes cloned into pBAD vectors). Expression of acrAB was analyzed by SDS-PAGE and Western immunoblotting with anti-acrA polyclonal antibody. Both pXZL961 and pXZL962 promoted expression of acrAB in strain K1537 and restored antibiotic resistance, independent of arabinose. This is likely due to the activity of the acrAB promoter. Western immunoblotting showed that acrA was produced in cells harboring either pXZL961 or pXZL962, with higher production in cells carrying pXZL962 in the presence of arabinose.

Since expression of acrAB was independent of the arabinose-inducible pBAD promoter, it is necessary to delete the acrAB promoter to permit tightly regulated, arabinose-inducible acrAB expression from pBAD vector pXZL962. An approach was then used to remove acrAB promoter by NheI and XhoI digestion. A Shine-Dalgarno sequence (a 14 bp NheI-XhoI fragment), resulting from annealing two oligonucleotides, was cloned into NheI-XhoI restricted pXZL962 (see below).

Construction of Plasmids Carrying Inducible acrAB Genes.

As described above, the pBAD 18-based plasmids carrying acrAB gene (i.e., pXZL961 and pXZL962) have acrAB expression independent of arabinose induction as a result of the presence of the native acrAB promoter upstream of acrAB. The acrAB promoter was removed by digestion of plasmid pXZL962 with NheI and XhoI and the digested plasmid was then ligated with an in-vitro annealed double-stranded Shine-Dalgamo sequence to yield plasmid pXZL991. acrAB expression in pXZL991 was inducible in the presence of arabinose as demonstrated by the plasmid's ability to restore antibiotic resistance of arcAB-deficient strains in the presence of arabinose.

The cloned acrAB genes on pRSP41, pXZL961, and pXZL962 were introduced into strain XZL986 and the effect of the acrAB genes on antimicrobial susceptibility of the strain was assessed using MIC determinations in LB broth at 37° C. MIC data showed that the cloned acrAB genes could restore the resistance phenotype of XZL986. The ethidium resistance level was still lower than that of wild type E. coli cells (MIC of ethidium bromide is generally 128 to 256 pg/ml), indicting that emrE does contribute the ethidium bromide resistance of wild-type E. coli strains. Still, consistent with previous data arabinose has little effect on pXZL961 or pXZL962 mediated multidrug resistance. Functional analysis of a reconstituted system Introduction of the cloned acrAB genes into the multiply pump-deficient strain of E. coli markedly enhanced antimicrobial resistance and energy-dependent exclusion of ethidium bromide from the cell (consistent with an ability to export these agents), but it did not appear to promote efflux of previously accumulated ethidium from the cell. To see if this was an artifact of the assay (possibly a multicopy vector would sequester ethidium bromide, a known DNA-binding agent, preventing its acquisition by the acrAB efflux system), we cloned the emrE ethidium efflux gene and then assessed its ability to promote ethidium efflux in E. coli. The cloned emrE pump did, in fact, export ethidium, suggesting that while it is able to export agents from the cytoplasm, AcrAB likely accesses substrates as they enter the cell (i.e. from the periplasmic side of the inner membrane) and is unable to access these once they have entered the cell. The most likely explanation is that AcrAB has a low affinity for ethidium bromide (while EmrE has a high affinity), and given the expected low concentration of available ethidium bromide inside E. coli (most will be bound to DNA) measurable export cannot be seen. In contrast, ethidium entering the cell (prior to its reaching the cytoplasm) will be at a much higher available concentration and, thus, efficiently captured and expelled by AcrAB.

### Example 5

High Throughput Ethidium Bromide Accumulation Assay

Growth of Strains

All strains are grown overnight on a suitable medium, Luria-Bertani (LB) broth is particularly preferred for many enteric species., and strains containing plasmids are grown in LB containing 100 μg/ml of ampicillin for β-lactamase encoding plasmids, or any other antibiotic suitable for maintaining the relevant plasmid. The next morning cells are diluted 1:10 into fresh LB broth and the cells are grown for 4 hours at 37° C. Cells are centrifuged at 1,000×g for 15 minutes at room temperature and then suspended in 10 ml of 0.05 M phosphate buffer, pH 7.0, containing 100 mM sodium chloride (Buffer-A). The optical density is measured at 600 nm and an aliquot of cells sufficient to make 50 ml of cells at an optical density of 0.5 at 600 nm is centrifuged. The cell pellet is suspended in 50 ml of buffer-A containing 50 mM formate. Cells are used in screening assays for up to 3 hours after suspension in buffer-A. Formate serves as an energy source to maintain the proton gradient across the cell membrane necessary for efflux pump activity.

The Assay

The preferred fluorescent substrate used to measure the activity of the acrAB efflux pump in E. coli strain K17864 (ΔacrAB, ΔacrEF, ΔemrE, ΔemrD, ΔwaaP, [acrAB]) is ethidium bromide. Ethidium bromide is an environment sensitive probe and has a 20 to 30-fold increase in fluorescence when bound to DNA. In the presence of functioning efflux pumps, E. coli cells are effective in preventing ethidium bromide from entering and accumulating in the cell. However, if the permeability of the cell membrane is compromised or the efflux pumps are inactivated, a significant increase in accumulation of ethidium bromide entering and accumulating in the cell can be observed as an increase in the binding of ethidium bromide to DNA and a corresponding increase in fluorescence. The accumulation and increase in fluorescence of ethidium bromide is used as a screen for efflux pump inhibitors. Libraries of compounds are plated into 384 well plates in a 1 μl volume from stocks made at 2.5 mM in DMSO. Wells reserved for measuring the activity of untreated or control cells receive only DMSO. A 10 μl aliquot of 100 μM ethidium bromide made in buffer-A containing 50 mM formate is added to each well, and the accumulation assay is started by the addition of 90 μl of cells. Cells are suspended in buffer-A containing 50 mM formate at an optical density of 0.5 as measured at 600 nm. The change in fluorescence is measured using an LJL fluorescent plate reader or a Zeiss fluorescent plate reader using an excitation wavelength of 530 nm and an emission wavelength of 600 nm and the intracellular concentration of the dye measured.

### Example 6

The “Plus-Minus Approach”

By judicious selection of the strains tested it is possible to determine the mechanism of action of any test compound. A compound which increases ethidium bromide accumulation in strain K1764 (ΔacrAB, ΔacrEF, ΔemrE, ΔemrD, ΔwaaP [acrAB], wherein acrAB expressed on a plasmid) but not in K1750 (ΔacrAB, ΔacrEF, ΔemrE, ΔemrD, ΔwaaP wherein acrAB is not expressed) is likely a specific AcrAB efflux pump inhibitor, while a compound which increases ethidium bromide accumulation in both strains is likely a membrane permeabilizer (either inner or outer membrane) or is operating by some other non-specific mechanism.

A compound which increases permeability in K1764 and K1747 (ΔacrAB, ΔacrEF, ΔemrE, ΔemrD) is likely a permeabilizer of both the inner and outer membranes.

The advantage of using strains which either do or do not express an AcrAB efflux pump (as described above) is that the data analysis, especially kinetic analysis is simplified in such a comparative system as opposed to a system where acrAB is simply expressed at different relative levels. As discussed below whenever strains either expressing or not expressing a particular pump are employed it is possible to isolate the apparent rate constants for transport in a fairly straightforward way.

### Example 7

Determination of Difference Yields of Apparent Rate Constants

The fundamental properties of the kinetics of accumulation or efflux via transporters are based on mathematical analyses of compartments. This is a topic generically within the mathematical heading of linear differential equations. In the particular cases applicable to influx and efflux, movement of compounds—whether antibiotics or markers of transport—occurs both via passive permeability and via active transport. Both processes must be accounted for in the analyses. Moreover, in Gram-negative bacteria two membrane barriers must be considered, the inner or plasma membrane and the outer membrane containing lipopolysaccharide (LPS). From the two-compartment system, i.e. the periplasmic space and the cytoplasm, and the membranes separating the spaces, equations may be derived describing the details of the kinetics of transport. Such equations are useful in developing the most sensitive method for designing a screen, in analyzing data derived from screening, as well as in characterization of compounds discovered using the screen.

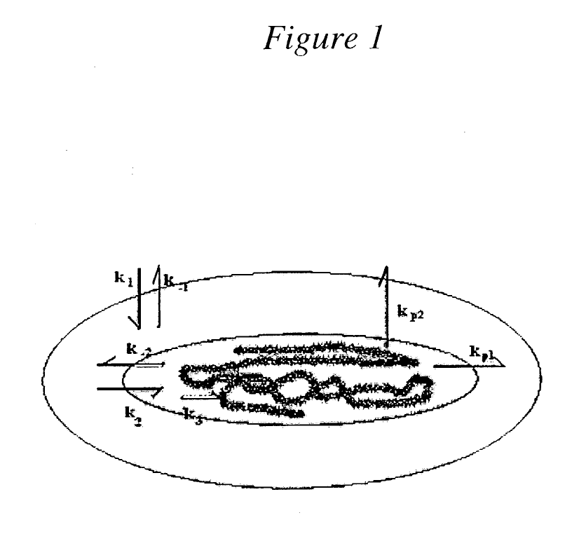

FIG. 1 outlines the important kinetic processes and compartments relevant to the underlying mathematical analyses of data from efflux transport. (Note: the figure is not in accurate proportions.)

In the model, passive rate constants are given number designations. The rate constants k₁ _{and k}₁ are the rate constants for passive permeability into and out of the periplasmic space, respectively. The rate constants k₂ and k₂ are associated with passive permeability into and out of the cytoplasm, respectively. The k₃ rate constant is for the association of a dye, such as ethidium bromide, with DNA in the cytoplasm. Association with DNA for ethidium bromide is considered as one-way for the time scale of the experimental conditions usually used for testing efflux transporters. However, equivalent equations may also be readily developed, if binding of the dye is considered reversible and a k₋₃ term for the dissociation of dye from DNA is included. DNA is represented by the thick, irregular line inside the cytoplasm. The rate constants for two types of efflux transporters are represented by kₚ₁ and kₚ₂. The process with the kₚ₁ rate constant is associated with an efflux pump that pumps substrate from the cytoplasm into the periplasmic space, consistent with emr-type transporters. The process with kₚ₂ as its rate constant is for an efflux pump that is pumping substrate from the cytoplasm directly into the extracellular space, which is consistent with an AcrAB-TolC complex. For brevity and ease of manipulation in the mathematical description, the symbol A is used to represent the concentration of an antibiotic or marker substrate, such as ethidium bromide, in the extracellular space, B is used for the concentration in the periplasmic space, and C is used for the concentration in the cytoplasmic space. Where needed, D is the concentration of ethidium bromide in associated with DNA in the cytoplasm. The concentration of marker or antibiotic is assumed to be constant for the experimental protocol so that A=A₀. The assumption was tested explicitly in experiments that define the limits of the assumption. The differential equations describing these processes are:

$$\begin{matrix}\frac{ⅆ B}{ⅆ t} = {k}_{1} {A}_{0} - ( {k}_{2} + {k}_{- 1} ) B + ( {k}_{- 2} + {k}_{p1} ) C & ( 1 a ) \\ \frac{ⅆ C}{ⅆ t} = {k}_{2} B - ( {k}_{- 2} + {k}_{p1} + {k}_{p2} - {k}_{3} ) C & ( 1 b ) \\ \frac{ⅆ C}{ⅆ T} = {k}_{3} C & ( 1 c )\end{matrix}$$

This is a series of first-order linear differential equations, which can be solved for B and C using standard mathematical techniques. The general form of the solutions is:
C=Q₁e^{r}₁^{t}+Q₂e^{r}₂^{t}+G₁ₖ (2a)
B=Q₃e^{r}₁^{t}+Q₄e^{r}₂^{t}+G₂ₖ (2b)
D=Q₁^{k}₃e^{r}₁^{t}+Q₂^{k}₃e^{r}₂^{t}+^{k}₃^{tG}₁ₖ (2c)

The terms Q₁, Q₂, Q₃, Q₄, r₁, r₂, G₁ₖ, and G₂ₖ are combinations of the microscopic rate constants, and t is time. The coefficients and exponents are generically termed “macroscopic coefficients” and “macroscopic rate constants”, since they are compilations of the fundamental or microscopic rate constants underlying the details of the transport processes. In cases where only a dye associated with intracellular DNA is measured, as with ethidium bromide, the result is expressed and quantified using the solution to D, only. The equations have the same mathematical form for dyes that are environment sensitive and accumulate in membranes, such as TMA-DPH, although the meanings of some of the macroscopic terms are somewhat different. In the latter case, the concentration of dye in the outer membrane is represented by B and concentration of accumulated dye in the inner or plasma membrane is represented by C. Accumulation of the highly fluorescent form of the dye occurs in both the outer and inner membranes, so what is observed is the sum of B+C. Meanings and interpretation of the individual terms in the equations are complex for the coefficients, specifically Q₁ through Q₄ and the combination of terms that precede the exponentials, e. However, there is a combination of the exponents, r₁ and r₂, that is particularly informative. In the solution of the differential equations r₁ and r₂ are each the solution of the same quadratic equation, which may be represented in common algebraic form as

$$x = \frac{- b \pm \sqrt{{b}^{2} - 4 ac}}{2 a}$$

For the present argument, each of the letter coefficients—a, b and c—is a combination of the microscopic rate constants. For the particular equations useful for efflux transport, a=1 which simplifies the denominator. More importantly, while the individual exponents, r₁ and r₂, are relatively complicated and not very informative because they are complementary solutions to the quadratic, the sum of r₁ and r₂ yields a simple combination of the rate constants. For example, for the system outlined in the diagram the sum of r₁ and r₂=(k₋₂+k₂+k₋₁+kₚ₁+kₚ₂). For time course data generated using one of the environment-sensitive fluorescent probes, the data are fitted to the general form of the appropriate equation. That is, the appropriate equation to use is the solution of B+C for membrane dyes and the solution for D when DNA binding dyes are used. The equation is fitted to the data using non-linear least squares regression analysis, which yields values for the macroscopic constants—Q₁ through Q₄, r₁ and r₂ and values for the Gₖ coefficients. As described above, the sum of r₁ and r₂ yields a sum of most of the microscopic rate constants, including kₚ₁ and kₚ₂, which are the relevant constants for quantifying efflux pumps. A unique and particularly useful feature of the analysis described here is the use of different strains of bacteria with the probes of efflux activity. Analyzed time course data using one or more of the efflux transport probes generated using one or more of the strains containing various efflux pump and efflux pump mutants will yield different values for the respective r₁+r₂ sums. For example, rate constants for a strain containing both forms of the efflux pump, i.e. those that pump substrate from inside the cell to the extracellular space and pumps that pump from inside the cytoplasm to the periplasmic space, may be compared with mutated cells of the same strain where the pump activity has been eliminated. The values for r₁+r₂ in the strain with both types of pump yield (k₋₂+k₂+k₋₁+kₚ₁+kₚ₂), and r₁+r₂ for the strain with no efflux pumps yields (k₋₂+k₂+k₋₁). Simple subtraction of the two values produces a measure attributable solely to efflux pump activity. Similarly, strains with only one type of efflux pump may be compared with strains that have no efflux pumps to yield the rate constants associated with only one type of efflux pump. More examples of the procedure used to determine rate constants for particular efflux pumps using various strains and mutants is described in Table 3.

TABLE 3
Experiments to Determine Kinetic Properties of Inhibitors

| Experiment type | Sum of apparent rate | |
|---|---|---|
| and Strains Used | constant yields | Difference Yields |
| Uncoupler-treated | k₋₁ + k₂ + k₋₂ | |
| or Δ Quad | | |
| Wild-type | k₋₁ + k₂ + k₋₂ + | Type II − Type I |
| | kₚ₁ + kₚ₂ | kₚ₁ + kₚ₂ |
| AcrAB expressing | k₋₁ + k₂ + k₋₂ + | Type III − Type I |
| | kₚ₂ | kₚ₁ |
| Emr E or D | k₋₁ + k₂ + k₋₂ + | Type IV − Type I |
| expressing | kₚ₁ | kₚ₂ |

Results from appropriate choices of mutated and non-mutated strains may be combined with appropriate choices of efflux pump substrates and experimental conditions. From the combined results, dose-response relationships may be quantified. These are useful both in assessing potency of efflux pump inhibitors but also for determining the mechanism of action of inhibitors, e.g. competitive versus non-competitive mechanisms. An example of the state of the art for analysis of efflux transport kinetics prior to our derivation was recently published in the Journal of Biological Chemistry by Walinsley, et al. (2001. Vol. 276, pp. 6378-6391). Although the authors developed some of the requisite mathematical models of efflux behavior, analysis and understanding of macroscopic rate constants was not described. Moreover, parameter values for the macroscopic constants were determined by an empirical approach to the analysis. Specifically, the fit of an equation to the data was dictated solely by increasing the number of exponential terms until the model converged on the data. Such an approach provides a very limited ability to assign a biochemical mechanism or to localize an inhibitory mechanism to the empirical coefficients in the equations. An important feature missing from such an analysis is the ability to distinguish effects on passive permeability from effects directly on transport proteins. In addition, the published approach made no use of the additional functional information provided by assessing a variety of strains with various pump deletions and additions. This demonstrates the increased utility of the approach presented in the present work.

### Example 8

Examples of Direct Assays of Membrane Integrity

Beta-lactamase assay: β-lactamases are periplasmic enzymes that hydrolyse β-lactam antibiotics. An increase in β-lactamase activity following treatment with compounds is used as an assay to screen for compounds causing a change in outer membrane permeability. The β-lactamase assay is used to distinguish between compounds increasing ethidium bromide accumulation as a result of increased outer membrane permeability from compounds causing an increase in ethidium bromide accumulation as the result of efflux pump inhibition. An example of a strain of E. coli used to measure β-lactamase activity is strain ML-35 (pBR322), a constitutive β-galactosidase producing, LacY permease deficient strain (i⁻, y⁻, z⁺ with β-lactamase expressed on a plasmid). Cells are grown overnight at 37° C. in LB broth containing 100 μg/ml ampicillin, and the next morning a 0.5 ml aliquot is diluted into 30 ml of fresh LB without ampicillin and grown an additional 3 hours at 37° C. Cells are centrifuged at 2,500×g for 10 minutes at room temperature and the cell pellet is suspended in 20 ml of 50 mM sodium phosphate buffer pH 7.4. Cells are again centrifuged and the cell pellet adjusted to an optical density of 0.5 at 600 nm in phosphate buffer pH 7.4. Cells are used immediately to assay β-lactamase activity. The β-lactamase assay is run in a 384-well plate in a final volume of 50 μl per well. To each well of the 384-well plate, 1 μl of test compound is added from stocks made at 2.5 mM in DMSO followed by 9.0 μl of 50 mM phosphate buffer, pH 7.4. Untreated cells used as controls receive DMSO only. Next, 20 μl of the β-lactamase substrate, nitrocefin, is added from a stock made at 400 μM in phosphate buffer. The assay is started by the addition of 20 μl of cells. The kinetics of the reaction are monitored using a Spectromax plate reader set at a wavelength of 482 nm. Readings are taken every 40 seconds for 30 minutes.

β-galactosidase assay: β-galactosidase is an intracellular enzyme, and an increase in β-galactosidase activity following treatment with compounds is used to screen for compounds causing a change in inner membrane permeability. An example of an E. coli strain used for determination of intracellular β-galactosidase activity and growth and preparation of cells is ML-35, identical to that described above for the β-galactosidase assay. The assay protocol for β-galactosidase is also the same as that described for β-lactamase with the exception that the substrate used is o-nitrophenyl-beta-galactoside (ONPG). ONPG is used from a stock solution made at 5.2 mM in phosphate buffer. The wavelength used to monitor galactosidase activity is 420 nm.

Other methods of determining whether a compound affects the intrinsic membrane integrity of bacterial cells are well known in the art and are encompassed by the invention. Another method of determining whether a test agent is directly acting at the AcrAB efflux pump is to assess direct efflux of membrane bound substrates out of the cell (“the active efflux method”). This method is useful because it largely eliminates any confounding influences of concurrent passive accumulation of marker substrate compound within the cell and because it distinguishes efflux from gross membrane disruption.

In one embodiment, the invention contemplates preloading AcrAB expressing bacterial cells with AcrAB substrate in the presence of a proton gradient disrupting compound. Treatment with the proton gradient disrupting compound destroys the proton gradient required by the AcrAB transporter and, therefore, the AcrAB substrate accumulates in the cells. The passive leakage of AcrAB substrate out of the cell is measured by diluting preloaded cells into buffer and observing the decrease in substrate concentration within the cells and a concomitant increase in the external medium. An acidic energy source reestablishes the proton gradient and active efflux of AcrAB substrate from cells is measured following the addition of the acidic energy source. We have found that the active efflux assay is particularly preferred for marker substrate dyes which are membrane bound lipophilic dyes but less well suited for nucleic acid binding dyes such as ethidium bromide.

### Example 9

Assays to Measure Direct Efflux

Direct Efflux of TMA-DPH

Compounds identified as efflux pump inhibitors can be further evaluated by measuring their effects on the direct efflux of TMA-DPH. Stationary phase or log phase E. coli were harvested by centrifuging the cells at 2,000×g for 10 minutes in a Beckman table top centrifuge. Cells were resuspended in 0.5 M phosphate buffer, pH 7.0, containing 100 mM sodium chloride to a density of 10 O.D. measured at 600 nm. Cells were then treated with 1 μM TMA-DPH and 20 μM carbonylcyanide m-chlorophenyl-1,3,5-hexatriene (CCCP) for 15 minutes at room temperature. CCCP disrupts the proton motive force required by the acrAB efflux transporter for activity. In the presence of CCCP the efflux pump is inhibited and TMA-DPH accumulates. Cells are then centrifuged and resuspended in buffer at a density of 10 O.D. at 600 nm, and 50 μl of cells are diluted into a cuvette containing 1.95 ml of buffer containing 50 mM formate with and without potential inhibitors. The rate of efflux of TMA-DPH in cells is measured by monitoring the decrease in fluorescence with time at an excitation wavelength of 360 nm and an emission wavelength of 425 nm.

### Example 10

Brief Description of the Sequence Listing

TABLE 5

| SEQ ID NO: | SEQUENCE DESCRIPTION | GENBANK DESCRIPTION # |
|---|---|---|
| SEQ ID NO: 1 | Escherichia coli (K12 strain) acrA DNA coding sequence | U00734 |
| SEQ ID NO: 2 | Escherichia coli AcrA protein sequence | AAA67134 |
| SEQ ID NO: 3 | Klebsiella pneumoniae acrA DNA coding sequence | Derived from AJ318073 |
| SEQ ID NO: 4 | Klebsiella pneumoniae AcrA protein sequence | CAC41008 |
| SEQ ID NO: 5 | Pseudomonas aeruginosa mexA DNA coding sequence | Derived from L11616 |
| SEQ ID NO: 6 | Pseudomonas aeruginosa MexA protein sequence | AAA74436 |
| SEQ ID NO: 7 | Salmonella enterica acrA DNA coding sequence | Derived from GenBank AL627267 |
| | | (Salmonella enterica serovar typhi) |
| | | substantially similar to AE008717 |
| | | (Salmonella enterica serovar typhimurium) |
| SEQ ID NO: 8 | Salmonella enterica AcrA protein sequence | CAD04961 |
| SEQ ID NO: 9 | Enterobacter aerogenes acrA DNA coding sequence | Derived from GenBank AJ306389 |
| SEQ ID NO: 10 | Enterobacter aerogenes AcrA protein sequence | CAC35724 |
| SEQ ID NO: 11 | Escherichia coli acrB DNA coding sequence | Derived from GenBank #U00734 |
| SEQ ID NO: 12 | Escherichia coli AcrB protein sequence (K12 strain) | AAA67135 |
| SEQ ID NO: 13 | Klebsiella pneumoniae acrB DNA coding sequence | Derived from GenBank #AJ318073 |
| SEQ ID NO: 14 | Klebsiella pneumoniae AcrB protein sequence | CAC41009 |
| SEQ ID NO: 15 | Pseudomonas aeruginosa mexB DNA coding sequence | Derived from GenBank #L116176 |
| SEQ ID NO: 16 | Pseudomonas aeruginosa MexB protein sequence | AAA74437 |
| SEQ ID NO: 17 | Salmonella enterica acrB DNA coding sequence | Derived from GenBank AL627267 |
| | | (Salmonella enterica serovar typhi) |
| | | substantially similar to AE008717 |
| | | (Salmonella enterica serovar typhimurium) |
| SEQ ID NO: 18 | Salmonella typhimurium AcrB protein sequence | CAD04960 |
| SEQ ID NO: 19 | Enterobacter aerogenes acrB DNA coding sequence | Derived from GenBank #AJ366389 |
| SEQ ID NO: 20 | Enterobacter aerogenes AcrB protein sequence | CAC35725 |
| SEQ ID NO: 21 | Escherichia coli acrE DNA coding sequence | Derived from GenBank #M968487 |
| SEQ ID NO: 22 | Escherichia coli AcrE protein sequence | AAA02931 |
| SEQ ID NO: 23 | Primer sequence | N/A |
| SEQ ID NO: 24 | Primer sequence | N/A |
| SEQ ID NO: 25 | Pseudomonas aeruginosa mexC DNA coding sequence | Derived from GenBank #U57969 |
| SEQ ID NO: 26 | Pseudomonas aeruginosa MexC protein sequence | AAB41956 |
| SEQ ID NO: 27 | Salmonella enterica acrE DNA coding sequence | Derived from GenBank AE008856 |
| | | (Salmonella enterica serovar typhimurium) |
| | | substantially similar to partial |
| | | sequence GenBank #AL627278 |
| | | (Salmonella enterica serovar typhi) |
| SEQ ID NO: 28 | Salmonella typhimurium AcrE protein sequence | AAL22259 |
| SEQ ID NO: 29 | Escherichia coli acrF DNA coding sequence | Derived from GenBank #M96848 |
| SEQ ID NO: 30 | Escherichia coli AcrF protein sequence | AAA02931 |
| SEQ ID NO: 31 | Primer sequence | N/A |
| SEQ ID NO: 32 | Primer sequence | N/A |
| SEQ ID NO: 33 | Pseudomonas aeruginosa mexD DNA coding sequence | Derived from GenBank #U57969 |
| SEQ ID NO: 34 | Pseudomonas aeruginosa MexD protein sequence | AAB41957 |
| SEQ ID NO: 35 | Salmonella enterica acrF DNA coding sequence | Derived from GenBank AE008856 |
| | | (Salmonella enterica serovar typhimurium) |
| | | substantially similar to AL627278 |
| | | (Salmonella enterica serovar typhi) |
| SEQ ID NO: 36 | Salmonella enterica AcrF protein sequence | AAL22260 |
| SEQ ID NO: 37 | Escherichia coli emrE DNA coding sequence | Derived from GenBank #Z118877 |
| SEQ ID NO: 38 | Escherichia coli EmrE protein sequence | Derived from GenBank #CAA77936 |
| SEQ ID NO: 39 | Primer Sequence | N/A |
| SEQ ID NO: 40 | Primer Sequence | N/A |
| SEQ ID NO: 41 | Pseudomonas aeruginosa emrE DNA coding sequence | Derived from GenBank #AE004912 |
| SEQ ID NO: 42 | Pseudomonas aeruginosa EmrE protein sequence | AAG08375 |
| SEQ ID NO: 43 | Salmonella enterica emrE DNA coding sequence | Derived from GenBank AL627270 |
| | | (Salmonella enterica serovar typhi) |
| | | substantially similar to AE008773 |
| | | (Salmonella enterica serovar typhimurium) |
| SEQ ID NO: 44 | Salmonella enterica EmrE protein sequence | AAL20571 |
| SEQ ID NO: 45 | Escherichia coli emrD DNA coding sequence | (Derived from GenBank #L 10328 |
| SEQ ID NO: 46 | Escherichia coli EmrD protein sequence | P31442 |
| SEQ ID NO: 47 | Primer Sequence | N/A |
| SEQ ID NO: 48 | Primer Sequence | N/A |
| SEQ ID NO: 49 | Pseudomonas aeruginosa emrD DNA coding sequence | Derived from GenBank #AE004778 |
| SEQ ID NO: 50 | Pseudomonas aeruginosa EmrD protein sequence | AAG0961 |
| SEQ ID NO: 51 | Salmonella enterica emrD DNA coding sequence | Derived from GenBank AL627280 |
| | | (Salmonella enterica serovar typhi) |
| | | substantially similar to AE008877 |
| | | (Salmonella enterica serovar typhimurium) |
| SEQ ID NO: 52 | Salmonella enterica EmrD protein sequence | CADO3195 |
| SEQ ID NO: 53 | Escherichia coli waaP DNA coding sequence | Derived from GenBank #U00039 |
| SEQ ID NO: 54 | Escherichia coli WaaP protein sequence | AAC76654 |
| SEQ ID NO: 55 | Pseudomonas aeruginosa waaP DNA coding sequence | Derived from GenBank #AE004913 |
| SEQ ID NO: 56 | Pseudomonas aeruginosa WaaP protein sequence | AAG08394 |
| SEQ ID NO: 57 | Salmonella enterica waaP DNA coding sequence | Derived from GenBank AL627280 |
| | | (Salmonella enterica serovar typhi) |
| | | substantially similar to AE008873 |
| | | (Salmonella enterica serovar typhimurium) |
| SEQ ID NO: 58 | Salmonella enterica WaaP protein sequence | CAD03272 |
| SEQ ID NO: 59 | Primer Sequence | N/A |
| SEQ ID NO: 60 | Primer Sequence | N/A |
| SEQ ID NO: 61 | Primer Sequence | N/A |
| SEQ ID NO: 62 | Primer Sequence | N/A |
| SEQ ID NO: 63 | Primer Sequence | N/A |
| SEQ ID NO: 64 | Haemophilus influenza acrA DNA coding sequence | GenbankU32771 |
| SEQ ID NO: 65 | Haemophilus influenza AcrA protein sequence | Genbank AAC2255 Hypothetical protein HI0894 |
| SEQ ID NO: 66 | Haemophilus influenza acrB DNA coding sequence | Genbank U32771 |
| SEQ ID NO: 67 | Haemophilus influenza AcrB protein sequence | Genbank AAC22555 |

It will be clear that the invention may be practiced otherwise than as particularly described in the foregoing description and examples. Numerous modifications and variations of the present invention are possible in light of the above teachings and, therefore are within the scope of the invention. All publications and GenBank accessions cited herein are hereby incorporated by reference in their entirety.

1. A method for screening for an inhibitor of the AcrAB efflux pump comprising:
contacting an outer membrane permeabilized, gram negative bacterium which expresses an AcrAB efflux pump with an AcrAB marker substrate compound and a test agent, wherein at least one other transporter of said marker substrate compound lacks activity;
determining the intracellular concentration or rate of accumulation of said marker substrate compound at one or more times; and
comparing the intracellular concentration or rate of intracellular accumulation of said marker substrate compound at corresponding time in the presence of the test agent with the intracellular concentration or rate of intracellular accumulation in the presence of a positive or negative control, wherein an increase compared to the negative control or a similar concentration or accumulation compared to the positive control indicates that said test agent is an inhibitor of the AcrAB efflux pump.

2. The method of claim 1 wherein said acrAB marker substrate compound is fluorescent.

3. The method of claim 1 wherein said acrAB marker substrate compound is radioactive.

4. The method of claim 1 wherein said acrAB marker substrate compound is selected from the group consisting of ethidium bromide, acridine orange and proflavin.

5. The method of claim 1 wherein said gram negative bacterium has a disrupted waaP locus.

6. The method of claim 1 wherein said gram negative bacterium has a disrupted emrD locus.

7. The method of claim 1 wherein said gram negative bacterium has a disrupted emrE locus.

8. The method of claim 1 wherein said gram negative bacterium has a disrupted acrEF locus.

9. The method of claim 1 wherein said gram negative bacterium expresses the acrAB efflux pump from a single copy.

10. The method of claim 1 wherein said gram negative bacterium expresses the acrAB efflux pump from a multicopy plasmid.

11. The method of claim 1 wherein said gram negative bacterium has at least two disrupted loci selected from the group consisting of waaP, emrD, emrE, and acrEF.

12. The method of claim 1 wherein said gram negative bacterium is disrupted in each of the waaP, emrD, emrE, and acrEF loci.

13. The method of claim 9 wherein said gram negative bacterium has the ΔacrEF, ΔemrD, ΔwaaP, ΔemrE genotype.

14. The method of claim 10 wherein said gram negative bacterium has the ΔacrAB, ΔacrEF, ΔemrD, ΔwaaP, ΔemrE genotype.

15. The method of claim 1 wherein the method further comprises assessing cell permeability.

16. The method of claim 15 wherein cell permeability is assessed by a beta-lactamase assay.

17. The method of claim 15 wherein cell permeability is assessed by a beta-galactosidase assay.

18. The method of claim 1 wherein said bacterium is contacted with said marker substrate compound and said test agent in a first reaction vessel and said bacterium is contacted with said marker substrate compound in the absence of said test agent in a second reaction vessel.

19. The method of claim 1 wherein the method is performed within a multiwell plate.

20. The method of claim 1 wherein said contacting step is continued until the inward and outward fluxes of marker substrate compound reach a steady state.

21. The method of claim 20 wherein the determining step is performed once after steady state is attained.

22. The method of claim 1 wherein the method further comprises performing in parallel or sequentially the steps:
contacting an AcrAB marker substrate compound with an outer membrane permeabilized, gram negative bacterium that expresses an AcrAB efflux pump at reduced levels relative to the outer membrane permeabilized, gram negative bacterium of claim 1 with a test agent;
determining the intracellular concentration or rate of accumulation of said marker substrate compound at one or more times; and
comparing the intracellular concentration or rate of intracellular accumulation of said marker substrate compound at corresponding times in the presence of the test agent and selecting a test agent which increases the intracellular concentration of marker substrate compound in the outer membrane permeabilized gram negative bacterium of claim 1.

23. The method of claim 22 wherein the gram negative bacterium of claim 1 and the gram negative bacterium that expresses an AcrAB efflux pump at reduced levels are isogenic but for the expression of acrAB.

24. The method of claim 22 wherein the method further comprises determination of the difference yields of the sum of the apparent rate constants for the transport of said marker substrate compound.

25. The method of claim 1 wherein said method comprises performing in parallel or sequentially the following additional steps:
(a) adding a membrane bound AcrAB marker substrate and an uncoupling compound to a gram negative bacterium expressing the AcrAB efflux pump;
(b) removing said gram negative bacterium from the presence of the marker substrate compound and uncoupling compound; and
(c) contacting said bacterium with a proton donor, wherein said proton donor reestablishes a proton gradient;
(d) determining the transport activity of the AcrAB pump in the presence and absence of a test agent; and
(e) comparing the transport activity determined in the presence of the test agent to the transport activity determined in the absence of the test agent, wherein a decrease in transport activity indicates that said test agent is an AcrAB efflux pump inhibitor.

26. A recombinant gram negative bacterium expressing an AcrAB pump and comprising at least two disrupted loci selected from the group consisting of waaP, emrD, emrE and acrEF.

27. The recombinant gram negative bacterium of claim 26 which is disrupted in each of the waaP, emrD, emrE, and acrEF loci.

28. The recombinant gram negative bacterium of claim 26 comprising the ΔacrAB, ΔacrEF, ΔemrD, ΔwaaP, Δemr genotype E and wherein said AcrAB pump is expressed from a plasmid.

29. A recombinant gram negative bacterium of claim 28 wherein the plasmid is a multicopy plasmid.

30. A recombinant gram negative bacterium according to claim 26 comprising an AcrAB pump wherein the AcrAB pump is an Escherichia coli, Salmonella typhimurium, Pseudomonas aeruginosa, Enterobacter aergogenes, Klebsiella pneumoniae, or Haemophilus influenza gene product.

31. The method of claim 1, wherein at least two other transporters of said marker substrate compound lack activity.

32. The method of claim 1, wherein all other transporters of said marker substrate compound lack activity.

33. A method for screening for an inhibitor of the AcrAB efflux pump comprising:
contacting an outer membrane permeabilized, gram negative bacterium which expresses an AcrAB efflux pump and comprises a disrupted locus from one or more of waaP, emrD, emrE, or acrEF, with an AcrAB marker substrate compound and a test agent;
determining the intracellular concentration or rate of accumulation of said marker substrate compound at one or more times; and
comparing the intracellular concentration or rate of intracellular accumulation of said marker substrate compound at corresponding time in the presence of the test agent with the intracellular concentration or rate of intracellular accumulation in the presence of a positive or negative control, wherein an increase compared to the negative control or a similar concentration or accumulation compared to the positive control indicates that said test agent is an inhibitor of the AcrAB efflux pump.

34. A method for screening for an inhibitor of the AcrAB efflux pump comprising: contacting an outer membrane permeabilized, gram negative bacterium which expresses an AcrAB efflux pump, with an AcrAB fluorescent marker substrate compound and a test agent, wherein at least one other transporter of said marker substrate compound lacks activity;
determining the intracellular concentration or rate of accumulation of said marker substrate compound at one or more times; and
comparing the intracellular concentration or rate of intracellular accumulation of said marker substrate compound at corresponding time in the presence of the test agent with the intracellular concentration or rate of intracellular accumulation in the presence of a positive or negative control, wherein an increase compared to the negative control or a similar concentration or accumulation compared to the positive control indicates that said test agent is an inhibitor of the AcrAB efflux pump.

35. The method of claim 1, wherein said bacterium is recombinant.

36. The method of claim 33, wherein said bacterium is recombinant.

37. The method of claim 35, wherein said bacterium is recombinant.

38. The method of claim 33, wherein said marker substrate is fluorescent.


SEQUENCE LISTING (67 sequences)

SEQ ID NO: 1 - DNA, 1191 bp, Escherichia coli
atgaacaaaaacagagggtttacgcctctggcggtcgttctgatgctctcaggcagcttagccctaacaggatgtgacgacaaacaggcccaacaaggtggccagcagatgcccgccgttggcgtagtaacagtcaaaactgaacctctgcagatcacaaccgagcttccgggtcgcaccagtgcctaccggatcgcagaagttcgtcctcaagttagcgggattatcctgaagcgtaatttcaaagaaggtagcgacatcgaagcaggtgtctctctctatcagattgatcctgcgacctatcaggcgacatacgacagtgcgaaaggtgatctggcgaaagcccaggctgcagccaatatcgcgcaattgacggtgaatcgttatcagaaactgctcggtactcagtacatcagtaagcaagagtacgatcaggctctggctgatgcgcaacaggcgaatgctgcggtaactgcggcgaaagctgccgttgaaactgcgcggatcaatctggcttacaccaaagtcacctctccgattagcggtcgcattggtaagtcgaacgtgacggaaggcgcattggtacagaacggtcaggcgactgcgctggcaaccgtgcagcaacttgatccgatctacgttgatgtgacccagtccagcaacgacttcctgcgcctgaaacaggaactggcgaatggcacgctgaaacaagagaacggcaaagccaaagtgtcactgatcaccagtgacggcattaagttcccgcaggacggtacgctggaattctctgacgttaccgttgatcagaccactgggtctatcaccctacgcgctatcttcccgaacccggatcacactctgctgccgggtatgttcgtgcgcgcacgtctggaagaagggcttaatccaaacgctattttagtcccgcaacagggcgtaacccgtacgccgcgtggcgatgccaccgtactggtagttggcgcggatgacaaagtggaaacccgtccgatcgttgcaagccaggctattggcgataagtggctggtgacagaaggtctgaaagcaggcgatcgcgtagtaataagtgggctgcagaaagtgcgtcctggtgtccaggtaaaagcacaagaagttaccgctgataataaccagcaagccgcaagcggtgctcagcctgaacagtccaagtct

SEQ ID NO: 2 - protein, 397 aa, Escherichia coli
MNKNRGFTPLAVVLMLSGSLALTGCDDKQAQQGGQQMPAVGVVTVKTEPLQITTELPGRTSAYRIAEVRPQVSGIILKRNFKEGSDIEAGVSLYQIDPATYQATYDSAKGDLAKAQAAANIAQLTVNRYQKLLGTQYISKQEYDQALADAQQANAAVTAAKAAVETARINLAYTKVTSPISGRIGKSNVTEGALVQNGQATALATVQQLDPIYVDVTQSSNDFLRLKQELANGTLKQENGKAKVSLITSDGIKFPQDGTLEFSDVTVDQTTGSITLRAIFPNPDHTLLPGMFVRARLEEGLNPNAILVPQQGVTRTPRGDATVLVVGADDKVETRPIVASQAIGDKWLVTEGLKAGDRVVISGLQKVRPGVQVKAQEVTADNNQQAASGAQPEQSKS

SEQ ID NO: 3 - DNA, 1197 bp, Klebsiella pneumoniae
atgaacaaaaacagagggttaacgcctctggcggtcgttctgatgctctcaggcagcttagcgctaacaggatgtgacgataaaccggctcaacagggagcccagcacatgccggaagtcggtattgtgacgctcaaatccgcacctctacaaataaccaccgaactgccaggccgcaccagcgcctatcgcattgcggaagtccgtcctcaggtcagtggcattattttaaaacgtaacttcgtggaaggtagcgatatccaggccggcgtcttcctgtatcagatcgatccagccacctatcaaggcagctatgacagcgccaaaggcgacctggcaaaagcccaggcggcggcaaacatggatcaactgacggtcaagcgttatcagaaactgttgggcacccaatatattagtcaacaagactacgatcccgccgttgcgacggcgcaacaaaacaatgccgccgtggtcgcggcgaaaactgccgttgaaaccgcgcgcatcaatttggcctacacccaaaagtcacctctccgatcagcggccggatcgggtaaatcccccgtgaccgaaggggcgttggtacagaatggtcaaacgaccgccttggcaaccgttcagcaagtggatccgatctatgttgacgtcacccagtcgagcaatgatttcctgcgcctgaagcaggagctagccgacgcccgcctgaaacaggaaaacggcaaagcgaaagtggagctggtgactaatgacgggcttaagtatccgcagtccggcacgctggaattctcggatgtcaccgtcgatcagaccaccggctcaatcacgctacgcgctattttcccgaacccggatcacaccctgcttccggggatgttcgtccgtgcccgtctggaagaagggattaaccctgacgccctgctggtaccgcaacagggtgttacccgtacgccgcgcggcgacgccagcgtcatggtagtgggtgaaggcgataaagtcgaagtccgccaggtcactgcttctcaggcgatcggcgataaatggctggtcactgacggtctgaaatccggcgatcgcgttatcgtcaccggcctgcaaaaaatcaaaccaggtgtgcaggtaaaagcgcaggaagtagcttctgatgataaacagcaagccgcaggcaacgcgccatcagaacaaaccaagtcttaa

SEQ ID NO: 4 - protein, 398 aa, Klebsiella pneumoniae
MNKNRGLTPLAVVLMLSGSLALTGCDDKPAQQGAQHMPEVGIVTLKSAPLQITTELPGRTSAYRIAEVRPQVSGIILKRNFVEGSDIQAGVFLYQIDPATYQGSYDSAKGDLAKAQAAANMDQLTVKRYQKLLGTQYISQQDYDPAVATAQQNNAAVVAAKTAVETARINLAYTQKSPLRSAAGSGKSPVTEGALVQNGQTTALATVQQVDPIYVDVTQSSNDFLRLKQELADARLKQENGKAKVELVTNDGLKYPQSGTLEFSDVTVDQTTGSITLRAIFPNPDHTLLPGMFVRARLEEGINPDALLVPQQGVTRTPRGDASVMVVGEGDKVEVRQVTASQAIGDKWLVTDGLKSGDRVIVTGLQKIKPGVQVKAQEVASDDKQQAAGNAPSEQTKS

SEQ ID NO: 5 - DNA, 1149 bp, Pseudomonas aeruginosa
atgcaacgaacgccagccatgcgtgtactggttccggccctgctggtcgcgatttcggccctttccgggtgcggaaaaagcgaggcgccgccgccggcgcaaacgccggaggtcgggatcgtgaccctggaagcgcagacggtgaccctgaataccgagctgccgggccggaccaatgcgttccgcatcgccgaggtgcgtccccaggtgaacggcatcatcctcaagcgcctgttcaaggaaggcagcgacgtcaaggccgggcagcagctctaccagatcgaccccgccacctacgaggccgactaccagagcgcccaggccaacctggcttcgacccaggaacaggcccagcgctacaagctgctggtcgccgaccaggccgtgagcaagcagcagtacgccgacgccaatgccgcctacctgcagtccaaggcggcggtggagcaggcgcggatcaacctgcgctacaccaaggtgctgtcgccgatctccggccgcatcggccgttccgcggtgaccgaaggcgccctggtgaccaacggccaggccaacgcgatggccaccgtgcaacagctcgacccgatctacgtcgacgtcacccagccgtccaccgccctgctgcgcctgcgccgcgaactggccagcggccagttggagcgcgccggcgacaacgcggcgaaggtctccctgaagctggaggacggtagccaatacccgctggaaggtcgcctcgaattctccgaggtttccgtcgacgaaggcaccggctcggtcaccatccgcgccgtgttccccaacccgaacaacgagctgctgcccggcatgttcgttcacgcgcagttgcaggaaggcgtcaagcagaaggccatcctcgctccgcagcaaggcgtgacccgcgacctcaagggccaggctaccgcgctggtggtgaacgcgcagaacaaggtcgagctgcgggtgatcaaggccgaccgggtgatcggcgacaagtggctggttaccgaaggcctgaacgccggcgacaagatcattaccgaaggcctgcagttcgtgcagccgggtgtcgaggtgaagaccgtgccggcgaagaatgtcgcgtccgcgcagaaggccgacgccgctccggcgaaaaccgacagcaagggc

SEQ ID NO: 6 - protein, 383 aa, Pseudomonas aeruginosa
MQRTPAMRVLVPALLVAISALSGCGKSEAPPPAQTPEVGIVTLEAQTVTLNTELPGRTNAFRIAEVRPQVNGIILKRLFKEGSDVKAGQQLYQIDPATYEADYQSAQANLASTQEQAQRYKLLVADQAVSKQQYADANAAYLQSKAAVEQARINLRYTKVLSPISGRIGRSAVTEGALVTNGQANAMATVQQLDPIYVDVTQPSTALLRLRRELASGQLERAGDNAAKVSLKLEDGSQYPLEGRLEFSEVSVDEGTGSVTIRAVFPNPNNELLPGMFVHAQLQEGVKQKAILAPQQGVTRDLKGQATALVVNAQNKVELRVIKADRVIGDKWLVTEGLNAGDKIITEGLQFVQPGVEVKTVPAKNVASAQKADAAPAKTDSKG

SEQ ID NO: 7 - DNA, 1191 bp, Salmonella typhimurium
atgaacaaaaacagagggttaacgcctctggcggtcgttctgatgctctcaggcagcttagcgctaacaggatgtgacgacaaacaggaccagcaaggcggccagcagatgccagaagttggggttgtcacactaaaaacggaaccactgcaaatcacaactgaacttccgggtcgtaccgttgcttaccgtatcgccgaagttcgcccgcaggtaagcggcattatcctgaagcgtaatttcgttgagggaagtgatatcgaagcgggagtctctctctatcagattgatcctgcgacttaccaggcgacttacgacagcgctaagggcgatctggcaaaagcgcaggccgccgcgaatatcgctgaactgacggtgaagcgttatcaaaagctgctgggtacgcagtacatcagtaagcaggaatacgatcaggcgctggctgacgcgcaacaagcgactgccgccgttgtcgcagcaaaagccgccgttgaaaccgcacgtatcaacctggcgtataccaaagtcacctcaccgattagcggtcgtattggtaagtcgtccgtaacggaaggcgcgctggtacagaacggtcaggcgtcggcgctggcgacagtgcagcagctggaccctatttatgtcgatgtgacccagtccagcaatgacttcctgcgcctgaagcaggagctggcaaatggttcgctgaagcaggaaaacggcaaagcgaaggtcgatctggtgacaagcgacggtatcaaattcccgcagtccggtacgcttgaattctccgacgtgaccgttgaccaaagcaccgggtctattactttgcgcgccatcttccctaacccggatcacaccttattgccaggaatgttcgttcgcgcacgtctgcaggaagggacaaaaccgacggcattactggttccacaacagggcgttacccgtactccacgcggcgatgccacggtgctggtggttggcgctgataacaaagtggaaacccgccaaatcgtcgcaagccaggcgatcggcgataagtggctggtgactgacgggttgaaagcgggcgaccgcgtagtcgtcagcgggctgcaaaaagtacgtcctggcgcacaggttaaagtgcaggaaattaccgcggataacaaacagcaagccgcaagcggtgatcaacctgctcagcccaggtct

SEQ ID NO: 8 - protein, 397 aa, Salmonella typhimurium
MNKNRGLTPLAVVLMLSGSLALTGCDDKQDQQGGQQMPEVGVVTLKTEPLQITTELPGRTVAYRIAEVRPQVSGIILKRNFVEGSDIEAGVSLYQIDPATYQATYDSAKGDLAKAQAAANIAELTVKRYQKLLGTQYISKQEYDQALADAQQATAAVVAAKAAVETARINLAYTKVTSPISGRIGKSSVTEGALVQNGQASALATVQQLDPIYVDVTQSSNDFLRLKQELANGSLKQENGKAKVDLVTSDGIKFPQSGTLEFSDVTVDQSTGSITLRAIFPNPDHTLLPGMFVRARLQEGTKPTALLVPQQGVTRTPRGDATVLVVGADNKVETRQIVASQAIGDKWLVTDGLKAGDRVVVSGLQKVRPGAQVKVQEITADNKQQAASGDQPAQPRS

SEQ ID NO: 9 - DNA, 1200 bp, Enterobacter aerogenes
atgaacaaaaacagagggttaacgcctctggcggtcgttctgatgctctcaggcagcttagcgctaacaggatgtgacgataaaccggctcaacaaggagcccagcaaatgccggaagtcggtattgtcacgctcaaatccgcacctctacaaatcaccactgaacttcctggtcgtaccaatgcttatcgtgtcgcagaagtccgtcctcaggtaagtggcattattctgaaacgtaacttcactgaaggcagcgatattcaggcgggggtgtctctttatcagatcgatccggcaacctatcaggcaagctatgaaagcgctaaaggcgacttagcgaaagcccaggccgccgcaaacatcgctcagttgacggtcaaacgttatcagaaactggttggcaccaaatacatcagccaacaagaatatgactcagccgttgccgacgctcagcaaagcaacgccgccgtcgtcgcggcgaaagccgcagttgaaaccgcgcgtatcaacctggcttataccaaagtgacgtcgccaatcagtggacgcatcggtaaatccgcggtcaccgaaggcgcgctggtgcaaaacgggcaatctaccgcgctggctaccgttcagcagctagaccctatctatgttgacgttacccaatcgagcaacgatttcctgcgtctgaaacaggaactggcgaacggtaaactgaagcaagaaaacggtaaggcgaaagttgaactggtcaccaatgacggtctgaaatatccgcaagagggtacgctggaattctccgacgtcaccgttgaccagaccaccggttctatcacgctgcgcgctatcttcccgaacccggaccacactctgctgccaggaatgtttgttcgcgcacgtctggaagaaggtattaacccggatgcgctgctggttccgcaacagggcgttacccgtaccccgcgcggcgatgccagcgtgatggtggtcggcgaaggcgacaaagttgaagtgcgccaggtcaccgcgactcaggctatcggcgacaagtggttagtgaccgaaggtctgaaatcaggcgatcgcgtgatcgtcgctggcctgcaaaaagtcaaaccaggcgtacaggtaaaagcgcaggaagtagcttcagacaaccaacagcaaaccgccggcaacgcgaacgctcagtctgaacaaaccaagtcttaa

SEQ ID NO: 10 - protein, 399 aa, Enterobacter aerogenes
MNKNRGLTPLAVVLMLSGSLALTGCDDKPAQQGAQQMPEVGIVTLKSAPLQITTELPGRTNAYRVAEVRPQVSGIILKRNFTEGSDIQAGVSLYQIDPATYQASYESAKGDLAKAQAAANIAQLTVKRYQKLVGTKYISQQEYDSAVADAQQSNAAVVAAKAAVETARINLAYTKVTSPISGRIGKSAVTEGALVQNGQSTALATVQQLDPIYVDVTQSSNDFLRLKQELANGKLKQENGKAKVELVTNDGLKYPQEGTLEFSDVTVDQTTGSITLRAIFPNPDHTLLPGMFVRARLEEGINPDALLVPQQGVTRTPRGDASVMVVGEGDKVEVRQVTATQAIGDKWLVTEGLKSGDRVIVAGLQKVKPGVQVKAQEVASDNQQQTAGNANAQSEQTKS

SEQ ID NO: 11 - DNA, 3150 bp, Escherichia coli
atgcctaatttctttatcgatcgcccgatttttgcgtgggtgatcgccattatcatcatgttggcaggggggctggcgatcctcaaactgccggtggcgcaatatcctacgattgcaccgccggcagtaacgatctccgcctcctaccccggcgctgatgcgaaaacagtgcaggacacggtgacacaggttatcgaacagaatatgaacggtatcgataacctgatgtacatgtcctctaacagtgactccacgggtaccgtgcagatcaccctgacctttgagtctggtactgatgcggatatcgcgcaggttcaggtgcagaacaaactgcagctggcgatgccgttgctgccgcaagaagttcagcagcaaggggtgagcgttgagaaatcatccagcagcttcctgatggttgtcggcgttatcaacaccgatggcaccatgacgcaggaggatatctccgactacgtggcggcgaatatgaaagatgccatcagccgtacgtcgggcgtgggtgatgttcagttgttcggttcacagtacgcgatgcgtatctggatgaacccgaatgagctgaacaaattccagctaacgccggttgatgtcattaccgccatcaaagcgcagaacgcccaggttgcggcgggtcagctcggtggtacgccgccggtgaaaggccaacagcttaacgcctctattattgctcagacgcgtctgacctctactgaagagttcggcaaaatcctgctgaaagtgaatcaggatggttcccgcgtgctgctgcgtgacgtcgcgaagattgagctgggtggtgagaactacgacatcatcgcagagtttaacggccaaccggcttccggtctggggatcaagctggcgaccggtgcaaacgcgctggataccgctgcggcaatccgtgctgaactggcgaagatggaaccgttcttcccgtcgggtctgaaaattgtttacccatacgacaccacgccgttcgtgaaaatctctattcacgaagtggttaaaacgctggtcgaagcgatcatcctcgtgttcctggttatgtatctgttcctgcagaacttccgcgcgacgttgattccgaccattgccgtaccggtggtattgctcgggacctttgccgtccttgccgcctttggcttctcgataaacacgctaacaatgttcgggatggtgctcgccatcggcctgttggtggatgacgccatcgttgtggtagaaaacgttgagcgtgttatggcggaagaaggtttgccgccaaaagaagctacccgtaagtcgatggggcagattcagggcgctctggtcggtatcgcgatggtactgtcggcggtattcgtaccgatggccttctttggcggttctactggtgctatctatcgtcagttctctattaccattgtttcagcaatggcgctgtcggtactggtggcgttgatcctgactccagctctttgtgccaccatgctgaaaccgattgccaaaggcgatcacggggaaggtaaaaaaggcttcttcggctggtttaaccgcatgttcgagaagagcacgcaccactacaccgacagcgtaggcggtattctgcgcagtacggggcgttacctggtgctgtatctgatcatcgtggtcggcatggcctatctgttcgtgcgtctgccaagctccttcttgccagatgaggaccagggcgtgtttatgaccatggttcagctgccagcaggtgcaacgcaggaacgtacacagaaagtgctcaatgaggtaacgcattactatctgaccaaagaaaagaacaacgttgagtcggtgttcgccgttaacggcttcggctttgcgggacgtggtcagaataccggtattgcgttcgtttccttgaaggactgggccgatcgtccgggcgaagaaaacaaagttgaagcgattaccatgcgtgcaacacgcgctttctcgcaaatcaaagatgcgatggttttcgcctttaacctgcccgcaatcgtggaactgggtactgcaaccggctttgactttgagctgattgaccaggctggccttggtcacgaaaaactgactcaggcgcgtaaccagttgcttgcagaagcagcgaagcaccctgatatgttgaccagcgtacgtccaaacggtctggaagataccccgcagtttaagattgatatcgaccaggaaaaagcgcaggcgctgggtgtttctatcaacgacattaacaccactctgggcgctgcatggggcggcagctatgtgaacgactttatcgaccgcggtcgtgtgaagaaagtttatgtcatgtcagaagcgaaataccgtatgctgccggatgatatcggcgactggtatgttcgtgctgctgatggtcagatggtgccattctcggcgttctcctcttctcgttgggagtacggttcgccgcgtctggaacgttacaacggcctgccatccatggaaatcttaggccaggcggcaccgggtaaaagtaccggtgaagcaatggagctgatggaacaactggcgagcaaactgcctaccggtgttggctatgactggacggggatgtcctatcaggaacgtctctccggcaaccaggcaccttcactgtacgcgatttcgttgattgtcgtgttcctgtgtctggcggcgctgtacgagagctggtcgattccgttctccgttatgctggtcgttccgctgggggttatcggtgcgttgctggctgccaccttccgtggcctgaccaatgacgtttacttccaggtaggcctgctcacaaccattgggttgtcggcgaagaacgcgatccttatcgtcgaattcgccaaagacttgatggataaagaaggtaaaggtctgattgaagcgacgcttgatgcggtgcggatgcgtttacgtccgatcctgatgacctcgctggcgtttatcctcggcgttatgccgctggttatcagtactggtgctggttccggcgcgcagaacgcagtaggtaccggtgtaatgggcgggatggtgaccgcaacggtactggcaatcttcttcgttccggtattctttgtggtggttcgccgccgctttagccgcaagaatgaagatatcgagcacagccatactgtcgatcatcattga

SEQ ID NO: 12 - protein, 1049 aa, Escherichia coli [sequence omitted]

SEQ ID NO: 13 - DNA, 3147 bp, Klebsiella pneumoniae
atgcctaatttctttatcgatcgccccatatttgcatgggtgatcgccattatcatcatgctggctgggggactatcgatcctcaaattgccggtggcgcaatatccgacgattgcgccgccagcaatttccatttccgccatgtaccccggtgctgacccccaaacagtgcagaacaccgtaacacaggttatcgaacagaatatgaacggtatcgatcacctgatgtacatgtcctcaaatggggactccaccggtacggtaaacattacgctgaccttcgaatccggtaccgatccggatatcgcccatgttcaggttcagaacaagctggcgctggcgacgcctctgctgccgcaagaagtacagcagcaagggattagcgttgagaaagcgtccagcagcttcctgatggttgtcggcgtcattaacaccaacggcaccatgaaccaggacgatatttcggactacgtggcggccaacatgaaggatccgattagccgtaccagcggcgtcggcgacgttcagctattcggttcccagtatgcgatgcgtatctggatggatccaaacaaactgaacaactttcagcttacgccggtggatgtgatcagcgccctgaaagcgcagaacgcccaggtggccgcgggccagttaggcggtacgccgccggtgaaaggccagcagcttaacgcctcgatcatcgcgcagacgcgtctgaccaatacggaagagtttggcaacatcctgctgaaggtgaaccaggacggttcccaggttcgtctgcgtgatgtcgccaaaattgagctgggcggcgaaagctatgacgtagtggcgaagtttaacggccagccggcatccggtctgggtattaaactggctaccggcgcgaacgcgctggataccgccaacgctattcgcgctgaactggcgaagatggagccgtttttcccgtcggggatgaagatcgtttacccgtatgacaccaccccgttcgtgaaaatttctattcacgaagtggttaaaacgctggtggaagcgatcatcctggtgttcctggtcatgtatctgttcctgcagaacttccgcgccaccctgatccccaccatcgcggtaccggtggtcctgttaggcaccttcgcggtgctggcggcgtttggcttctcgataaacaccctgacgatgttcgggatggtgctcgccatcggcctgttggtggatgacgccatcgtggtggtagagaacgtcgagcgcgtgatggcggaagagggtctgccgccgaaagaagcgacgcgtaaatcgatgggacagattcagggcgcgctggttggtatcgcgatggtgctgtcggcggtatttatcccgatggcgttcttcggcggctcaaccggggccatctatcgccagttctccatcactatcgtttccgcgatggcgctgtcggtactggtggcgttaatcctgacgccggcgctgtgcgccacgatgctgaaacccattcagaaaggcagccatggcgcgaccaccggcttcttcggctggtttaaccgcatgtttgataagagcacgcaccattacaccgacagcgtaggcaacattctgcgcagcaccggtcgttatctggtgctgtatctgatcatcgtggtgggtatggcatggctgttcgtccgtctgccgagctcgttcctgccggacgaggaccagggtgtattcctgagtatggcgcagctgcctgccggcgccacccaggagcgtacgcagaaagtgctggatgagatgacgaattactatctcaccaaagagaaggacaacgtggaatccgtgtttgcggttaacggcttcggtttcgccggccgcggccagaacaccggtatcgcgttcgtttcgctgaaagactggagccagcgtccgggtgaggaaaacaaagttgaagcgatcaccgctagagcaatgggctacttctcgcagattaaagatgcgatggtcttcgcctttaacctgccggctatcgttgaactgggtaccgcgaccggctttgacttcgagctgattgaccaggcgggtctgggccacgaaaaactgacccaggcgcgtaaccaactgtttggcatggtggcgcagcatcctgacgtgctgaccggcgtgcgccctaacggtctggaagatacaccgcagtttaaaatcgatatcgatcaggagaaagctcaggcgctgggcgtctccatcagcgacattaacaccacgctgggcgcagcctggggcggaagctatgtcaacgactttatcgaccgcggccgcgtgaagaaagtgtacatcatgtccgaagcgaaataccgtatgctgccggaagatatcggcaagtggtatgttcgcggcagcgatggtcagatggtgccgttctctgctttctcgacctcgcgttgggaatacggttcgccgcgtctggaacgctacaacggtctgccgtcactggaaatcctcggtcaggccgcgccaggcaagagtaccggtgaggcgatggcgctgatggaagagctggcgggtaaactgccttccggtatcggctacgactggaccgggatgtcttatcaggagcgactgtccggcaaccaggcccctgccctgtatgccatctcgctgattgtcgtcttcctgtgtctggcggcgctgtatgagagctggtcgattccgttctcggttatgctggtcgtccccttgggtgtggtcggtgcgctgttagccgccaccttccgcgggctaaccaacgacgtttacttccaggtgggtctgctgaccaccatcggcctgtcggcgaagaacgcaatattgatcgttgaattcgccaaagacctgatggagaaagagggcaaagggctgattgaggcgacgcttgaagcagtacgtatgcgtctacgtccaatcctgatgacatctctggcgttcatcctcggggtaatgccgctagttatcagctccggcgccggctccggtgcgcagaacgccgtcggtacaggcgtaatgggcgggatggtgaccgcgacgatcctggcgatcttcttcgtgccggtgttctttgtggtggttcgtcgccgctttagcaaaaaatcggaagatatagagcacagccatcaggttgagcatcattaa

SEQ ID NO: 14 - protein, 1048 aa, Klebsiella pneumoniae
MPNFFIDRPIFAWVIAIIIMLAGGLSILKLPVAQYPTIAPPAISISAMYPGADPQTVQNTVTQVIEQNMNGIDHLMYMSSNGDSTGTVNITLTFESGTDPDIAHVQVQNKLALATPLLPQEVQQQGISVEKASSSFLMVVGVINTNGTMNQDDISDYVAANMKDPISRTSGVGDVQLFGSQYAMRIWMDPNKLNNFQLTPVDVISALKAQNAQVAAGQLGGTPPVKGQQLNASIIAQTRLTNTEEFGNILLKVNQDGSQVRLRDVAKIELGGESYDVVAKFNGQPASGLGIKLATGANALDTANAIRAELAKMEPFFPSGMKIVYPYDTTPFVKISIHEVVKTLVEAIILVFLVMYLFLQNFRATLIPTIAVPVVLLGTFAVLAAFGFSINTLTMFGMVLAIGLLVDDAIVVVENVERVMAEEGLPPKEATRKSMGQIQGALVGIAMVLSAVFIPMAFFGGSTGAIYRQFSITIVSAMALSVLVALILTPALCATMLKPIQKGSHGATTGFFGWFNRMFDKSTHHYTDSVGNILRSTGRYLVLYLIIVVGMAWLFVRLPSSFLPDEDQGVFLSMAQLPAGATQERTQKVLDEMTNYYLTKEKDNVESVFAVNGFGFAGRGQNTGIAFVSLKDWSQRPGEENKVEAITARAMGYFSQIKDAMVFAFNLPAIVELGTATGFDFELIDQAGLGHEKLTQARNQLFGMVAQHPDVLTGVRPNGLEDTPQFKIDIDQEKAQALGVSISDINTTLGAAWGGSYVNDFIDRGRVKKVYIMSEAKYRMLPEDIGKWYVRGSDGQMVPFSAFSTSRWEYGSPRLERYNGLPSLEILGQAAPGKSTGEAMALMEELAGKLPSGIGYDWTGMSYQERLSGNQAPALYAISLIVVFLCLAALYESWSIPFSVMLVVPLGVVGALLAATFRGLTNDVYFQVGLLTTIGLSAKNAILIVEFAKDLMEKEGKGLIEATLEAVRMRLRPILMTSLAFILGVMPLVISSGAGSGAQNAVGTGVMGGMVTATILAIFFVPVFFVVVRRRFSKKSEDIEHSHQVEHH

SEQ ID NO: 15 - DNA, 3138 bp, Pseudomonas aeruginosa
atgtcgaagtttttcattgataggcccattttcgcgtgggtgatcgccttggtgatcatgctcgcgggcggcctgtcgatcctcagtctgccggtcaaccagtacccggccatcgccccgccggccatcgccgtgcaggtgagctacccgggcgcctcggccgagacggtgcaggacaccgtggtccaggtgatcgagcagcagatgaacgggatcgacaatctgcgctacatctcctcggagagtaactccgacggcagcatgaccatcaccgtgaccttcgaacagggcaccgaccccgacatcgcccaggtccaggtgcagaacaagctgcaactggccaccccgctactgccgcaggaagtgcagcgccaggggatccgggtgaccaaggcggtgaagaacttcctcatggtggtcggtgtggtttccaccgacggcagcatgaccaaggaagacctgtcgaactacatcgtttccaacatccaggacccactctcgcggaccaagggcgtcggtgacttccaggtgttcggctcgcagtactcgatgcgcatctggctcgacccggccaagctgaacagctaccagctgacccccggcgacgtgagcagcgcgatccaggcgcagaacgtgcagatttcctccggccagctcggcggcttgccggcggtcaagggccagcagctcaacgccaccatcatcggcaagacccgcctgcagaccgcggagcaattcgagaacatcctgctcaaggtcaatcccgacggttcccaggtgcgcctgaaggacgtcgccgatgtaggcctgggcggccaggactacagcatcaacgcgcagttcaacggcagcccggcgtccggtatcgcgatcaagctggccaccggcgccaacgcgctggataccgccaaggcgatccgccagaccatcgccaacctggaaccgttcatgccgcagggcatgaaggtggtctacccgtacgacaccaccccggtggtctcggcctcgatccatgaggtagtgaagaccctcggcgaggcgatcctcctcgtgttcctggtgatgtacctgttcctgcagaacttccgcgccacgctgatcccgaccatcgccgtaccggtggtgctgctggggaccttcggcgtgctcgccgcgttcggcttctcgatcaacaccctgaccatgttcggcatggtgctggccatcggcttgctggtggacgacgccatcgtggtggtggagaacgtcgagcgggtgatggccgaggaaggcctgtcgccaagggaggcggcgcgcaagtccatgggccagatccagggcgcgctggtcggtatcgccatggtgctctcggcggtattcctgccgatggcgttcttcggcggctccaccggggtgatctaccggcagttctccatcaccatcgtgtcggccatggccctctcggtgatcgtggcgctgatcctcaccccggcgctctgcgcgaccatgctcaagccgatcgagaaaggcgaccatggcgagcacaagggcggcttcttcggctggttcaaccggatgttcctttccaccacccacggctacgagcggggcgtggcgtcgatcctcaagcatcgcgcgccgtacctgctgatctacgtggtgatcgtggccgggatgatctggatgttcacccgcattcccaccgcgttcctccccgacgaggaccagggcgtactgttcgcccaggtacagaccccgccgggctccagtgccgagcgtacccaggtggtggtggactcgatgcgcgaatacctgctggagaaggaaagctcttcggtcagctcggtgttcaccgtgaccggcttcaacttcgccggccgcggccagagttcgggcatggcgttcatcatgctcaagccctgggaagagcgtcccggtggcgagaacagcgtgttcgaactggccaagcgcgcgcagatgcacttcttcagcttcaaggacgcgatggtgttcgccttcgcgccgccgtcggtactggaactgggtaacgccaccggcttcgacctgttcctccaggaccaggcgggtgtcggccacgaagtcctgctccaggcgcgcaacaagttcctcatgctcgccgcgcagaacccggcgctgcaacgcgtgcgccccaacggcatgagcgacgaaccgcagtacaagctggagatcgacgacgagaaggccagcgccctcggcgtgtcccttgccgacatcaacagcaccgtgtccatcgcctggggttccagctacgtcaacgatttcatcgaccgtggccgggtcaagcgggtctacctgcagggcaggccggacgcgcggatgaacccggacgacctgagcaagtggtacgtgcgcaacgacaagggcgagatggtgccgttcaacgccttcgccaccggcaagtgggaatacggttcgccgaagctggagcgctacaatggcgtgccggcgatggagatcctcggcgagccggcgcccggcctgagttccggtgacgccatggcggcggtcgaggagatcgtcaagcaattgccgaaaggcgttggctactcctggaccggcctgtcctacgaggagcgcttgtccggctcgcaggcgccggcgctgtatgcgctgtcgctgctggtggtgttcctctgcctggcggccctgtacgaaagctggtcgattccgttctcggtgatgctggtggtgccgttgggcgtgatcggtgcgctgctggcgacgtccatgcgcggcctgtccaacgacgtgttcttccaggtgggcctgttgacgaccatcggcctgtcggcgaagaacgccattctcatcgtggagttcgccaaggagctgcacgagcagggcaagggcatcgtcgaggcggccatcgaagcctgccgcatgcgtctgcggccgatcgtgatgacctccctggcgttcatcctcggcgtggtcccgctggcgatctccaccggcgccggctcgggcagccagcatgcgatcggtaccggcgtgatcggcggcatggtcactgcgaccgtcctggcgatcttctgggtaccgctgttctacgtggcggtcagcacgctgttcaaggacgaggcgtccaagcagcaggcgtccgtcgaaaaggggcaa

SEQ ID NO: 16 - protein, 1046 aa, Pseudomonas aeruginosa
MSKFFIDRPIFAWVIALVIMLAGGLSILSLPVNQYPAIAPPAIAVQVSYPGASAETVQDTVVQVIEQQMNGIDNLRYISSESNSDGSMTITVTFEQGTDPDIAQVQVQNKLQLATPLLPQEVQRQGIRVTKAVKNFLMVVGVVSTDGSMTKEDLSNYIVSNIQDPLSRTKGVGDFQVFGSQYSMRIWLDPAKLNSYQLTPGDVSSAIQAQNVQISSGQLGGLPAVKGQQLNATIIGKTRLQTAEQFENILLKVNPDGSQVRLKDVADVGLGGQDYSINAQFNGSPASGIAIKLATGANALDTAKAIRQTIANLEPFMPQGMKVVYPYDTTPVVSASIHEVVKTLGEAILLVFLVMYLFLQNFRATLIPTIAVPVVLLGTFGVLAAFGFSINTLTMFGMVLAIGLLVDDAIVVVENVERVMAEEGLSPREAARKSMGQIQGALVGIAMVLSAVFLPMAFFGGSTGVIYRQFSITIVSAMALSVIVALILTPALCATMLKPIEKGDHGEHKGGFFGWFNRMFLSTTHGYERGVASILKHRAPYLLIYVVIVAGMIWMFTRIPTAFLPDEDQGVLFAQVQTPPGSSAERTQVVVDSMREYLLEKESSSVSSVFTVTGFNFAGRGQSSGMAFIMLKPWEERPGGENSVFELAKRAQMHFFSFKDAMVFAFAPPSVLELGNATGFDLFLQDQAGVGHEVLLQARNKFLMLAAQNPALQRVRPNGMSDEPQYKLEIDDEKASALGVSLADINSTVSIAWGSSYVNDFIDRGRVKRVYLQGRPDARMNPDDLSKWYVRNDKGEMVPFNAFATGKWEYGSPKLERYNGVPAMEILGEPAPGLSSGDAMAAVEEIVKQLPKGVGYSWTGLSYEERLSGSQAPALYALSLLVVFLCLAALYESWSIPFSVMLVVPLGVIGALLATSMRGLSNDVFFQVGLLTTIGLSAKNAILIVEFAKELHEQGKGIVEAAIEACRMRLRPIVMTSLAFILGVVPLAISTGAGSGSQHAIGTGVIGGMVTATVLAIFWVPLFYVAVSTLFKDEASKQQASVEKGQ

SEQ ID NO: 17 - DNA, 3144 bp, Salmonella typhimurium
atgttctgtcgaatgactatgctcaatatcttcgcttttacggctaaagcggcggcgtaccaccacgaagaagactggtacgaagaaaatagccagtacggttgccgttaccatcccgcccagtacgccagtacctaccgcattctgcgcgccggaacccgcgccggaactgataaccagcggcataacccccagcatgaacgctaacgaggtcatcagaatcggacgcaaacgcatccggacggcctccagcatcgcttctaccagacctttcccttctttatccattaagtctttggcgaattcgacgataagtatcgcgttcttcgccgacaacccaatggttgtgagcaggcccacctggaagtaaacgtcgttagtcagtccgcggaaggtcgcggccagcagcgcgccgataaccccaagcggaacaaccagcattacggagaacgggatagaccagctctcatacaatgccgccagacacaggaagacgacgattagcgagatagcatacagggcaggggcctggttgccggacaaccgctcctggtaggacatcccggtccagtcatacccaatgcctgacggcagcttgctggccagttcttccatcatcgccatcgcttcaccagtactcttgcctggcgccgcctgacccagaatttccatcgaaggcagaccgttatagcgttccagacgcggcgaaccatattcccagcgggaagaggagaatgcggagaacggcaccatctgaccatcgctaccacgaacgtaccagtcgttaatatcatccggcaacatgcggtatttcgcttcggacatcacgtaaactttcttcacacgaccgcgatcgataaagtcgtttacatagctgccgccccatgctgcgcccagcgtggtattaatgtcgctaatagacacgcccagcgcctgagctttttcctggtcgatatcgattttaaactgcggcgtatcttccagaccgttaggtcgaacgccgaccaacagatcaggatatttcgccacctcgccgaacaactgattacgtgcctgggtgagtttttcgtgaccaagtcccgcctggtcaatcaactcgaagtcaaacccggttgcggtgcccagttcaacaatcgccggtaggttaaaggcgaagaccatcgcatctttaatttgtgaaaacgctgcggttgcccgctgggtaatcgcttcaaccttgtttttttcgcctggacgatcggcccagtctttcaacgacacaaatgcaataccggtattctgaccgcgccctgcaaaaccgaagccgttgacggcgaataccgattcaacgttggctttctctttgttcagatagtaatccgtgacctcatccagcactttttgcgtgcgctcttgcgtcgcgccggcggggagctggaccattgtcaggaatacgccctggtcttcatccggcaagaaagagcttggcagacgaacgaacagataagccataccgacgacgataatgagatagagcagcagataacgcccggtgctgcgcagaatattccctacgctatcggtgtagtgatgcgtgctcttatcaaacaggcggttaaaccagccgaaaaagccttttttcccttcgccatgatcgcctttggcgacgggtttgagcatcgtcgcgcacagcgcgggcgtcaggatcagcgcgaccagcaccgacagcgccatcgccgatacgatggtgatagagaactgacgataaattgccccggttgagccgccaaagaaggccatcggaataaataccgccgacagtaccatcgcgatacccaccaatgcgccctgaatctggcccatggatttgcgcgtcgcttccttcggcggaaggccttcttccgtcataacacgttcgacgttctcgaccaccacgatggcgtcatccaccagcaagccgatggcgagcaccatcccgaacatcgtcagcgtgtttatcgagaaaccgaatgccgcaagcacggcaaaggttcccaacaacaccaccggaaccgcaatagtcggaatcaacgtcgcgcggaagttctgcaggaacaggtacatcaccaagaacacgaggataatcgcttcgaccagcgtttttaccacttcatgaatagagatcttcacgaacggcgtggtgtcatacgggtagacgattttcatccctggcgggaagaacggttccatttttttcagttcggcgcgaatagcggtagcggtatccagcgcgttggcgccggtagccagtttgatgccaagacctgacgctggctgaccgttaaatttcgcgatgacgtcgtagttttcgccgccaagctcaattttcgctacatcccgcagacgaacctgagagccatcctgattcactttcagcaggattttgccaaactcatccgttgaggtcagacgcgtttgggcaataatcgatgcgttaagctgctggccttttaccggcggtgtaccaccgagctgacctgccgcgacctgggcgttctgcgctttgatcgcgttaatcacgtcgaccggcgtcagttggtatttggtcagctctgtcggattcatccagatacgcatcgcatactgcgaaccaaacagctggacgtcgcccaccccagaggtacggctgatcggatctttcatattggcggcaacgtaatccgaaatatcctcctgggtcatggtgccgtcggtgttaatgacgcccactaccatcaggaagctacttgaggacttctcaacgctcacgccctgttgctgtacttcctggggaagtaacggcattgccagttgcaacttgttctgaacctgaacctgcgcgatatccgcatcggtgccggattcaaaggtcagcgtgatctgtacggtccccgtggagtcactgttggaggacatatacatcaggttatcgataccgttcatattctgttcgataacctgcgtgacggtatcctgtaccgttttcgcatcagcgccagggtaggttgcggagatcgtcactgctggtggcgcaatcgtcggatattgcgctaccggcaatttgaggatcgcgagcccccctgccaacatgatgatgatggcgatcacccacgcaaatatagggcgatcgataaagaaattaggcat

SEQ ID NO: 18 - protein, 1048 aa, Salmonella typhimurium
MPNFFIDRPIFAWVIAIIIMLAGGLAILKLPVAQYPTIAPPAVTISATYPGADAKTVQDTVTQVIEQNMNGIDNLMYMSSNSDSTGTVQITLTFESGTDADIAQVQVQNKLQLAMPLLPQEVQQQGVSVEKSSSSFLMVVGVINTDGTMTQEDISDYVAANMKDPISRTSGVGDVQLFGSQYAMRIWMNPTELTKYQLTPVDVINAIKAQNAQVAAGQLGGTPPVKGQQLNASIIAQTRLTSTDEFGKILLKVNQDGSQVRLRDVAKIELGGENYDVIAKFNGQPASGLGIKLATGANALDTATAIRAELKKMEPFFPPGMKIVYPYDTTPFVKISIHEVVKTLVEAIILVFLVMYLFLQNFRATLIPTIAVPVVLLGTFAVLAAFGFSINTLTMFGMVLAIGLLVDDAIVVVENVERVMTEEGLPPKEATRKSMGQIQGALVGIAMVLSAVFIPMAFFGGSTGAIYRQFSITIVSAMALSVLVALILTPALCATMLKPVAKGDHGEGKKGFFGWFNRLFDKSTHHYTDSVGNILRSTGRYLLLYLIIVVGMAYLFVRLPSSFLPDEDQGVFLTMVQLPAGATQERTQKVLDEVTDYYLNKEKANVESVFAVNGFGFAGRGQNTGIAFVSLKDWADRPGEKNKVEAITQRATAAFSQIKDAMVFAFNLPAIVELGTATGFDFELIDQAGLGHEKLTQARNQLFGEVAKYPDLLVGVRPNGLEDTPQFKIDIDQEKAQALGVSISDINTTLGAAWGGSYVNDFIDRGRVKKVYVMSEAKYRMLPDDINDWYVRGSDGQMVPFSAFSSSRWEYGSPRLERYNGLPSMEILGQAAPGKSTGEAMAMMEELASKLPSGIGYDWTGMSYQERLSGNQAPALYAISLIVVFLCLAALYESWSIPFSVMLVVPLGVIGALLAATFRGLTNDVYFQVGLLTTIGLSAKNAILIVEFAKDLMDKEGKGLVEAMLEAVRMRLRPILMTSLAFMLGVMPLVISSGAGSGAQNAVGTGVLGGMVTATVLAIFFVPVFFVVVRRRFSRKSEDIEHSHSTEH

SEQ ID NO: 19 - DNA, 3147 bp, Enterobacter aerogenes
atgcctaatttctttatcgatcgccccatatttgcatgggtgatcgccataatcatcatgttggcaggggggctttcgattatgaaactccccgtggcgcaatatccaacgattgcgccgccagcagtgacgatctcggccacctatccgggggctgatgcgaaaacggtgcaggacaccgtgacacaggttatcgaacagaacatgaacggtatcgataacctgatgtacatgtcctcgaacagtgactccaccggtacggtgcagatcaccctgaccttccagtccggtaccgacgcagatatcgcccaggtacaggtgcagaataagctgcagctggcgatgccgctgctgccgcaggaagtacaacaacagggcgtgagcgtggagaaatcctccagtagcttcctgatggttgtcggggttatcaacactaacggcaccatgacgcaggaggatatttccgactacgtgggcgccaatatgaaggatgccatcagccgtacttccggcgtcggcgacgttcagctgttcggttcccagtacgcaatgcgtatctggatggacccgacgaagctaaacaacttccagttaactccggtagatgtcatcaacgccattaaagcgcagaacgcccaggtcgcggcaggtcagttaggcggtacgccgccggtgaaaggccagcagttgaacgcctcgatcatcgcccaaacgcgtctgacttccgctgatgaattcagcaagattctgctgaaagtcaatcaggatggttcacaggttcgtctgcgcgacgtggcgaaagttgaactcggcggcgaaaactacgacatcatcgcgaagttcaacggccaaccggcttctggtctggggattaaactggccaccggcgctaacgccctggataccgccaacgccatccgcgccgagctggcgaagatggaaccgtacttcccgtcaggcctgaaaatcgtctacccgtacgatactaccccgttcgtgaaaatctctattcatgaagtggtaaaaacgctggtggaagcgatcatcctggtgttcctggtcatgtatctgttcctgcagaacttccgcgccacgctgatcccgaccattgcagtaccggtggtattgcttgggacattcgccatcctggcggtgtttggcttctcgataaacaccctgacgatgttcgggatggtgctggccatcggcttgctggtggatgacgccatcgtggtagtagaaaacgtcgagcgcgtaatggcggaggaaggattgccgccgaaagaagcgacccgtaagtcgatgggtcagattcagggcgcgttggtcggtattgcgatggtactgtcggcggtatttatcccgatggccttcttcggcggttcaaccggcgcgatttatcgccagttctcgatcacaatcgtttccgcgatggcgctatcggtactggtagcgctgattctgacgccagcgctgtgcgccaccatgctgaagccgattcagaaaggcggccatggcgagcacaaaggcttctttggctggtttaaccgcatgttcgataagagcacgcaccactacaccgacagcgtaggcaacattctgcgtagtaccggtcgttacctggtgctgtatctgattatcgtcgtcggtatggcttacctgttcgttcgtctgccaagctccttcctgccggacgaagaccagggcgtgttcctgagtatggcgcagcttccggcgggcgcgagtcaggaacgtacgcagaaagtcctcgacgagatgactgactactacctgaccaaagagaagaacaacgttgaatcggtgttcgcggttaacggttttggttttgcaggccgcggtcagaacaccggtatcgccttcgtgtcgttaaaagactggagcgaacgcccaggttcagaaaacaaagttgaagcgattaccggccgcgcgatggcccgtttctcgcagattaaagatgcgatggtcttcgcgtttaacctgccggcgatcgtcgaactgggtaccgctaccggctttgacttccagctcatcgatcagggtggtttagggcatgagaaattaactcaggcgcgtaaccagctgtttggtatggttgctcagcatcccgatctgctggttggcgtgcgtcctaacggcctggaagatacgccgcagttcaagatcgatatcgatcaggaaaaagcgcaggcgctgggcgtttcaattagcgacatcaacaccaccctcggcgcagcgtggggcggcagctacgtcaacgacttcatcgaccgcggtcgcgtgaagaaagtgtacgtcatgtctgaagcgaaataccgtatgctgccagaggatatcggcaactggtatgtacgcggcagcgatggccaaatggtgccgttctccgccttctccacttcccattgggaatatggttcaccgcgtctggaacgttacaacggtctgccgtcgatggaaatcctcggtcaggcagcgccgggccgcagtacgggtgaagcgatggcgatgatggaacagctggcaagcaaactgccttcaggcgtcggttacgactggaccggtatgtcctatcaggaacgcctgtcaggcaaccaggccccggcgctgtatgctatctcgcttatcgtcgtcttcctgtgtctggcagcattgtacgagagctggtcgattccgttctccgtcatgctggtggtaccgttgggtgttgttggcgcgctgttagcggcaaccttccgcgggctgactaacgacgtttacttccaggtcggcctgctgacgaccatcggcctgtcggcgaagaacgcgatacttatcgttgaattcgccaaagacctgatggaaaaagaaggcaaaggcctgattgaagcgacgctggaagcggtacgtatgcgtctgcgtccaatcctgatgacctctctggcgtttatcctcggggtcatgccgctggttatcagctctggcgcaggttccggcgcacagaacgccgtcggtaccggcgtaatgggcgggatggtgaccgcgacggttctggctatcttcttcgtgccggtgttcttcgtggtggttcgtcgccgcttcactaagaaaactgaagatattgagcataaccatccggttgagcatcattaa

SEQ ID NO: 20 - protein, 1048 aa, Enterobacter aerogenes
MPNFFIDRPIFAWVIAIIIMLAGGLSIMKLPVAQYPTIAPPAVTISATYPGADAKTVQDTVTQVIEQNMNGIDNLMYMSSNSDSTGTVQITLTFQSGTDADIAQVQVQNKLQLAMPLLPQEVQQQGVSVEKSSSSFLMVVGVINTNGTMTQEDISDYVGANMKDAISRTSGVGDVQLFGSQYAMRIWMDPTKLNNFQLTPVDVINAIKAQNAQVAAGQLGGTPPVKGQQLNASIIAQTRLTSADEFSKILLKVNQDGSQVRLRDVAKVELGGENYDIIAKFNGQPASGLGIKLATGANALDTANAIRAELAKMEPYFPSGLKIVYPYDTTPFVKISIHEVVKTLVEAIILVFLVMYLFLQNFRATLIPTIAVPVVLLGTFAILAVFGFSINTLTMFGMVLAIGLLVDDAIVVVENVERVMAEEGLPPKEATRKSMGQIQGALVGIAMVLSAVFIPMAFFGGSTGAIYRQFSITIVSAMALSVLVALILTPALCATMLKPIQKGGHGEHKGFFGWFNRMFDKSTHHYTDSVGNILRSTGRYLVLYLIIVVGMAYLFVRLPSSFLPDEDQGVFLSMAQLPAGASQERTQKVLDEMTDYYLTKEKNNVESVFAVNGFGFAGRGQNTGIAFVSLKDWSERPGSENKVEAITGRAMARFSQIKDAMVFAFNLPAIVELGTATGFDFQLIDQGGLGHEKLTQARNQLFGMVAQHPDLLVGVRPNGLEDTPQFKIDIDQEKAQALGVSISDINTTLGAAWGGSYVNDFIDRGRVKKVYVMSEAKYRMLPEDIGNWYVRGSDGQMVPFSAFSTSHWEYGSPRLERYNGLPSMEILGQAAPGRSTGEAMAMMEQLASKLPSGVGYDWTGMSYQERLSGNQAPALYAISLIVVFLCLAALYESWSIPFSVMLVVPLGVVGALLAATFRGLTNDVYFQVGLLTTIGLSAKNAILIVEFAKDLMEKEGKGLIEATLEAVRMRLRPILMTSLAFILGVMPLVISSGAGSGAQNAVGTGVMGGMVTATVLAIFFVPVFFVVVRRRFTKKTEDIEHNHPVEHH

SEQ ID NO: 21 - DNA, 1158 bp, Escherichia coli
atgacgaaacatgccaggtttttcctcctgccctcctttattctgatctccgcggctttaatcgccggttgtaacgataagggagaagagaaagctcacgtcggtgaaccgcaggttaccgttcatattgtaaaaacggccccgttagaagttaagactgaattaccaggccgcaccaatgcttatcgtatagccgaagttcgcccacaggttagcgggatcgtactgaatcgcaatttcactgaaggcagcgatgtgcaagcaggccagtccctgtaccagatcgatcccgcgacctatcaggcaaattatgacagcgcgaaaggcgaactggcgaaaagtgaagccgccgccgccatcgcgcatttgacggtaaaacgttacgttccgctcgtgggtacgaaatacatcagccagcaggagtacgaccaggccattgctgatgctcgtcaggccgatgccgccgtgattgccgcaaaagccacagtcgaaagcgctcgcatcaatcttgcttataccaaagtcactgcgccaattagcggacgtatcggcaaatcgactgtgaccgaaggcgctcttgtcactaatgggcaaacgactgaactggcgactgtccagcagctcgatcctatctacgttgatgtgacccaatccagcaacgattttatgaggctgaagcaatccgtagagcaaggaaatttgcataaggaaaacgccaccagcaacgtagagttggtcatggaaaacggtcaaacctatcccctgaaaggtacgctgcaattctccgatgtgaccgttgatgaaagcaccggctccataaccctacgtgctgtcttccctaacccgcaacatacgcttttgccgggtatgtttgtgcgtgcacggattgatgaaggcgtccaacctgacgccattcttatcccgcaacaaggcgttagccgcacaccgcgtggtgatgcaaccgtgctgattgttaacgataaaagtcaggttgaagcgcgccctgtcgttgccagtcaggcgattggcgataaatggttgattagtgaaggactgaaatctggcgatcaagtcattgtcagcggcctgcaaaaagcgcgtccgggagagcaggttaaagccactaccgatacccccgcagatactgcatcgaagtaa

SEQ ID NO: 22 - protein, 385 aa, Escherichia coli
MTKHARFFLLPSFILISAALIAGCNDKGEEKAHVGEPQVTVHIVKTAPLEVKTELPGRTNAYRIAEVRPQVSGIVLNRNFTEGSDVQAGQSLYQIDPATYQANYDSAKGELAKSEAAAAIAHLTVKRYVPLVGTKYISQQEYDQAIADARQADAAVIAAKATVESARINLAYTKVTAPISGRIGKSTVTEGALVTNGQTTELATVQQLDPIYVDVTQSSNDFMRLKQSVEQGNLHKENATSNVELVMENGQTYPLKGTLQFSDVTVDESTGSITLRAVFPNPQHTLLPGMFVRARIDEGVQPDAILIPQQGVSRTPRGDATVLIVNDKSQVEARPVVASQAIGDKWLISEGLKSGDQVIVSGLQKARPGEQVKATTDTPADTASK

SEQ ID NO: 23 - DNA, 28 bp, synthetic sequence
gaaagtcgacctgccctacaccaacagc

SEQ ID NO: 24 - DNA, 29 bp, synthetic sequence
catggaattcgcatcaggggcagcccgtt

SEQ ID NO: 25 - DNA, 1155 bp, Pseudomonas aeruginosa
atggctgatttgcgtgcaataggaaggatcggggcggcgttggctatggccatcgcgttggcgggttgtgggccggcggaagagcgacaggaggccgccgaaatggtgttgccggtggaggtcctgacggtgcaggccgagcccctggcgctgagttcggaactgcctgggcggatcgaaccggtgcgggtcgccgaggtgcgcgcgcgggtggccggcatcgtcgtgcggaagcgcttcgaggagggcgccgacgtcaaggctggcgacctgctgttccagatcgatccggcaccgctgaaggctgcggtgtcgcgcgccgagggtgagctggcgcggaaccgcgcggtgctgttcgaggcgcaggcgcgggtgcgtcgctacgagccgctggtgaagatccaggcggtcagccagcaggacttcgataccgccaccgccgacctgcgcagcgccgaggcggcgacccgctcggcccaggccgacctggagaccgcgcgcctgaacctcggctacgcctcggtcactgcgccgatctccgggcgcatcggccgcgcgctggtgaccgagggcgcgctggtcgggcagggcgaggcgacgctgatggcgcgcatccagcagctcgatccgatctatgcggatttcacccagaccgcggccgaggccctgcgcctgcgcgacgccctgaagaaaggcaccttggccgccggcgacagccaggcgctgaccctgcgcgtcgaagggacgccctacgagcgccagggcgcgttgcagttcgccgacgtggcggtggatcgcggcaccggccagatcgccctgcgcggcaagttcgccaaccccgacggggtcctgctgccgggcatgtacgtgcgcgtacgtacgccccagggcatcgacaaccaggcgatcctggtgccgcaacgggccgtgcaccgctccagcgacggcagcgcccaggtgatggtggtgggcgccgacgagcgcgccgagtcgcgcagcgtcggtaccggcgtcatgcagggttcgcgctggcagatcaccgagggcctggagccgggtgaccgggtcatagtcggcggcctggctgcggtgcagccgggggtgaagatcgtgccgaagccggatggtgcccaggcgcaagcccagtcacctgcg

SEQ ID NO: 26 - protein, 384 aa, Pseudomonas aeruginosa
MADLRAIGRIGALAMAIALAGCGPAEERQEAAEMVLPVEVLTVQAEPLALSSELPGRIEPVRVAEVRARVAGIVVRKRFEEGADVKAGDLLFQIDPAPLKAAVSRAEGELARNRAVLFEAQARVRRYEPLVKIQAVSQQDFDTATADLRSAEAATRSAQADLETARLNLGYASVTAPISGRIGRALVTEGALVGQGEATLMARIQQLDPIYADFTQTAAEALRLRDALKKGTLAAGDSQALTLRVEGTPYERQGALQFADVAVDRGTGQIALRGKFANPDGVLLPGMYVRVRTPQGIDNQAILVPQRAVHRSSDGSAQVMVVGADERAESRSVGTGVMQGSRWQITEGLEPGDRVIVGGLAAVQPGVKIVPKPDGAQAQAQSPA

SEQ ID NO: 27 - DNA, 1158 bp, Salmonella typhimurium
atgacgaaacatgccaggttttcactcctgccctcattcatcatattctctgctgcgctgctggccggttgtaatgaccagggagatacccaggctcatgccggcgagccgcaagtcaccgtccatgtggtcgaaacagcgccgctagccgtaacgaccgaacttcccggacgtacgtccgcatttcgcattgcggaggttcgcccccaggtgagcgggatcgtgcttaaaagaaacttcaccgaaggtagcgatgtagaggccgggcagtcgctctatcagatcgatcctgccacttatcaggctgattatgacagcgctaaaggcgaacttgctaaaagcgaagcggctgcggctatcgcgcacctgacggtcaaacgctatgttccactggtcggcacaaaatatatcagccaacaggaatatgatcaggcgattgccgacgcccgccaggccgatgccgccgttgtggcggcaaaagccgctgttgaaagcgcgcgtattaaccttgcgtataccaaagtcacctcacccatcagcgggcgtataggaaaatctaatgtgactgaaggcgcgctggtgactaatggtcagtcaactgaactggctaccgtgcaacaactcgatccgatttatgtcgacgtgacgcaatcaagcaacgactttatgcgactcaagcaatccgtcgaacaaggtaacctgcataaagacagcgccagtagcacggttcaactggtaatggaaaatggtcaggtctacccgattaaaggcacgctgcaattttccgacgttaccgtagatgaaagcaccggctctatcacgctcagggcggtgttccctaacccgcaacacagtctgcttcccggtatgtttgttcgcgcccgcattgatgaaggcgtccagcccaatgccatccttgtcccccagcagggcgtaacccgcacgccgcgcggcgacgcaatggtgatggtggttaacgataaaagccaggtcgaagcccgcaatgtcgtggcggcgcaggctattggcgataaatggctcatcagcgaagggttaaaaccgggcgataaggtcatcgtcagcggcttacaaaaagcgcgaccgggcgtccaggtgaaagccactaccgatgctcctgcagcgaaaacggcgcaataa

SEQ ID NO: 28 - protein, 385 aa, Salmonella typhimurium
MTKHARFSLLPSFIIFSAALLAGCNDQGDTQAHAGEPQVTVHVVETAPLAVTTELPGRTSAFRIAEVRPQVSGIVLKRNFTEGSDVEAGQSLYQIDPATYQADYDSAKGELAKSEAAAAIAHLTVKRYVPLVGTKYISQQEYDQAIADARQADAAVVAAKAAVESARINLAYTKVTSPISGRIGKSNVTEGALVTNGQSTELATVQQLDPIYVDVTQSSNDFMRLKQSVEQGNLHKDSASSTVQLVMENGQVYPIKGTLQFSDVTVDESTGSITLRAVFPNPQHSLLPGMFVRARIDEGVQPNAILVPQQGVTRTPRGDAMVMVVNDKSQVEARNVVAAQAIGDKWLISEGLKPGDKVIVSGLQKARPGVQVKATTDAPAAKTAQ

SEQ ID NO: 29 - DNA, 3105 bp, Escherichia coli
atggcaaacttttttattcgacgaccgatatttgcatgggtgctggccattattctgatgatggcgggcgcactggcgatcctacaattgcccgtcgctcagtatccaacaattgcaccgcctgcggtttctgtttcagcaaactatccgggcgctgatgcgcagaccgtgcaggatacggtgacgcaggttatcgaacagaatatgaacggtatcgataacctgatgtatatgtcctccaccagcgattccgccggtagcgtgacaattacccttaccttccagtccgggaccgatcctgatatcgcgcaagtgcaggtgcagaacaaactccagctcgccacgccgttgctgccgcaggaggttcagcagcaggggatcagtgttgaaaagtccagtagcagctatttgatggtggcgggctttgtctctgataacccaggcaccacacaggacgatatctcggactatgtggcctctaacgttaaagatacgcttagccgtctgaatggcgtcggtgacgtacagcttttcggcgcacagtatgcgatgcgtatctggctggatgccgatctgctaaacaaatataaactgacaccggttgatgtgattaaccagttgaaggtacagaacgatcagatcgctgccggacagttgggcggaacgccagcgttaccagggcaacaattgaacgcctcgattattgctcagacgcggtttaaaaatccggaagaattcggcaaagtgaccctgcgcgtaaacagtgacggctcggtggtacgcctgaaagatgtcgcacgggttgaacttggcggtgaaaactataacgttatcgctcgtatcaacggaaaaccggcggcgggcctggggattaagctggcaaccggcgcgaatgctctcgataccgcgaaagccattaaggcaaaactggcggaattacagccattcttcccgcagggaatgaaggttctctacccttatgacaccacgccattcgtccagctttctattcacgaagtggtaaaaacgctgttcgaagccattatgctggtgttcctggtgatgtatctgttcttgcagaatatgcgagcaacgctgatccccaccattgcggtacccgtggtgttgttagggacgtttgccatcctcgccgcttttggttactccatcaacacactaacgatgttcgggatggtgcttgccatcgggctgctcgtcgatgatgcgatagtggtggtggagaacgtcgagcgcgtgatgatggaggataagctcccgccaaaagaagcgacggaaaaatcgatgtcgcaaattcagggcgcactggtgggtatcgcgatggtgctgtcagcggtatttattccgatggcattcttcggcggttctactggggcaatttatcgccagttctctatcaccatcgtttcggcaatggcgctttctgttctggtggcattgattcttacccctgcgttatgtgcaacgctgcttaaacccgtctctgctgagcatcacgaaaataagggcggtttcttcggttggtttaataccaccttcgatcatagcgttaaccactacaccaacagcgtcggcaaaatcctcggatccacaggacgatatttactgatctatgcgctgattgttgcaggaatggtggtgttgtttttacgtcttccgtcttccttcttacctgaagaggatcagggtgtctttctgaccatgattcagttacccgctggcgcgacgcaagagcggacgcaaaaagtgttggatcaagttacggattactatctgaagaacgagaaagcgaacgttgaaagtgtctttacggttaacggctttagcttcagcggccaggcacaaaacgccggtatggccttcgtcagtctgaaaccgtgggaagagcgtaatggtgacgaaaacagtgcggaagcggtaatccatcgtgccaaaatggaattgggcaagatccgcgacggttttgtcattccattcaatatgccagccattgttgaactgggcacggcaacgggtttcgactttgagttaattgatcaggctgggctgggtcacgatgccctaacccaggcccgtaaccagttgcttggtatggcggcgcaacatcctgccagcttagtcagcgtgcgccctaatggcctggaagacaccgcgcagtttaaactggaagttgaccaggaaaaggcgcaggcattaggtgtttcactttctgacatcaatcagaccatttcaacggcgctgggtgggacttacgttaacgacttcatcgaccgtggccgcgtgaaaaagttgtatgttcaggcggatgccaaattccgtatgctgccagaagatgtcgataaactttatgtccgcagcgccaacggcgaaatggtgccattctcggcctttaccacttcacattgggtgtatggctctccgcgactggaacgctacaacggtctgccgtcaatggagattcagggggaagccgcgccaggaaccagttccggcgatgccatggcgttgatggaaaaccttgcgtcaaaattacctgcgggcattggttatgactggacgggtatgtcgtatcaggaacgcttatcgggaaaccaggctcccgctctggtagcaatttcctttgtggttgttttcctgtgccttgctgcactctatgaaagctggtcaattcctgtctcggttatgttggtagtgccgttagggattgtcggcgtgctgctggcggcgacactctttaatcaaaaaaatgacgtctactttatggtgggcttgctaacgacaattggcttgtcggccaaaaacgctattttgatcgttgagttcgctaaagatctcatggagaaagagggtaaaggtgttgttgaagcgacactgatggcagtacgtatgcgtctgcgtcctatcctgatgacctctctcgcctttattctcggcgtattaccgctagctatcagtaacggtgccggcagtggcgcgcagaacgctgtgggtatcggggtaatgggaggaatggtctctgcaacgttgctggcaatcttcttcgtaccggtgttctttgtggtgatccgccgttgctttaaaggataa

SEQ ID NO: 30 - protein, 1034 aa, Escherichia coli
MANFFIRRPIFAWVLAIILMMAGALAILQLPVAQYPTIAPPAVSVSANYPGADAQTVQDTVTQVIEQNMNGIDNLMYMSSTSDSAGSVTITLTFQSGTDPDIAQVQVQNKLQLATPLLPQEVQQQGISVEKSSSSYLMVAGFVSDNPGTTQDDISDYVASNVKDTLSRLNGVGDVQLFGAQYAMRIWLDADLLNKYKLTPVDVINQLKVQNDQIAAGQLGGTPALPGQQLNASIIAQTRFKNPEEFGKVTLRVNSDGSVVRLKDVARVELGGENYNVIARINGKPAAGLGIKLATGANALDTAKAIKAKLAELQPFFPQGMKVLYPYDTTPFVQLSIHEVVKTLFEAIMLVFLVMYLFLQNMRATLIPTIAVPVVLLGTFAILAAFGYSINTLTMFGMVLAIGLLVDDAIVVVENVERVMMEDKLPPKEATEKSMSQIQGALVGIAMVLSAVFIPMAFFGGSTGAIYRQFSITIVSAMALSVLVALILTPALCATLLKPVSAEHHENKGGFFGWFNTTFDHSVNHYTNSVGKILGSTGRYLLIYALIVAGMVVLFLRLPSSFLPEEDQGVFLTMIQLPAGATQERTQKVLDQVTDYYLKNEKANVESVFTVNGFSFSGQAQNAGMAFVSLKPWEERNGDENSAEAVIHRAKMELGKIRDGFVIPFNMPAIVELGTATGFDFELIDQAGLGHDALTQARNQLLGMAAQHPASLVSVRPNGLEDTAQFKLEVDQEKAQALGVSLSDINQTISTALGGTYVNDFIDRGRVKKLYVQADAKFRMLPEDVDKLYVRSANGEMVPFSAFTTSHWVYGSPRLERYNGLPSMEIQGEAAPGTSSGDAMALMENLASKLPAGIGYDWTGMSYQERLSGNQAPALVAISFVVVFLCLAALYESWSIPVSVMLVVPLGIVGVLLAATLFNQKNDVYFMVGLLTTIGLSAKNAILIVEFAKDLMEKEGKGVVEATLMAVRMRLRPILMTSLAFILGVLPLAISNGAGSGAQNAVGIGVMGGMVSATLLAIFFVPVFFVVIRRCFKG

SEQ ID NO: 31 - DNA, 28 bp, synthetic sequence
acttgaattctgaccgacggccacgagt

SEQ ID NO: 32 - DNA, 29 bp, synthetic sequence
attaggatccacacgcacgacgaccactg

SEQ ID NO: 33 - DNA, 3132 bp, Pseudomonas aeruginosa
atgtccgaattcttcatcaagcggccgaacttcgcctgggtggtggccctgttcatctccctggccggcctgctggtcatttccaaattgccggtagcgcagtaccccaatgtcgcgccgccacagatcaccatcaccgccacctatcccggcgcctcggcgaaggtgctggtggactccgtcaccagtgtgctcgaggagtcgctgaacggcgccaagggcctgctctacttcgagtcgaccaacaactccaacggcaccgccgagatcgtcgtcaccttcgagccgggcaccgatccggacctggcccaggtggacgtgcagaaccgcctgaagaaagccgaggcgcgcatgccgcaggcggtgctgacccagggcctgcaggtcgagcagaccagcgccggtttcctgctgatctatgcgctcagctacaaggaaggcgctcagcgcagcgacaccaccgccctcggcgactacgccgcgcgcaatatcaacaacgagctgcggcgcctgccgggcgtcggcaagctgcaattcttctcttccgaggcggccatgcgggtctggatcgatccgcagaagctggtgggcttcggcctctccatcgacgacgtgagcaatgccatccgcgggcagaacgtgcaggtgccggccggcgccttcggcagcgcaccgggcagttccgcgcaggagctgacggcgaccctggcggtgaagggcaccctggacgatccgcaggagttcggccaggtagtgctgcgcgccaacgaggacggctcgctggtccggctcgccgatgtcgcgcgcctggaactcggcaaggagagctacaacatttcctcgcgactgaacggcacgcccaccgtgggcggggctatccagctgtcgcccggggccaacgcgatccagaccgctaccctggtgaaacagcgtctcgccgaactgtcggcgttcttccccgaggacatgcagtacagcgtgccctacgacacctcgcgcttcgtcgacgtggccatcgagaaggtgatccacaccctgatcgaagcgatggtcctggtgttcctggtgatgttcctgttcctgcagaacgtccgctacaccctgatcccgtccatcgtggtgccggtgtgcctgctgggtacgctgatggtgatgtacctgctggggttctcggtgaacatgatgaccatgttcggcatggtcctggcgatcggcatcctggtggacgacgccatcgtggtggtggagaacgtcgagcggatcatggcggaggaggggatttccccggccgaggccacggtcaaggcgatgaagcaggtatccggcgccatcgtcggcatcaccctggtgctctcggcggtgttcctgccgctggctttcatggccggttcggtgggggtgatctaccagcagttctcggtgtcgctggcggtctcgatcctgttctccggcttcctcgccctgaccttcaccccggcgctgtgcgccacgctgctcaagcccattcccgaagggcaccacgagaagcgcggcttcttcggcgccttcaaccgtggcttcgcccgcgtcaccgagcgctattcgctgctcaactcgaagctggtggcgcgcgccggacgcttcatgctggtgtacgccggcctggtggccatgctcggctacttctacctgcgcctgccggaagccttcgtgccggcggaagacctcggctacatggtggtcgacgtgcaactgccgcctggcgcttcgcgcgtgcgcaccgatgccaccggcgaggagctcgagcgcttcctcaagtcccgcgaggcggtggcttcggtgttcctgatctcgggcttcagcttctccggccagggcgacaatgccgcgctggccttcccaaccttcaaggactggtccgagcgaggcgccgagcagtcggccgccgccgagatcgccgcgctgaacgagcatttcgcgctgcccgacgatggcacggtcatggccgtgtcgccgccaccgatcaacggtctgggtaactccggcggcttcgcattgcgcctgatggaccgtagcggggtcggccgcgaagcgctgctgcaggctcgcgatactcttcttggcgagatccagaccaacccgaaattcctttacgcgatgatggaaggactggccgaagcgccgcaactgcgcctgttgatcgaccgggagaaggcccgtgccctgggggtgagcttcgagaccatcagcggcacgctgtccgctgccttcggctcggaggtgatcaacgacttcaccaatgcggggcgccaacagcgggtggtgatccaggccgaacagggcaaccggatgaccccggaaagcgtgctcgagctatacgtgcctaacgctgctggcaacctggtaccgctcagcgccttcgtcagcgtgaaatgggaagagggaccggtgcaattggtgcgctataacggctacccgtcgatccgcatcgtcggtgacgccgcgcccggcttcagtaccggcgaagccatggcggaaatggagcgcctggcctcgcagctgccggccggcatcggctacgagtggaccggcctgtcctatcaggagaaggtctccgccgggcaggccaccagcctgttcgccctcgccatcctggtggtgttcctgttgctggtggcgctctacgagagctggtcgatcccgctgtcggtgatgctgatcgtgccgatcggcgccatcggcgcggtgctcgcggtgatggtcagcggtatgtccaacgacgtgtatttcaaggtcggcctgatcaccatcatcggtctttcggcgaagaacgcgatcctcatcgtcgagttcgccaaggaactctgggagcaggggcatagcctgcgcgacgccgccatcgaggccgcgcgcctgcgcttccggccgatcatcatgacttccatggcgttcatcctcggcgtgatacccctggccctggccagcggtgccggcgcggcgagccagcgtgccatcggcaccggagtgatcggcgggatgctcagcgccaccttcctcggcgtgctgttcgtacctatctgtttcgtctggctgctgtcgctgctgcgcagcaagccggcacccatcgaacaggccgcttcggccggggagtga

SEQ ID NO: 34 - protein, 1043 aa, Pseudomonas aeruginosa
MSEFFIKRPNFAWVVALFISLAGLLVISKLPVAQYPNVAPPQITITATYPGASAKVLVDSVTSVLEESLNGAKGLLYFESTNNSNGTAEIVVTFEPGTDPDLAQVDVQNRLKKAEARMPQAVLTQGLQVEQTSAGFLLIYALSYKEGAQRSDTTALGDYAARNINNELRRLPGVGKLQFFSSEAAMRVWIDPQKLVGFGLSIDDVSNAIRGQNVQVPAGAFGSAPGSSAQELTATLAVKGTLDDPQEFGQVVLRANEDGSLVRLADVARLELGKESYNISSRLNGTPTVGGAIQLSPGANAIQTATLVKQRLAELSAFFPEDMQYSVPYDTSRFVDVAIEKVIHTLIEAMVLVFLVMFLFLQNVRYTLIPSIVVPVCLLGTLMVMYLLGFSVNMMTMFGMVLAIGILVDDAIVVVENVERIMAEEGISPAEATVKAMKQVSGAIVGITLVLSAVFLPLAFMAGSVGVIYQQFSVSLAVSILFSGFLALTFTPALCATLLKPIPEGHHEKRGFFGAFNRGFARVTERYSLLNSKLVARAGRFMLVYAGLVAMLGYFYLRLPEAFVPAEDLGYMVVDVQLPPGASRVRTDATGEELERFLKSREAVASVFLISGFSFSGQGDNAALAFPTFKDWSERGAEQSAAAEIAALNEHFALPDDGTVMAVSPPPINGLGNSGGFALRLMDRSGVGREALLQARDTLLGEIQTNPKFLYAMMEGLAEAPQLRLLIDREKARALGVSFETISGTLSAAFGSEVINDFTNAGRQQRVVIQAEQGNRMTPESVLELYVPNAAGNLVPLSAFVSVKWEEGPVQLVRYNGYPSIRIVGDAAPGFSTGEAMAEMERLASQLPAGIGYEWTGLSYQEKVSAGQATSLFALAILVVFLLLVALYESWSIPLSVMLIVPIGAIGAVLAVMVSGMSNDVYFKVGLITIIGLSAKNAILIVEFAKELWEQGHSLRDAAIEAARLRFRPIIMTSMAFILGVIPLALASGAGAASQRAIGTGVIGGMLSATFLGVLFVPICFVWLLSLLRSKPAPIEQAASAGE

SEQ ID NO: 35 - DNA, 3114 bp, Salmonella typhimurium
atggcaaacttttttattagacgtcctattttcgcctgggttctggccattatcctgatgatggctggcgcactggcaataatgcaacttcccgttgcgcagtatccaaccattgcgccgccagcggtttctatttctgcaacctatcctggcgcggatgcgcagacggtacaggatacggttactcaggttatcgaacaaaatatgaacggtatcgataacctgatgtatatgtcctctaccagcgactctgctggtagcgtgaccatcaccctgaccttccagtccggaaccgatccggatatcgcgcaggttcaggtgcaaaataaattgcagctcgccacgcctttactgccgcaagaagtccagcagcaggggattagcgttgaaaaatccagcagcagctttttgatggtcgccgggttcgtctcagataatccgaacactacccaggacgacatctctgactatgtcgcctctaacattaaggattctatcagccgtctgaatggtgtgggcgacgttcagctatttggcgcacagtacgccatgcgtatctggctggatgcgaatctgctaaataaataccagctcacgccagttgacgtcatcaaccagttaaaagtacagaacgaccagattgcggcaggccaactgggcggcacgccagcattaccgggccagcagcttaacgcctcaatcatcgcccaaacgcgtctgaaagatccggaagagttcggcaaagttacgttgcgcgtcaataccgacggctctgtcgtccatctcaaagatgtcgcgcgtattgagcttggcggtgaaaactataacgttgtagcgcgcattaacggtaaaccggcctccggtctcggtattaaactggcgaccggcgctaacgcgctggataccgcaaccgcaatcaaagtgaaactggcggagctgcagcctttcttccctcagggaatgaaggtggtttatccttatgacacaacgcccttcgtaaaaatatctatccacgaagtggtaaaaacgctgtttgaagcgattattctggtgttcctggtaatgtatctgttcttacagaatatccgggcaaccctgattcctaccatcgctgttcctgtcgtgttgctaggcacttttgcggtactcgccgcctttggctattccatcaataccctgacgatgtttggtatggtactggcgatagggctgttggttgacgatgcgatagtggtcgtagaaaacgttgaacgtgtaatgatggaggataacctttctccccgagaggcgacggaaaaatccatgtcgcagattcagggagcgctggttggtatcgcgatggtactgtctgcggtatttatcccgatggccttttttggcggctcgaccggggcaatttatcgccagttctctattactattgtttcagcaatggcgctatccgttctggttgcgttgattctgacgccagcactgtgcgctacgctgcttaaacccgtatctgctgaacatcacgagaaaaaaagcggcttctttggctggttcaataccaggtttgaccacagcgttaaccactatactaacagcgtaagcggcatcgtgcgtaatacgggtcgctatctcattatctatctacttattgtagtcggaatggcggttctgtttttacgcctcccgacctccttcctgccggaagaagatcagggagtattcctgaccatgattcagctcccctctggcgctacgcaagaacgtacgcagaaagtgctggatcaagtcactcattactacctgaataatgaaaaagcgaacgtcgaaagcgtgtttaccgtaaacggctttagctttagcggtcagggacaaaactcagggatggcatttgtcagccttaaaccctgggaagagcgtaacggtgaagaaaatagcgtcgaagccgttatcgccagagcgacacgcgcctttagccagattcgcgacgggttggtgttccccttcaacatgccggcaattgtcgagttaggtaccgcaacaggtttcgacttcgaactgattgatcagggaggactcggtcacgatgcgttaacaaaagcgcgtaatcaactcctgggtatggtcgcgaagcatcctgatctattagtgcgcgtacgcccgaacgggctggaagacacgccacagttcaagctggatgtcgatcaagaaaaagcgcaggcgctcggcgtttcgctgtctgatatcaacgaaaccatctccgcggcgttgggcggctattacgttaacgactttatcgatcgcggacgagtgaaaaaagtatacgttcaggctgacgctcagttccgtatgctgccgggagatatcaacaatctttatgttcgcagcgctaatggcgagatggtgcccttctctacctttagctcagcacggtggatttatggttcgccacgcctggaacgctataacgggatgccgtcaatggaactgctcggcgaagcagcacccggacgaagcaccggtgaagccatgtcgttaatggaaaacctggcttcacagctaccaaacggtattggctatgactggacaggtatgtcgtatcaggaacggttgtcaggtaaccaggcgccggcgctgtacgcaatctcactcattgtcgttttcctctgccttgccgctctgtatgaaagctggtcaattccgttctcggtaatgctggtcgtaccgctcggcgtggttggcgctctgcttgcagcgtcattgcgcggtctgaacaatgacgtttacttccaggttggcttgttaaccactattggcctttctgctaaaaacgccatcctgattgtcgagttcgccaaagatctcatggaaaaagaaggacgtggattgattgaagcgacgctggaagcatcccgtatgcgtttacgtcctattctaatgacctcgctggcctttattctcggggtaatgccgttagttatcagtcgtggcgcaggtagtggtgcacagaacgcagtaggcacaggggttatggggggaatgttaaccgcaaccttattagctatcttcttcgtgccggtattcttcgttgttgtaaaacgccgatttaatcgccatcatgattaa

SEQ ID NO: 36 - protein, 1037 aa, Salmonella typhimurium
MANFFIRRPIFAWVLAIILMMAGALAIMQLPVAQYPTIAPPAVSISATYPGADAQTVQDTVTQVIEQNMNGIDNLMYMSSTSDSAGSVTITLTFQSGTDPDIAQVQVQNKLQLATPLLPQEVQQQGISVEKSSSSFLMVAGFVSDNPNTTQDDISDYVASNIKDSISRLNGVGDVQLFGAQYAMRIWLDANLLNKYQLTPVDVINQLKVQNDQIAAGQLGGTPALPGQQLNASIIAQTRLKDPEEFGKVTLRVNTDGSVVHLKDVARIELGGENYNVVARINGKPASGLGIKLATGANALDTATAIKVKLAELQPFFPQGMKVVYPYDTTPFVKISIHEVVKTLFEAIILVFLVMYLFLQNIRATLIPTIAVPVVLLGTFAVLAAFGYSINTLTMFGMVLAIGLLVDDAIVVVENVERVMMEDNLSPREATEKSMSQIQGALVGIAMVLSAVFIPMAFFGGSTGAIYRQFSITIVSAMALSVLVALILTPALCATLLKPVSAEHHEKKSGFFGWFNTRFDHSVNHYTNSVSGIVRNTGRYLIIYLLIVVGMAVLFLRLPTSFLPEEDQGVFLTMIQLPSGATQERTQKVLDQVTHYYLNNEKANVESVFTVNGFSFSGQGQNSGMAFVSLKPWEERNGEENSVEAVIARATRAFSQIRDGLVFPFNMPAIVELGTATGFDFELIDQGGLGHDALTKARNQLLGMVAKHPDLLVRVRPNGLEDTPQFKLDVDQEKAQALGVSLSDINETISAALGGYYVNDFIDRGRVKKVYVQADAQFRMLPGDINNLYVRSANGEMVPFSTFSSARWIYGSPRLERYNGMPSMELLGEAAPGRSTGEAMSLMENLASQLPNGIGYDWTGMSYQERLSGNQAPALYAISLIVVFLCLAALYESWSIPFSVMLVVPLGVVGALLAASLRGLNNDVYFQVGLLTTIGLSAKNAILIVEFAKDLMEKEGRGLIEATLEASRMRLRPILMTSLAFILGVMPLVISRGAGSGAQNAVGTGVMGGMLTATLLAIFFVPVFFVVVKRRFNRHHD

SEQ ID NO: 37 - DNA, 333 bp, Escherichia coli
atgaacccttatatttatcttggtggtgcaatacttgcagaggtcattggtacaaccttaatgaagttttcagaaggttttacacggttatggccatctgttggtacaattatttgttattgtgcatcattctggttattagctcagacgctggcttatattcctacagggattgcttatgctatctggtcaggagtcggtattgtcctgattagcttactgtcatggggatttttcggccaacggctggacctgccagccattataggcatgatgttgatttgtgccggtgtgttgattattaatttattgtcacgaagcacaccacattaa

SEQ ID NO: 38 - protein, 110 aa, Escherichia coli
MNPYIYLGGAILAEVIGTTLMKFSEGFTRLWPSVGTIICYCASFWLLAQTLAYIPTGIAYAIWSGVGIVLISLLSWGFFGQRLDLPAIIGMMLICAGVLIINLLSRSTPH

SEQ ID NO: 39 - DNA, 27 bp, synthetic sequence
cttggtcgacaagacgccgtcataatc

SEQ ID NO: 40 - DNA, 29 bp, synthetic sequence
actggaattcacgaagcacaccacattaa

SEQ ID NO: 41 - DNA, 330 bp, Pseudomonas aeruginosa
atgaccaactatctctacctcgccatcgccatcgccgccgaagtggtcgccaccacctcgctgaaagccgtcgccggattcagcaagccactgccgctgctgctggtggtgggcggctacgtgctcgccttcagcatgctcgtgctggtcatgcgcaccctgccggtcggcgtggtctacgccatctggtccggactcggcatcgtcctggtcagcctggtggcgatgttcgtctacggccagcgcctggaccccgccgccctcctcggcatcggcctgatcatcgccggcgtgctggtgatccagttgttctcccgcgcttcggggcac

SEQ ID NO: 42 - protein, 110 aa, Pseudomonas aeruginosa
MTNYLYLAIAIAAEVVATTSLKAVAGFSKPLPLLLVVGGYVLAFSMLVLVMRTLPVGVVYAIWSGLGIVLVSLVAMFVYGQRLDPAALLGIGLIIAGVLVIQLFSRASGH

SEQ ID NO: 43 - DNA, 336 bp, Salmonella typhimurium
atgactaaagaagctgtaatctttctatttatcgccatcgtggtagaagttatcgccacgatctcattaaaattatcggatagttttacgcgtctggtaccgagcctcgttaccatcatcggatattgtatcgcgttctggtgccttaccatccctatgcgaaccatccctgcgggtatcatttatgccatttggtctggggtagggattgttcttattggattgataggatggctgtttcttggccaaaaattggatgtgccggctattattggcatgttgcttatcatctgcggtgtaatcgtaatcaatctgttttcaaaaagcgtcagtcac

SEQ ID NO: 44 - protein, 112 aa, Salmonella typhimurium
MTKEAVIFLFIAIVVEVIATISLKLSDSFTRLVPSLVTIIGYCIAFWCLTIPMRTIPAGIIYAIWSGVGIVLIGLIGWLFLGQKLDVPAIIGMLLIICGVIVINLFSKSVSH

SEQ ID NO: 45 - DNA, 1182 bp, Escherichia coli
atgaaaaggcaaagaaacgtcaatttgttattgatgttggtattactcgtggccgtcggtcagatggcgcaaaccatttatattccagctattgccgatatggcgcgcgatctcaacgtccgtgaaggggcggtgcagagcgtaatgggcgcttatctgctgacttacggtgtctcacagctgttttatggcccgatttccgaccgcgtgggccgccgaccggtgatcctcgtcggaatgtccatttttatgctggcaacgctggtcgcggtcacgacctccagtttgacggtgttgattgccgccagcgcgatgcaggggatgggcaccggcgttggcggcgtaatggcgcgtactttaccgcgagatttatatgaacggacacagttgcgccatgctaacagcctgttaaacatggggattctcgtcagtccgttgctcgcaccgctaatcggcggtctgctggatacgatgtggaactggcgcgcctgttatctctttttgttggttctttgtgctggtgtgaccttcagtatggcccgctggatgccggaaacgcgtccggtcgatgcaccgcgcacgcgcctgcttaccagttataaaacgcttttcggtaacagcggttttaactgttatttgctgatgctgattggcggtctggccgggattgccgcctttgaagcctgctccggcgtgctgatgggcgcggtgttagggctgagcagtatgacggtcagtattttgtttattctgccgattccggcagcgttttttggcgcatggtttgccggacgtcccaataaacgcttctccacgttaatgtggcagtcggttatctgctgcctgctggctggcttgctgatgtggatccccgactggtttggcgtgatgaatgtctggacgctgctcgttcccgccgcgctgttctttttcggtgccgggatgctgtttccgctggcgaccagcggcgcgatggagccgttccccttcctggcgggcacggctggcgcgctggtcggcggtctgcaaaacattggttccggcgtgctggcgtcgctctctgcgatgttgccgcaaaccggtcagggcagcctggggttgttgatgaccttaatgggattgttgatcgtgctgtgctggctgccgctggcgacgcggatgtcgcatcaggggcagcccgtt

SEQ ID NO: 46 - protein, 394 aa, Escherichia coli
MKRQRNVNLLLMLVLLVAVGQMAQTIYIPAIADMARDLNVREGAVQSVMGAYLLTYGVSQLFYGPISDRVGRRPVILVGMSIFMLATLVAVTTSSLTVLIAASAMQGMGTGVGGVMARTLPRDLYERTQLRHANSLLNMGILVSPLLAPLIGGLLDTMWNWRACYLFLLVLCAGVTFSMARWMPETRPVDAPRTRLLTSYKTLFGNSGFNCYLLMLIGGLAGIAAFEACSGVLMGAVLGLSSMTVSILFILPIPAAFFGAWFAGRPNKRFSTLMWQSVICCLLAGLLMWIPDWFGVMNVWTLLVPAALFFFGAGMLFPLATSGAMEPFPFLAGTAGALVGGLQNIGSGVLASLSAMLPQTGQGSLGLLMTLMGLLIVLCWLPLATRMSHQGQPV

SEQ ID NO: 47 - DNA, 30 bp, synthetic sequence
acttgaattcattaaggttgtaccaatgac

SEQ ID NO: 48 - DNA, 29 bp, synthetic sequence
aattggatccgccaacactgcgacagcgt

SEQ ID NO: 49 - DNA, 1176 bp, Pseudomonas aeruginosa
atgaacctgcgcatcctgctgatactgggagccctgagcgccttcgggccactggccatcgatttctacctgccgagcttcccgaccctggccaaggccttcgccaccgacgtcgagcacgtccagctgagcctggccgcctacttcgccggcctgtccatcggccagttgctctacggcccggtggccgaccgctatggccggcgcctgccgctgctggtgggggtgagtatcttcaccctggcctcggccgcctgcgccctggcgccgagcctggagtggctgatcgccgcgcgcttcgtccaggccctcggcggctgcgccgggatggtcatctcgcgggcagtggtgcgtgacctgtgcgatccgatcggcgccgccaaggcgttttcccagttgatgctggtgatgggcctggcgccgatcctcgcccccgtggccggcggcctgctgctggactggcagggctggcaatcgatcttcgtctgcctgaccctgttcgccgccctggccggcctcggcgtggcgttgtggctgccggaaaccatcccggccgacggcgagcgaccgccgctgtccggcgcgctggggcagtaccggcggctgttcgccgaccgcctgtttctcgcctatggcctttccggcggggtggcgatggccgggatgttcgcctacatcgccggttcgccctacgtgttcatccagctctacgggattcccgccgagcactatggctggctgttcggcagcaatgccgcgggcttcatcgtcatggcacaggtcaacgcgcggctgctgcgctatcgcgggccggcgttctggctgcggcggatcatctggggctacctggcgtgcggcctggccctgctgggcatcgcttcgctgcaaccggccacgctgtggccgctgctggttccgctgttcggatgcgtggccagcctcggctgcatcattcccaacgcttccgcctgcgccatggccggacagggacagcacgccggcagcgcctcggcgctgatgggcagcctgcagttcagcgtggcggcgggcgcttcggcgctggtcggcgtgctccacgacggcacggcgatccccatgactctggtgatcgccgtctgcggcgcggtcgcgaccctgctcgcctggcagaccgggcgcctggagcgccaggccgcc

SEQ ID NO: 50 - protein, 392 aa, Pseudomonas aeruginosa
MNLRILLILGALSAFGPLAIDFYLPSFPTLAKAFATDVEHVQLSLAAYFAGLSIGQLLYGPVADRYGRRLPLLVGVSIFTLASAACALAPSLEWLIAARFVQALGGCAGMVISRAVVRDLCDPIGAAKAFSQLMLVMGLAPILAPVAGGLLLDWQGWQSIFVCLTLFAALAGLGVALWLPETIPADGERPPLSGALGQYRRLFADRLFLAYGLSGGVAMAGMFAYIAGSPYVFIQLYGIPAEHYGWLFGSNAAGFIVMAQVNARLLRYRGPAFWLRRIIWGYLACGLALLGIASLQPATLWPLLVPLFGCVASLGCIIPNASACAMAGQGQHAGSASALMGSLQFSVAAGASALVGVLHDGTAIPMTLVIAVCGAVATLLAWQTGRLERQAA

SEQ ID NO: 51 - DNA, 1182 bp, Salmonella typhimurium
atgaaaaggcagagaaacgtcaatttgttgttgatgttggtgttactggtggccgtagggcagatggcgcaaaccatttatattcccgccatcgccgatatggcacaagcgctaaacgtccgggaaggcgccgtccagagcgtaatggctgcttacctcctgacctacggcgtctcgcaactgttttacggcccgctttccgaccgggttgggcgccgcccggtaatcctcgtcggcatgtctatttttatggtagcgactctgatcgccatgaccacgcatagtttgacggtgttgattgccgccagcgccatgcaagggatgggaaccggcgttggcggagtaatggcgagaacgctcccgcgcgatctgtatgaaggaacgcaacttcgtcatgccaatagcctgttaaatatggggattctggtcagcccgctgttagcgccgctgattggcggcctgctggataccctgtggaactggcgcgcgtgttacgctttcctgctggtgctttgcgctggcgtcaccttcagcatggcgcgctggatgccggaaacccgccccgccggcgcgccgcgtacgcggctgatcgccagctataagacgctgtttggcaacggcgcatttaactgttacctgctgatgctaatcggcgggctggctggcattgcggtctttgaagcctgttccggcgtgctgatgggagcagtattaggtctcagcagtatggtggtaagcattctgtttattctgccgattccggcggcgttcttcggcgcctggtttgccggacgcccgaataaacgcttctcagccctgatgtggcagtcagttatttgctgtctgctggcaggccttatgatgtggattcccggctggtttggcgtgatgaacgtctggacgctactcatccccgctgcgctgtttttcttcggcgccgggatgttatttccactggccaccagcggcgcgatggagccgtttccgttcctcgcaggcaccgctggcgcgctggtcggcgggctgcaaaatattggttccggcgtactggcgtggctttcggcaatgctgccgcaaaccggtcagggcagtctgggattgctgatgacccttatgggattgctgatcttggcgtgctggctgccgctggcgtcacggatatcgcatcagggacagacggtt

SEQ ID NO: 52 - protein, 394 aa, Salmonella typhimurium
MKRQRNVNLLLMLVLLVAVGQMAQTIYIPAIADMAQALNVREGAVQSVMAAYLLTYGVSQLFYGPLSDRVGRRPVILVGMSIFMVATLIAMTTHSLTVLIAASAMQGMGTGVGGVMARTLPRDLYEGTQLRHANSLLNMGILVSPLLAPLIGGLLDTLWNWRACYAFLLVLCAGVTFSMARWMPETRPAGAPRTRLIASYKTLFGNGAFNCYLLMLIGGLAGIAVFEACSGVLMGAVLGLSSMVVSILFILPIPAAFFGAWFAGRPNKRFSALMWQSVICCLLAGLMMWIPGWFGVMNVWTLLIPAALFFFGAGMLFPLATSGAMEPFPFLAGTAGALVGGLQNIGSGVLAWLSAMLPQTGQGSLGLLMTLMGLLILACWLPLASRISHQGQTV

SEQ ID NO: 53 - DNA, 795 bp, Escherichia coli
atggttgaattaaaagagccgcttgccacactttggcgtggtaaagatgcttttgcagaggtcaaaaaactgaacggcgaggtctttcgtgaactggagactcgtcgtacattacgctttgaactgtccgggaaaagctattttcttaaatggcacaaggggacgacattaaaagagattataaaaaatctactctcattgcggatgcccgttttgggcgcagacagagagtggcacgctattcatcgcctgagtgatgttggcgttgatacaatgaagggcattgggtttggcgaaaaagggttaaatccattaactcgcgcatcatttattattaccgaagatctcactcccacaattagccttgaagattattgtgccgattgggcagtcaacccgcctgatatacgtgttaagcgtatgctgatcgcacgtgtagcaactatggtgcgtaaaatgcatactgcagggataaatcaccgcgactgttacatttgccactttttgcttcatttgccatttactggccgggaagatgaattaaaaatttcagttatcgatctgcatcgggcacagatacgtgcaaaagtaccgcgccgctggcgcgataaagacctgattggtttatatttctcatcaatgaatattggcctgacgcaaagagatatctggcgatttatgaaggtttattttgggatgcctttacgcaaaatattgtctcttgaacagaatttattgaacatggcaagcgttaaggccgagcgtattaaagagcgaacacaacgcaaaggatta

SEQ ID NO: 54 - protein, 265 aa, Escherichia coli
MVELKEPLATLWRGKDAFAEVKKLNGEVFRELETRRTLRFELSGKSYFLKWHKGTTLKEIIKNLLSLRMPVLGADREWHAIHRLSDVGVDTMKGIGFGEKGLNPLTRASFIITEDLTPTISLEDYCADWAVNPPDIRVKRMLIARVATMVRKMHTAGINHRDCYICHFLLHLPFTGREDELKISVIDLHRAQIRAKVPRRWRDKDLIGLYFSSMNIGLTQRDIWRFMKVYFGMPLRKILSLEQNLLNMASVKAERIKERTQRKGL

SEQ ID NO: 55 - DNA, 807 bp, Pseudomonas aeruginosa
atgaggctggtgctggaagagccgttcaagcgcctgtggaacgggcgcgacccgttcgaggcggtggaggcgctgcaaggcaaggtctaccgcgaactggaagggcgccgcaccctgcgcaccgaggtcgacgggcgtggctacttcgtcaagatccaccgtggcatcggctggggcgagatcgccaagaacctgctcaccgccaagctcccggtgctcggcgcgcgccaggagtggcaggccatccggcgcctgcacgaggccggcgtagcgaccatgaccgcggtcgcctacggcgagcgcggcagcgatccggcgcggcagcattccttcatcgtcaccgaggaactggcgccgaccgtggacctcgaggtgttctcccaggactggcgcgaacgtcctccaccgccgcggctcaagcgcgcgctggtcgaggcggtggcgcggatggtcggcgacatgcaccgtgccggagtcaaccatcgcgactgctacatctgtcatttcctgttgcacaccgacaagccggtgagcgcggacgatttccgcctctcggtgatcgatctgcaccgtgcccagacccgcgacgccacgccgaaacgctggcgtaacaaggatctggcggcattgtatttctctgcgctggacatcggactgacgcgtcgcgacaagctacgcttcctgcgcacctatttccgccggccgttgcgcgagatactgcgcgacgaggccggcctgctggcctggatggaacgcaaggcggaaaaactctacgaacgcaagcagcgttacggagacctgctctga

SEQ ID NO: 56 - protein, 268 aa, Pseudomonas aeruginosa
MRLVLEEPFKRLWNGRDPFEAVEALQGKVYRELEGRRTLRTEVDGRGYFVKIHRGIGWGEIAKNLLTAKLPVLGARQEWQAIRRLHEAGVATMTAVAYGERGSDPARQHSFIVTEELAPTVDLEVFSQDWRERPPPPRLKRALVEAVARMVGDMHRAGVNHRDCYICHFLLHTDKPVSADDFRLSVIDLHRAQTRDATPKRWRNKDLAALYFSALDIGLTRRDKLRFLRTYFRRPLREILRDEAGLLAWMERKAEKLYERKQRYGDLL

SEQ ID NO: 57 - DNA, 795 bp, Salmonella typhimurium
atggttgagctgaaagcgccgttaaccacactatggcgcggtaaagatgcttttgaggaagtgaaaacgttacagggcgaagtgttcagagaactggagatgcgtcgaacattgcggtttgagctggacggcaaaagctacttcctgaagtggcataaaggcacttcgctgaaagaaattgtgaagaacctgatttcgttacgtatgcctgttctgggcgctgacagagaatggcacgccattcatcgtctacatgagctgggcgttgatacgatgcacggcgttggttttggtgaaaaaggcgtaaacccgctaaccagaacatcatttattatcaccgaagatttaacgcctaccattagccttgaagactactgtgttgactgggctgttaatccaccggacgcgcaggtgaagtggatgattattaagcgtgttgcgactatggtacgtaaaatgcacgccgggggaattaaccatcgcgactgttatatttgccactttcttctgcatttacctttcactggtcgcgaagaggatttaaaaatctccgtaatcgacctgcatcgcgcgcagatacgtcagcacgttccccttcgctggcgtgacaaagatttaattgggctttatttttcttcaatgaatattggcctgactcagcgagatatattccggtttatgcgtgagtatttctcactccccctgcgagagattttgcaaaaagactcggggttgattcatcaggcggatgttaaagccgctcgaattaaagaaagaacaataagaaaaaatctt

SEQ ID NO: 58 - protein, 265 aa, Salmonella typhimurium
MVELKAPLTTLWRGKDAFEEVKTLQGEVFRELEMRRTLRFELDGKSYFLKWHKGTSLKEIVKNLISLRMPVLGADREWHAIHRLHELGVDTMHGVGFGEKGVNPLTRTSFIITEDLTPTISLEDYCVDWAVNPPDAQVKWMIIKRVATMVRKMHAGGINHRDCYICHFLLHLPFTGREEDLKISVIDLHRAQIRQHVPLRWRDKDLIGLYFSSMNIGLTQRDIFRFMREYFSLPLREILQKDSGLIHQADVKAARIKERTIRKNL

SEQ ID NO: 59 - DNA, 30 bp, synthetic sequence
attcggatcctaagatgcctggcctggatg

SEQ ID NO: 60 - DNA, 30 bp, synthetic sequence
tcacgaattcacgacgagtctccagttcac

SEQ ID NO: 61 - DNA, 29 bp, synthetic sequence
atccgaattcaacatggcaagcgttaagg

SEQ ID NO: 62 - DNA, 30 bp, synthetic sequence
atccgtcgaccgaagagtccagccagattg

SEQ ID NO: 63 - DNA, 18 bp, synthetic sequence
aatacgctcggccttaac

SEQ ID NO: 64 - DNA, 1149 bp, Haemophilus influenzae
atgaaaataattttagtggtatttgtcttaatttttgtcggtgtcatcggttttaatatgataaaaggcgtaatgataagccgagccattgcaggaatgccagaatcttcaagcccagtaaccgcacttgaagttcaaccgcgtgaatggacgccagttattaacacaacaggtcttgtgcgtccaaatcaaggcgcaatgctcagtacacaaaatgcgggcgcggtttcacaagtacttgttcaaaatggacaaaatgtgaaaaaaggtgaggtgcttgtggagcttgatagttctgttgaacgagctaatctacaagctgctcaggcacaattatcagcacttcgtcaaacttaccaacgttatgtgggtttattaaatagcaatgctgtatcacgtcaagaaatggataacgcaaaagcggcttatgatgctcaagtagctagtattgaatctctaaaagcagcaattgaacgtcgtaaaattgttgcgccatttgatggcaaagcaggtattgtgaaaatcaatgttggacaatatgtgaatgttggaacagaaattgtgcgtgtagaagatactagctcaatgaaagtggattttgctctttcacaaaatgatttagataaattacatatcggtcagcgcgttacagcgacaacagatgctcgcttgggcgaaacattttcagctcgaatcactgcgattgaacctgccattaattcatcaacaggtttagttgatgttcaggctacttttgatcctgaagatgggcataaattgctttcaggtatgttctctcgcttacgcattgcacttccaactgaaacaaatcaagttgtcgttccacaagtagctattagctacaatatgtatggcgaaattgcctatttacttgaaccattatctgaagaagaaaaaggaaaaatgtcaggtaatgaaaaattggatcgtctctatcgtgcgaaacagatcaccgtatttactaaagatcgtcaaggtgtttatgctcaattacagggaaatgaagttaaagtgggagataaaattattacaggcggtcagcaaggtattggtaatggaagtcttgtggaatggattaaaaaagacattgtgggtgcaatagagcctgcacataaaacaccactttaa

SEQ ID NO: 65 - protein, 382 aa, Haemophilus influenzae
MKIILVVFVLIFVGVIGFNMIKGVMISRAIAGMPESSSPVTALEVQPREWTPVINTTGLVRPNQGAMLSTQNAGAVSQVLVQNGQNVKKGEVLVELDSSVERANLQAAQAQLSALRQTYQRYVGLLNSNAVSRQEMDNAKAAYDAQVASIESLKAAIERRKIVAPFDGKAGIVKINVGQYVNVGTEIVRVEDTSSMKVDFALSQNDLDKLHIGQRVTATTDARLGETFSARITAIEPAINSSTGLVDVQATFDPEDGHKLLSGMFSRLRIALPTETNQVVVPQVAISYNMYGEIAYLLEPLSEEEKGKMSGNEKLDRLYRAKQITVFTKDRQGVYAQLQGNEVKVGDKIITGGQQGIGNGSLVEWIKKDIVGAIEPAHKTPL

SEQ ID NO: 66 - DNA, 3099 bp, Haemophilus influenzae
atgtatgaggaaatccgaatgaaatttaccgatatatttattcgtcgtcctgttttagcagtttcaattagtttgttaatgattattttagggttgcaagcaatctcgaaattggcagtgcgtgaataccctaaaatgactactacagtcattacagtgagtaccgcatatccaggggcagatgcgaatttaatccaagcatttgttacgtcaaaattggaagaatctatcgcgcaagccgataatattgattatatgtcttcgactagtgcgcctagtagttcgactattacaataaaaatgaaattaaataccgatcctgcaggcgcgttagcagatgtgttagccaaagtgaatgcagtaaagtcagcattaccaaatggtattgaagatcctagtgtatcctcttcttcaggtgggagcggtattatgtatatcagtttccgatctaaaaaactagattctagtcaggtaactgattacatcaatcgtgtagtcaaaccacagttttttaccattgagggtgttgccgaagtacaggtatttggtgcagctgaatatgcattacgtatttggctagatccacaaaaaatggcagctcaaaatctttctgtgccaacagtgatgtctgccctttctgcaaataatgtacaaacggctgcggggaacgataatggctattatgtgagttatcgtaataaagtagagactacaacgaaatcagtggaacaactcagcaacttaattatttcatcaaatggcgatgatttagtgcgtttgcgtgatattgcaaccgttgaattaaataaagaaaatgataattcgcgtgctacggcaaatggtgcagagtccgttgtgttagccatcaatccaacctcgacagcaaaccctttgactgtcgcagaaaaaattcgccctttatatgaaagtataaaaacacaactgccagacagtatggaaagcgatattctttatgaccgcactattgccattaatagctctattcacgaggtcataaaaactattggtgaagctactttgatcgttttagtggtaattttaatgtttatcggttcgttccgtgctattttaattccaatattggctattccaatttctcttattggcgtattaatgctgctacaaagtttcaacttttctattaatttaatgactttgcttgctttaattcttgccataggtttagttgtagatgatgctattgtggtgctggagaatattgatcgtcatattaaagcgggagaaacgccattccgagcagcaattattggtacgcgtgaaattgcagtccctgttatttctatgactatcgcattgatcgcagtttactcaccaatggctttaatggggggcattactggcacattgtttaaagagtttgctttaacccttgctggtgcagtatttatttctggtgttgtggcattaacgttatcgccaatgatgagtagtaagttactcaaatccaatgctaaaccaacatggatggaagaacgcgtagaacataccttaggtaaagtaaatcgtgtttacgaatacatgcttgatctcgttatgctcaatcgtaaatcaatgctggcttttgcggtcgtgattttctcaacgctcccatttttgtttaattcactttctagtgaattaacgccaaatgaagataaaggcgcatttattgcaattggtaacgcgccatctagcgtaaatgtggattacattcaaaatgcaatgcaaccatatatgaaaaatgtaatggaaacacccgaagtttcttttggtatgagcattgctggtgcgccaacttctaatagctcgttaaatatcatcacattgaaagattggaaagaacgttcacgtaaacaatccgcaataatgaatgaaattaatgaaaaagcaaaatcaattccagaagtgtcagtatcagcatttaacattcctgaaattgatacaggggaacaaggccctccagtctctattgtattgaaaactgcacaagattataaatctttggcaaacaccgcagagaaattcctcagtgcgatgaaagcctctggcaaatttatttatacaaatttagatttgacctatgacactgcacaaatgactatttctgtggataaagaaaaagcgggaacttacggtattacaatgcaacaaatcagtaatactttagggagtttcttatctggtgcgacagttacgcgtgtggatgtggatggacgcgcttataaagtcatttcgcaggtaaaacgagatgatcgcttatcgccagaaagtttccaaaattattatttaactgcatctaatggtcaatcagtgccattaagtagtgttattagtatgaaattagaaactcagccaacatcattaccgcgtttcagtcagttaaattcggctgaaatcagtgctgtaccaatgccgggtatatcaagtggtgatgccattgcttggcttcaacaacaggcaacagacaatttaccgcaaggctatacgtttgattttaaatctgaagcacgtcaattagtccaagaaggcaacgcattagccgtcacttttgcattggctgttatcatcatattcttggtacttgccattcagtttgaatctatacgtgacccaatggtaattatgatttctgtaccattagccgtaagtggtgcattggtgagcttaaatattttatccttctttagcatcgcaggaacaacattaaatatctactctcaagttgggttgattactctcgtgggattaatcaccaaacacggtatcttaatgtgcgaagtggcaaaagaagaacagcttaaccatggtaaaactcgaattgaggcaatcactcatgctgcaaaagtacgtttacgcccaatcctaatgacaacggcggcaatggtagctggcttaattccattactctatgcaacgggtgcaggagcggtatctcgctttagtattgggatagtgattgtagcgggattatccattggtactattttcaccttgttcgtcttgcctgtagtatatagctatgtcgcaactgaacacaaaccattaccggtttttgatgaaaataaaacaacccactaa

SEQ ID NO: 67 - protein, 1032 aa, Haemophilus influenzae
MYEEIRMKFTDIFIRRPVLAVSISLLMIILGLQAISKLAVREYPKMTTTVITVSTAYPGADANLIQAFVTSKLEESIAQADNIDYMSSTSAPSSSTITIKMKLNTDPAGALADVLAKVNAVKSALPNGIEDPSVSSSSGGSGIMYISFRSKKLDSSQVTDYINRVVKPQFFTIEGVAEVQVFGAAEYALRIWLDPQKMAAQNLSVPTVMSALSANNVQTAAGNDNGYYVSYRNKVETTTKSVEQLSNLIISSNGDDLVRLRDIATVELNKENDNSRATANGAESVVLAINPTSTANPLTVAEKIRPLYESIKTQLPDSMESDILYDRTIAINSSIHEVIKTIGEATLIVLVVILMFIGSFRAILIPILAIPISLIGVLMLLQSFNFSINLMTLLALILAIGLVVDDAIVVLENIDRHIKAGETPFRAAIIGTREIAVPVISMTIALIAVYSPMALMGGITGTLFKEFALTLAGAVFISGVVALTLSPMMSSKLLKSNAKPTWMEERVEHTLGKVNRVYEYMLDLVMLNRKSMLAFAVVIFSTLPFLFNSLSSELTPNEDKGAFIAIGNAPSSVNVDYIQNAMQPYMKNVMETPEVSFGMSIAGAPTSNSSLNIITLKDWKERSRKQSAIMNEINEKAKSIPEVSVSAFNIPEIDTGEQGPPVSIVLKTAQDYKSLANTAEKFLSAMKASGKFIYTNLDLTYDTAQMTISVDKEKAGTYGITMQQISNTLGSFLSGATVTRVDVDGRAYKVISQVKRDDRLSPESFQNYYLTASNGQSVPLSSVISMKLETQPTSLPRFSQLNSAEISAVPMPGISSGDAIAWLQQQATDNLPQGYTFDFKSEARQLVQEGNALAVTFALAVIIIFLVLAIQFESIRDPMVIMISVPLAVSGALVSLNILSFFSIAGTTLNIYSQVGLITLVGLITKHGILMCEVAKEEQLNHGKTRIEAITHAAKVRLRPILMTTAAMVAGLIPLLYATGAGAVSRFSIGIVIVAGLSIGTIFTLFVLPVVYSYVATEHKPLPVFDENKTTH